SEQUENCE LISTING (50 sequences)

SEQ ID NO: 1 - DNA, 783 bp, Zea mays
atggggcggtcgccgtgctgcgagaaggcgcacaccaacaggggcgcgtggaccaaggaggaggacgagcggctggtggcctacgtccgcgcgcacggcgaagggtgctggcgctcgctgcccagggcggcgggcctgctgcgctgcggcaagagctgccgcctgcgctggatcaactacctccgcccggacctcaagcgaggcaacttcaccgccgacgaggacgacctcatcgtcaagctgcacagcctcctcgggaacaagtggtcgctcatcgccgcgcggctcccggggcggacggacaacgagatcaagaactactggaacacgcacatccggcgcaagctgctgggcagcggcatcgaccccgtcacgcaccgccgcgtcgcggggggcgccgcgaccaccatctcgttccagcccagccccaacaccgccgtcgccgccgccgcagaaacagcagcgcaggcgccgatcaaggccgaggagacggcggccgtcaaggcgcccaggtgccccgacctcaacctggacctctgcatcagcccgccgtgccagcatgaggacgacggcgaggaggaggaggaggagctggacctcatcaagcccgccgtcgtcaagcgggaggcgctgcaggccggccacggccacggccacggcctctgcctcggctgcggcctgggcggacagaagggagcggccgggtgcagctgcagcaacggccaccacttcctggggctcaggaccagcgtgctcgacttcagaggcctggagatgaagtga

SEQ ID NO: 2 - protein, 260 aa, Zea mays
MGRSPCCEKAHTNRGAWTKEEDERLVAYVRAHGEGCWRSLPRAAGLLRCGKSCRLRWINYLRPDLKRGNFTADEDDLIVKLHSLLGNKWSLIAARLPGRTDNEIKNYWNTHIRRKLLGSGIDPVTHRRVAGGAATTISFQPSPNTAVAAAAETAAQAPIKAEETAAVKAPRCPDLNLDLCISPPCQHEDDGEEEEEELDLIKPAVVKREALQAGHGHGHGLCLGCGLGGQKGAAGCSCSNGHHFLGLRTSVLDFRGLEMK

SEQ ID NO: 3 - DNA, 1129 bp, Zea mays
cgggcgcaggaattcggcacgaggggaaacccgcgcccactcgctgccttctcaaatccaaacgcgaagtagcaacaagcaaaagcccagatcgataatacgatggggcggtcgccgtgctgcgagaaggcgcacaccaacaggggcgcgtggaccaaggaggaggacgagcggctggtggcctacgtccgcgcgcacggcgaagggtgctggcgctcgctgcccagggcggcgggcctgctgcgctgcggcaagagctgccgcctgcgctggatcaactacctccgcccggacctcaagcgaggcaacttcaccgccgacgaggacgacctcatcgtcaagctgcacagcctcctcgggaacaagtggtcgctcatcgccgcgcggctcccggggcggacggacaacgagatcaagaactactggaacacgcacatccggcgcaagctgctgggcagcggcatcgaccccgtcacgcaccgccgcgtcgcggggggcgccgcgaccaccatctcgttccagcccagccccaactccgccgccgccgccgccgccgcagaaacagcagcgcaggcgccgatcaaggccgaggagacggcggccgtcaaggcgcccaggtgccccgacctcaacctggacctctgcatcagcccgccgtgccagcatgaggacgacggcgaggaggaggacgaggagctggacctcaagcccgccttcgtcaagcgggaggcgctgcaggccggccacggccacggccacggcctctgcctcggctgcggcctgggcggacagaagggagcggccgggtgcagctgcagcaacggccaccacttcctggggctcaggaccagcgtgctcgacttcagaggcctggagatgaagtgaacgaaacgaagcccacacgtcctttcttctccttttgttgtcggttgtagtcttggcttgttggatttggatagagctagttggttactagttgttagttagaagatagtgcaggatgatcactagctactggctacctcaacagtacagtagctgctcccttctcttccattctatgtaaaaaagaaacaaaaatacttataaggtgtttgggttgagaaatgaactagtctattatcttttc

SEQ ID NO: 4 - protein, 260 aa, Zea mays
MGRSPCCEKAHTNRGAWTKEEDERLVAYVRAHGEGCWRSLPRAAGLLRCGKSCRLRWINYLRPDLKRGNFTADEDDLIVKLHSLLGNKWSLIAARLPGRTDNEIKNYWNTHIRRKLLGSGIDPVTHRRVAGGAATTISFQPSPNSAAAAAAAETAAQAPIKAEETAAVKAPRCPDLNLDLCISPPCQHEDDGEEEDEELDLKPAFVKREALQAGHGHGHGLCLGCGLGGQKGAAGCSCSNGHHFLGLRTSVLDFRGLEMK

SEQ ID NO: 5 - DNA, 783 bp, Oryza sativa
atggccgaggcggcgtcggcggcggcggcggcgacgacggagcaggcgaatgggagcagcggcggcgagcagaagacgcggcactcggaggtcggccacaagagcctcctcaagagcgacgatctctaccagtacatcctggagacgagcgtgtacccgcgcgagcacgagtgcatgaaggagctccgcgaggtcaccgccaaccacccatggaacctgatgacgacgtcggcggacgaggggcaattcctgaacctgctgctgaagctcatcggcgccaagaagaccatggagatcggcgtctacaccggctactccctcctcgccaccgccctcgccatccccgacgacggcacgatcttggcgatggacatcaaccgggagaactacgagctggggctcccgtcgatcgagaaggcgggagtggcgcacaagatcgacttccgggagggacccgcgctgccggtgctggaccagctggtggaggaggagggcaaccatgggtcgttcgacttcgtgttcgtcgacgccgacaaggacaactacctcaactaccacgagcggctgatgaagctggtcaaggtcggcggcctcgtcggctacgacaacacgctctggaacggctccgtcgtgctccccgccgacgcccccatgcgcaagtacatccgctactaccgcgacttcgtgctcgagctcaacaaggccctcgccgccgaccaccgcgtcgagatctgccagctccccgtcggcgacggcatcaccctctgccgccgcgtcaagtga

SEQ ID NO: 6 - protein, 260 aa, Oryza sativa
MAEAASAAAAATTEQANGSSGGEQKTRHSEVGHKSLLKSDDLYQYILETSVYPREHECMKELREVTANHPWNLMTTSADEGQFLNLLLKLIGAKKTMEIGVYTGYSLLATALAIPDDGTILAMDINRENYELGLPSIEKAGVAHKIDFREGPALPVLDQLVEEEGNHGSFDFVFVDADKDNYLNYHERLMKLVKVGGLVGYDNTLWNGSVVLPADAPMRKYIRYYRDFVLELNKALAADHRVEICQLPVGDGITLCRRVK

SEQ ID NO: 7 - DNA, 1034 bp, Oryza sativa
  promoter (1)..(1034): transcription regulating sequence of caffeoyl CoA-O-methyltransferase gene including 5′-untranslated region
caactactgcacggtaaaagtgataggaatcggtcggaaacagtattaatgtttttattatttttacaaaaacgaattgaaataattggaaattttcatatttatatattaaactattcagtatcaacttcaattcgacgtcaatagaaattagaaaagcataattatacacagtaataggcgttcaagatattattgttattatttagttttgtggaaatggtatcaacgtgatcggaaaattttgtacatgttttcaccctgcgggatatctcaattccttctcctccctctaccgccatatcagcacacgttttagagcaccaatcataacccataaatccgtgggctactcacttatttaatttatatgtgaattcgtgacctgactcactcacatactatcaaaaatttgtctcagtcacccatctccttctttcctggtccgataagggtttatcctacggttcgacggttatcacgatagtcgtgcggttactgaggtataccgtgatttaaaaatatgataaagttaccgcaggttttaactgcgcggtttggtaaacctgttcctcctcaccaaccttctcctccggtctccttatgtgtctcaccgaggcgagccgccgcgagaccgcatggacgcggtccacgcacctggcggtgcacctcctcctccccggcgaagaagacgtggaggagagtaaatgagcaatcaggcccacggcccaatcgccgtccaccacccaccaccctcagcgacccaaaaccacctcaccaacccaactctgtaccgtactgtacccgccctcccctcccactgacactccgggcccacctgtcggcgcgactcttccacggtccccttctctcctcagagattttttccacgcattttttaatttttttttctgcagttcacatgctcttctcccactcttccgccgcgctatataaaccgcgcgaggcgtcgttgcctcgtcggcgaagtcaatccggcgatccccggcgagcgagagatcgaagcaagctgc

SEQ ID NO: 8 - DNA, 992 bp, Oryza sativa
  promoter (1)..(992): transcription regulating sequence from caffeoyl CoA-O-methyltransferase gene
gcaactactgcacggtaaaagtgataggaatcggtcggaaacagtattaatgtttttattatttttacaaaaacgaattgaaataattggaaattttcatatttatatattaaactattcagtatcaacttcaattcgacgtcaatagaaattagaaaagcataattatacacagtaataggcgttcaagatattattgttattatttagttttgtggaaatggtatcaacgtgatcggaaaattttgtacatgttttcaccctgcgggatatctcaattccttctcctccctctaccgccatatcagcacacgttttagagcaccaatcataacccataaatccgtgggctactcacttatttaatttatatgtgaattcgtgacctgactcactcacatactatcaaaaatttgtctcagtcacccatctccttctttcctggtccgataagggtttatcctacggttcgacggttatcacgatagtcgtgcggttactgaggtataccgtgatttaaaaatatgataaagttaccgcaggttttaactgcgcggtttggtaaacctgttcctcctcaccaaccttctcctccggtctccttatgtgtctcaccgaggcgagccgccgcgagaccgcatggacgcggtccacgcacctggcggtgcacctcctcctccccggcgaagaagacgtggaggagagtaaatgagcaatcaggcccacggcccaatcgccgtccaccacccaccaccctcagcgacccaaaaccacctcaccaacccaactctgtaccgtactgtacccgccctcccctcccactgacactccgggcccacctgtcggcgcgactcttccacggtccccttctctcctcagagattttttccacgcattttttaatttttttttctgcagttcacatgctcttctcccactcttccgccgcgctatataaaccgcgcgaggcgtcgttgcctcgtcggcgaagt

SEQ ID NO: 9 - DNA, 301 bp, Oryza sativa
  promoter (1)..(301): Potential core region of the transcription regulating sequence of caffeoyl CoA-O-methyltransferase gene comprising clusters of promoter elements
acgtggaggagagtaaatgagcaatcaggcccacggcccaatcgccgtccaccacccaccaccctcagcgacccaaaaccacctcaccaacccaactctgtaccgtactgtacccgccctcccctcccactgacactccgggcccacctgtcggcgcgactcttccacggtccccttctctcctcagagattttttccacgcattttttaatttttttttctgcagttcacatgctcttctcccactcttccgccgcgctatataaaccgcgcgaggcgtcgttgcctcgtcggcgaagtc

SEQ ID NO: 10 - DNA, 1093 bp, Oryza sativa
gccgatgcccaagaactagtcattttaaatttataaattataactaaattgtataatttttgccttttttttttaatttgcaagctactggaaaatgttatttaatatatgtataaatgtcgagacaataatattattgcattataaattgacctggtttttgttagctttcgttgcgcgtgagaatggtgagtgtgtgctgctgatgaaactcgaatgttcacttttgttgtcttgtccagctttgctaaactttggcagcattagcaaagctgttttgttctgtttctgaattgttcttggattgaaatctctaatattgacttgataattaaatttgaccggtttttggcagtaaattccttcagtattggcagctcaaactgaattcttctactaatagtttagtgcttgtggagagtgtagtcgtgtagagtggactggactaattcagatctttacttggttagctgaagatggcatccgttatctgaattcttcagtagttggtggataatgacaactcgatgtagagagataatttgctgcatgttagtttggaagtagcttcaaaggatttaattctcagcgtccgaagtcttaagtacagttggtttggagagctgttcctgtgaagacttttatgatttgtgctagttatgtcatcacatgatcacttcaattatcacttatcgatctagtgagaccaaccatacataccatacaaaggtaaaaagtgttcaaactggattgaacaagttctgtctccatatagatcctactaaaatgcatacatgttgtagcaatcccatttcatccaaaccaaacaaaatctctttcattcggctctaaccaatcaaacagagccatttgtatcccccgaaccaaaccagccgagcatggatgagggatcgagggcatccgagcaaccaaacaggcccaaagtgaatgctttggtcgattttcgatttgttccctacgaatccaattttagtccttcagccagaagaccagatacaattgatccctcaactatcaaaacaagtgcagatgagctccctcgacagtttggacggcggtttggttgacgtgg

SEQ ID NO: 11 - DNA, 1554 bp, Zea mays
cttcgtcgcatcgcacgccatcagcagctagcgcgctcctcgagcccagcagagaaaggccggcctacccactctctctctctctctctccagtctccaccggcagcgctaatcgtaatagccatgggctccaccgccggcgacgtggccgcggtggtggacgaggaggcgtgcatgtacgcgatgcagctggcgtcgtcgtccatcctgcccatgacgctgaagaacgccatcgagctgggcctgctggaggtgctgcagaaggaggccggcggcggcaaggcggcgctggcgcccgaggaggtggtggcgcggatgcccgcggcgcccagcgaccccgccgccgccgcggccatggtggaccgcatgctccgcctgctcgcctcctacgacgtcgtccggtgccagatggaggaccgggacggccggtacgagcgccgctactccgccgcgcccgtctgcaagtggctcacccccaacgaggacggcgtgtccatggccgccctcgcgctcatgaaccaggacaaggtcctcatggagagctggtactacctcaaggacgcggtgctggacggcggcatcccgttcaacaaggcgtacgggatgacggcgttcgagtaccacggcacggacgcgcgcttcaaccgcgtgttcaacgagggcatgaagaaccactcggtgatcatcaccaagaagctgctggacttctacacgggcttcgagggcgtgtcgacgctggtggacgtgggcggcggcgtgggcgccacgctgcacgccatcacgtcccgccacccgcacatctccggggtcaacttcgacctgccgcacgtcatctccgaggcgccgccgttccccggcgtgcgccacgtgggcggggacatgttcgcgtccgtgcccgccggcgacgccatcctcatgaagtggatcctccacgactggagcgacgcgcactgcgccacgctgctcaagaactgctacgacgcgctgccggaaaatggcaaggtcatcgtcgtcgagtgcgtgctgccggtcaacacggaggccacccccaaggcgcagggcgtgttccacgtcgacatgatcatgctcgcgcacaaccccggcggcaaggagcggtacgagcgcgagttccgcgagctcgccaagggcgccggcttctccgggttcaaggccacctacatctacgccaacgcctgggccatcgagttcatcaagtgaatacggctaccaccgtcgccgcgatgagatgcatggctgccacatgcatgcttgcttgcttggtcctcgtatcgtacgtcgccgtcgtcgtcttcttctggttgcgctgctaccttgctgctctcgccctcgcgtatgcatgtacttttgcttaattttctttcttcatatcatgcactctggctggcctagactgcccccgatctatgctggccggtacgtcttgtcgagctcttgcatgtcgtggattctaaattcttcttctgcgtcgaattgtctctgccatgtgcgagtaataacaatcaaggttatacttaccatacaattacatggtgg

SEQ ID NO: 12 - protein, 364 aa, Zea mays
MGSTAGDVAAVVDEEACMYAMQLASSSILPMTLKNAIELGLLEVLQKEAGGGKAALAPEEVVARMPAAPSDPAAAAAMVDRMLRLLASYDVVRCQMEDRDGRYERRYSAAPVCKWLTPNEDGVSMAALALMNQDKVLMESWYYLKDAVLDGGIPFNKAYGMTAFEYHGTDARFNRVFNEGMKNHSVIITKKLLDFYTGFEGVSTLVDVGGGVGATLHAITSRHPHISGVNFDLPHVISEAPPFPGVRHVGGDMFASVPAGDAILMKWILHDWSDAHCATLLKNCYDALPENGKVIVVECVLPVNTEATPKAQGVFHVDMIMLAHNPGGKERYEREFRELAKGAGFSGFKATYIYANAWAIEFIK

SEQ ID NO: 13 - DNA, 1843 bp, Zea mays
taaggagatgatctcaggctatctcctatatcatccccttcatttttcaaacaatgtcatatacagtgtaaacacgctttactgaagacaactttaaggtagttatagtgtgaaggagtgtgagctgatctaagaaaactattctttccgttcaactttttagcttgattattcaaaatgatatataaataagaacatttagctataatataaacttttacatattttaatggcaaagaatagatttcgacacataataaacacattagtaaactaataattactcaaaccatgatttttgatatggtgaaacataaggttgccaagatcgcaatttaaatcctaaatcattatcatatacaaatatatggttataccactagtagaaacacaactctatgtctctggtggacactaatttttacggacggtttcagttatcgagcacatgtgaaagtaaaatggttagctcgactcaagaaccgttagtggaaacacctttacactaacagtttccttaaaaccctgacagtgcaaatcgagcatttccactatcgactttcttaagaaatcatatttgtactagtgggtccttaagaagaccgctagatttgtactgccggttttttaaggacctgacaatacatatcatttgaatttttttaaagtttttcaaataaactcatatggaaaaaccaccgaaacaaaaaagttgtagatctctaaaagttatgaaacttttaattgacaattttttttgaaattattagggctcccaaaattgtatctgaatttttcaaatttaaatgtaaattttgcaaacgacctcggaatgataaaataccaaaataaaagttatagatcctgaaaaagtcattcaactttatagttaacaactttttcgtttaaatttgtttagtgcccccaaacaatcgatttacacttggtttgttctaatatgtggataacaaacctataatctaaacataaatcagtgatggatagagtggtagagaaggctacacacgagagtgaggtcggagtttcgattcaaactagcatggtttatattttcaaaaagatgtgtattgacggttttcttaacgtagtcgccaataaaatttgatttccacggcacctgccaccggcccaccgtcgtcatcgctgaccaacccggctggtcgcctctctgttccatccatgcatgttacaactatgcacatgcagccgaaacaaacactggctagaaaggcagcccaacgggcctactgtcattcgctccggcatcctactggtgggcccacttgcaccggccgatgaccagttcatcatttttctcgacgattttgtgcacagaatttgctaaaaattcttcgcacgtggcaaaaccaggggaaaaatcgacaactagtcgggtttttttgctgatagaatagtccctgctattcatccatgaaaaccaaacacgtacactacgtcaccgtcatggatgggagcgagtgaactgatgatttttttccccaccccgcacgcaacagcatgggtgacaacaaccactcccgctgcggttgggcgagcacatctctacgcacttgacactcacgcaaacctaacgcatactagattaatcatccatcgccaccaactatcggcaacagaaacgatgggccccgcttctcttaatcacggtgcttgaattagtgcgcgcatagtagtgaaaaaaagcagtgcgtgttttggtgtggtggttggtgagccgtccggcccaataaaaacccctcgcaccacctcgtccctcttcgtcgcatcgcacgccatcagcagctagcgcgctcctcgatgg

SEQ ID NO: 14 - DNA, 2748 bp, Zea mays
tccacggcagctgccaccgtcgctatcgctgaccaacccggctggtcgcctctgtgctccatccatgcatgttacaactatgcagatgcagccgaaacaaacactggctagaaaggcagcccaacgggcctactgtcattcgctccggcatcctactggtgggcccacttgcaccggccgatgaccagttcatcatttttctcgacgaatttgtgcacagaatttgctaaaaattcttcgcacgtggcaaaaccagggggaaaatcgacaactagtcggggtttttttaattccctgatagaatagtccctgctaatcatccatgaaaaccaaacacgtactctacgtcaccgtcatggatggagcgagtgaactgatgattttttccccatcccgcacgcaacagcatgggtgacaacaaccactcccgctgcggttgggcgagcacatctctacgcacttgacactcacgcaaacctaacgcatactagagtaatcatcgccaccaactatcggcgacagaaacgatgggccccgcttctcttaatcacggtgcttgaattagtgcgcgcatagtagtgaaaaataatagtgaaaaataagcagtgcgtgttttggtgtggtggttggtgagccgtccggcccaataaaaacccctcgcaccacctcgtccctcttcgtcgcatcgcacgccatcagcagctagcgcgctcctcgagcccagcagagaaaggccggcagcgctaatcgtaatagccatgggctccaccgccggcgacgtggccgcggtggtggacgaggaggcgtgcatgtacgcgatgcagctggcgtcgtcgtccatcctgcccatgacgctgaagaacgccatcgagctgggcctgctggaggtgctgcagaaggaggccggcggcggcaaggcggcgctggcgcccgaggaggtggtggcgcggatgcccgcggcgcccagcgaccccgccgccgccgcggccatggtggaccgcatgctccgcctgctcgcctcctacgacgtcgtccggtgccagatggaggaccgggacggccggtacgagcgccgctactccgccgcgcccgtctgcaagtggctcacccccaacgaggacggcgtgtccatggccgccctcgcgctcatgaaccaggacaaggtcctcatggagagctggtgagtagtagccgcatcgcatcaaccaccttctacctatctatatccatcacttgttgctgctggcgtgcgcggcatgcatgatgacgagctcgctcatcattggtgctactagtgatttatttcgtccagtaaaattaattaaggtgcgctgctactctactggctgcggctagcacaaggctggaaatagttgttacttgttatacacgatataatatttctctagaacaaaaaagattttttttttataaaaagcaagcaagaaagaaagtgagtgacttcatgtttttcctaaaaaaaagttaggagtgggatggaaaagtcagcaaggaccacttgtttgttgtccactatccatccagtgggtgagacttttttgcgagacggagcactatattattggccgagtcccttttctgtatccgcaaaacggcagccgtcgatcgccggacggatcgcacggcgacactgagtgtcgagtccaattccaaccacgagggctggaaggaaagccatccgtgctggtctggactttttgccaaactccattcagacgttcgccgactgaaggtgaatcttcagacacagccagattgtttggtgtctgcgaagatggcgtagaaaagaccaagagacagttggctcacacagacaagtgacaactgactatagtatctgcctgcctggctgatgctgatagagatagactcttgccctgtctgttccttgtacaaacgtggacaaacattagtagcagttttctttgatctacaaaaagtactacctcagtttttaaatatttatcggctgttagtttatttttgaacttaaacgactggttgcaaaagatagatagatacaaacgaaaggatgtcgtcgctgtgcgctgatctgatcactgccactctgccaggtactatctcaaggacgcggtgctggacggcggcatcccgttcaacaaggcgtacgggatgacggcgttcgagtaccacggcacggacgcgcgcttcaaccgcgtgttcaacgagggcatgaagaaccactcggtgatcatcaccaagaagctgctggacttctacacgggcttcgagggcgtgtcgacgctggtggacgtgggcggcggcgtgggcgccacgctgcacgccatcacgtcccgccacccgcacatctccggggtcaacttcgacctgccgcacgtcatctccgaggcgccgccgttccccggcgtgcgccacgtgggcggggacatgttcgcgtccgtgcccgccggcgacgccatcctcatgaagtggatcctccacgactggagcgacgcgcactgcgccacgctgctcaagaactgctacgacgcgctgccggaaaatggcaaggtcatcgtcgtcgagtgcgtgctgccggtcaacacggaggccacccccaaggcgcagggcgtgttccacgtcgacatgatcatgctcgcgcacaaccccggcggcaaggagcggtacgagcgcgagttccgcgagctcgccaagggcgccggcttctccgggttcaaggccacctacatctacgccaacgcctgg

SEQ ID NO: 15 - DNA, 2512 bp, Zea mays
ggtgagccgtccggcccaataaaaacccctcgcaccacctcgtccctcttcgtcgcatcgcacgccatcagcagctagcgcgctcctcgagcccagcagagaaaggccggcagcgctaatcgtaatagccatgggctccaccgccggcgacgtggccgcggtggtggacgaggaggcgtgcatgtacgcgatgcagctggcgtcgtcgtccatcctgcccatgacgctgaagaacgccatcgagctgggcctgctggaggtgctgcagaaggaggccggcggcggcaaggcggcgctggcgcccgaggaggtggtggcgcggatgcccgcggcgcccagcgaccccgccgccgccgcggccatggtggaccgcatgctccgcctgctcgcctcctacgacgtcgtccggtgccagatggaggaccgggacggccggtacgagcgccgctactccgccgcgcccgtctgcaagtggctcacccccaacgaggacggcgtgtccatggccgccctcgcgctcatgaaccaggacaaggtcctcatggagagctggtgagtagtagccgcatcgcatcaaccaccttctacctatctatatccatcacttgttgctgctggcgtgcgcggcatgcatgatgacgagctcgctcatcattggtgctactagtgatttatttcgtccagtaaaattaattaaggtgcgctgctactctactggctgcggctagcacaaggctggaaatagttgttacttgttatacacgatataatatttctctagaacaaaaaagattttttttttataaaaagcaagcaagaaagaaagtgagtgacttcatgtttttcctaaaaaaaagttaggagtgggatggaaaagtcagcaaggaccacttgtttgttgtccactatccatccagtgggtgagacttttttgcgagacggagcactatattattggccgagtcccttttctgtatccgcaaaacggcagccgtcgatcgccggacggatcgcacggcgacactgagtgtcgagtccaattccaaccacgagggctggaaggaaagccatccgtgctggtctggactttttgccaaactccattcagacgttcgccgactgaaggtgaatcttcagacacagccagattgtttggtgtctgcgaagatggcgtagaaaagaccaagagacagttggctcacacagacaagtgacaactgactatagtatctgcctgcctggctgatgctgatagagatagactcttgccctgtctgttccttgtacaaacgtggacaaacattagtagcagttttctttgatctacaaaaagtactacctcagtttttaaatatttatcggctgttagtttatttttgaacttaaacgactggttgcaaaagatagatagatacaaacgaaaggatgtcgtcgctgtgcgctgatctgatcactgccactctgccaggtactatctcaaggacgcggtgctggacggcggcatcccgttcaacaaggcgtacgggatgacggcgttcgagtaccacggcacggacgcgcgcttcaaccgcgtgttcaacgagggcatgaagaaccactcggtgatcatcaccaagaagctgctggacttctacacgggcttcgagggcgtgtcgacgctggtggacgtgggcggcggcgtgggcgccacgctgcacgccatcacgtcccgccacccgcacatctccggggtcaacttcgacctgccgcacgtcatctccgaggcgccgccgttccccggcgtgcgccacgtgggcggggacatgttcgcgtccgtgcccgccggcgacgccatcctcatgaagtggatcctccacgactggagcgacgcgcactgcgccacgctgctcaagaactgctacgacgcgctgccggaaaatggcaaggtcatcgtcgtcgagtgcgtgctgccggtcaacacggaggccacccccaaggcgcagggcgtgttccacgtcgacatgatcatgctcgcgcacaaccccggcggcaaggagcggtacgagcgcgagttccgcgagctcgccaagggcgccggcttctccgggttcaaggccacctacatctacgccaacgcctgggccatcgagttcatcaagtgaatacggctaccaccgtcgccgcgatgagatgcatggctgccacatgcatgcttgcttgcttggtcctcgtatcgtacgtcgccgtcgtcgtcttcttctggttgcgctgctaccttgctgctctcgccctcgcgtatgcatgtacttttgcttaattttctttcttcatatcatgcactctggctggcctagactgcccccgatccatggtggccatgtcttcggtacgtcttgtcgagctcttgcatgtcgtggattctaaattcttcttctgcgtcgaattgtctctgccatgtgcgagtaataacaatcaaggttatacttaccatacaattacatggtggtttaattgctctcttttaatttggtga

SEQ ID NO: 16 - DNA, 693 bp, Zea mays
gcagcgtcggtggttgcttcatggctgtcgaggggaatgacgtccggtccgaacaagccacggctgctgctgcgctaccgccgcggcttcggaccaggcttcattccccacgactcaccatggaaacggcagcgaggtgcgagcttccagtgcgcgcagtgtttgatcagcaaacacctgcgtggccgaggctgagccttgcaagggcaggcgtccatgttctcgtcagcaagcgtcttcttcacatgtctgaggtctgagcaattgcgggtggacatcgatggatgagcattgccgctgcaagggaagatcgtccggctcacacttgagttgggataccatgcactactcttgttgtagttagattagataccatgcgcttgaaccgtgcaatagcatagcgaggaagaggaacccagagaggactcagaaagagatgttgtctatttcatccagtgctcatctagtcatctagtctgcctgcgggtgtctgcaatgaacgctgacgctgctgccggagacctactctgctactggtgtggagtggacaaacagggagggaaggcgtctgctagaacctagaagacggacacagggaggaccaacaggactaaagaccgtcgtacgtgttgaagcggaaggactttcttattacgtgtggcgcttcgaattaaagccagcggttagaacggc

SEQ ID NO: 17 - DNA, 21 bp, Zea mays
ggggaatgacgtccggtccga

SEQ ID NO: 18 - DNA, 21 bp, Zea mays
tcggaccaggcttcattcccc

SEQ ID NO: 19 - DNA, 21 bp, Zea mays
tgcatgtcgtggattccaaca

SEQ ID NO: 20 - DNA, 21 bp, Artificial Sequence
  miRNA Bm3d
ttagaatccacgacatgcatc

SEQ ID NO: 21 - DNA, 21 bp, Artificial Sequence
  Star sequence of miRNA Bm3E
acgacgcgctgccggacaaca

SEQ ID NO: 22 - DNA, 21 bp, Artificial Sequence
  miRNA Bm3e
ttttccggcagcgcgtcgtac

SEQ ID NO: 23 - DNA, 374 bp, Artificial Sequence
  pr-Zm pre-miR166m (374) Bm3d
gcagcgtcggtggttgcttcatggctgtcgaggatgcatgtcgtggattccaacaagccacggctgctgctgcgctaccgccgcggcttagaatccacgacatgcatccacgactcaccatggaaacggcagcgaggtgcgagcttccagtgcgcgcagtgtttgatcagcaaacacctgcgtggccgaggctgagccttgcaagggcaggcgtccatgttctcgtcagcaagcgtcttcttcacatgtctgaggtctgagcaattgcgggtggacatcgatggatgagcattgccgctgcaagggaagatcgtccggctcacacttgagttgggataccatgcactactcttgttgtagttagattagatacc

SEQ ID NO: 24 - DNA, 175 bp, Artificial Sequence
  pr-Zm pre-miR166s (175) Bm3d
gcagcgtcggtggttgcttcatggctgtcgaggatgcatgtcgtggattccaacaagccacggctgctgctgcgctaccgccgcggcttagaatccacgacatgcatccacgactcaccatggaaacggcagcgaggtgcgagcttccagtgcgcgcagtgtttgatcagcaaac

SEQ ID NO: 25 - DNA, 175 bp, Artificial Sequence
  pr-Zm pre-miR166s (175 32C 109G) Bm3e
gcagcgtcggtggttgcttcatggctgtcgacgtacgacgcgctgccggacaacaagccacggctgctgctgcgctaccgccgcggcttttccggcagcgcgtcgtacgacgactcaccatggaaacggcagcgaggtgcgagcttccagtgcgcgcagtgtttgatcagcaaac

SEQ ID NO: 26 - DNA, 358 bp, Artificial Sequence
  pr-2xZm pre-miR Bm3d_Bm3e
gcagcgtcggtggttgcttcatggctgtcgaggatgcatgtcgtggattccaacaagccacggctgctgctgcgctaccgccgcggcttagaatccacgacatgcatccacgactcaccatggaaacggcagcgaggtgcgagcttccagtgcgcgcagtgtttgatcagcaaacatttaaatgcagcgtcggtggttgcttcatggctgtcgacgtacgacgcgctgccggacaacaagccacggctgctgctgcgctaccgccgcggcttttccggcagcgcgtcgtacgacgactcaccatggaaacggcagcgaggtgcgagcttccagtgcgcgcagtgtttgatcagcaaac

SEQ ID NO: 27 - DNA, 1421 bp, Sugarcane bacilliform virus
gaagttgaagacaaagaaggtcttaaatcctggctagcaacactgaactatgccagaaaccacatcaaagatatgggcaagcttcttggcccattatatccaaagacctcagagaaaggtgagcgaaggctcaattcagaagattggaagctgatcaataggatcaagacaatggtgagaacgcttccaaatctcactattccaccagaagatgcatacattatcattgaaacagatgcatgtgcaactggatggggagcagtatgcaagtggaagaaaaacaaggcagacccaagaaatacagagcaaatctgtaggtatgccagtggaaaatttgataagccaaaaggaacctgtgatgcagaaatctatggggttatgaatggcttagaaaagatgagattgttctacttggacaaaagagagatcacagtcagaactgacagtagtgcaatcgaaaggttctacaacaagagtgctgaacacaagccttctgagatcagatggatcaggttcatggactacatcactggtgcaggaccagagatagtcattgaacacataaaagggaagagcaatggtttagctgacatcttgtccaggctcaaagccaaattagctcagaatgaaccaacggaagagatgatcctgcttacacaagccataagggaagtaattccttatccagatcatccatacactgagcaactcagagaatggggaaacaaaattctggatccattccccacattcaagaaggacatgttcgaaagaacagagcaagcttttatgctaacagaggaaccagttctactctgtgcatgcaggaagcctgcaattcagttagtgtccagaacatctgccaacccaggaaggaaattcttcaagtgcgcaatgaacaaatgccattgctggtactgggcagatctcattgaagaacacattcaagacagaattgatgaatttctcaagaatcttgaagttctgaagaccggtggcgtgcaaacaatggaggaggaacttatgaaggaagtcaccaagctgaagatagaagagcaggagttcgaggaataccaggccacaccaagggctatgtcgccagtagccgcagaagatgtgctagatctccaagacgtaagcaatgacgattgaggaggcattgacgtcagggatgaccgcagcggagagtactgggcccattcagtggatgctccactgagttgtattattgtgtgcttttcggacaagtgtgctgtccactttcttttggcacctgtgccactttattccttgtctgccacgatgcctttgcttagcttgtaagcaaggatcgcagtgcgtgtgtgacaccaccccccttccgacgctctgcctatataaggcaccgtctgtaagctcttacgatcatcggtagttcacca

SEQ ID NO: 28 - DNA, 300 bp, Sugarcane bacilliform virus
agatctccaagacgtaagcaatgacgattgaggaggcattgacgtcagggatgaccgcagcggagagtactgggcccattcagtggatgctccactgagttgtattattgtgtgcttttcggacaagtgtgctgtccactttcttttggcacctgtgccactttattccttgtctgccacgatgcctttgcttagcttgtaagcaaggatcgcagtgcgtgtgtgacaccaccccccttccgacgctctgcctatataaggcaccgtctgtaagctcttacgatcatcggtagttcacca

SEQ ID NO: 29 - DNA, 253 bp, Agrobacterium tumefaciens
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 30 - DNA, 1010 bp, Zea mays
gtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 31 - DNA, 583 bp, Orzya sativa
gtaagatccgatcaccatcttctgaatttctgttcttgatctgtcatgtataataactgtctagtcttggtgttggtgagatggaaattcggtggatctcggaagggatattgttcgtttgctggggttttttttgtgtgttgtgatccgtagagaatttgtgtttatccatgttgttgatcttggtatgtattcatgacatattgacatgcatgtgttgtatgtgtcatatgtgtgcctctccttgggatttgttttggataatagaacatgttatggactcaatagtctgtgaacaaatctttttttagatggtggccaaatctgatgatgatctttcttgagaggaaaaagttcatgatagaaaaatcttttttgagatggtggcttaatgtgatgatgatctttcttgagaggaaaaaaaagattcattataggagattttgatttagctcctttccaccgatattaaatgaggagcatgcatgctgattgctgataaggatctgatttttttatcccctcttctttgaacagacaagaaataggctctgaatttctgattgattatttgtacatgcag

SEQ ID NO: 32 - DNA, 36 bp, Artificial Sequence
  Bm3d
ctgtcgaggatgcatgtcgtggattccaacaagcca

SEQ ID NO: 33 - DNA, 36 bp, Artificial Sequence
  Bm3d
ccgcggcttagaatccacgacatgcatccacgactc

SEQ ID NO: 34 - DNA, 36 bp, Artificial Sequence
  Bm3e
ctgtcgacgtacgacgcgctgccggacaacaagcca

SEQ ID NO: 35 - DNA, 36 bp, Artificial Sequence
  Bm3e
ccgcggcttttccggcagcgcgtcgtacgacgactc

SEQ ID NO: 36 - DNA, 102 bp, Artificial Sequence
  insert sequence for detecting Bm3d miRNA by 3′ adaptor method
gagaagcttgtcgacgaattcagattttagaatccacgacatgcatcatgcggtggtggctgagcgggctggcaaggcaatcacgaattctggatccgatac

SEQ ID NO: 37 - DNA, 31 bp, Artificial Sequence
  R5 3′ Adaptor, R5 oligo
augcggtggtggctgagcgggctggcaaggc

SEQ ID NO: 38 - DNA, 19 bp, Artificial Sequence
  primer
gccttgccagcccgctcag

SEQ ID NO: 39 - DNA, 18 bp, Artificial Sequence
  primer
ttagaatccacgacatgc

SEQ ID NO: 40 - DNA, 21 bp, Artificial Sequence
  Nucleotide sequence of ABI Bm3d for the ABI assay to detect the Bm3d artificial miRNA
ttagaatccacgacatgcatc

SEQ ID NO: 41 - DNA, 31 bp, Artificial Sequence
  primer
gccttaattaaggagatgatctcaggctatc

SEQ ID NO: 42 - DNA, 31 bp, Artificial Sequence
  primer
taaggcgcgccatcgaggagcgcgctagctg

SEQ ID NO: 43 - DNA, 31 bp, Artificial Sequence; identical to SEQ ID NO: 41
  primer

SEQ ID NO: 44 - DNA, 31 bp, Artificial Sequence; identical to SEQ ID NO: 42
  primer

SEQ ID NO: 45 - DNA, 21 bp, Artificial sequence
  miRNA miRBm3d
ttagaatccacgacatgcaag

SEQ ID NO: 46 - DNA, 21 bp, Artificial Sequence
  miRNA miRBm3e
ttttccggcagcgcgtcgtag

SEQ ID NO: 47 - DNA, 175 bp, Artificial Sequence
  Zm_pre_miR166 variant
gcagcgtcggtggttgcttcatggctgtcgaggggaatgacgtccggtccgaacaagccacggctgctgctgcgctaccgccgcggcttcggaccaggcttcattccccacgactcaccatggaaacggcagcgaggtgcgagcttccagtgcgcgcagtgtttgatcagcaaac

SEQ ID NO: 48 - DNA, 693 bp, Artificial Sequence
  Zm_pre_miR166 variant
ctgacgctgctgccggagacctactctgctactggtgtggagtggacaaacagggagggaaggcgtctgctagaacctagaagacggacacagggaggaccaacaggactaaagaccgtcgtacgtgttgaagcggaaggactttcttattacgtgtggcgcttcgaattaaagccagcggttagaacggcgcagcgtcggtggttgcttcatggctgtcgaggggaatgacgtccggtccgaacaagccacggctgctgctgcgctaccgccgcggcttcggaccaggcttcattccccacgactcaccatggaaacggcagcgaggtgcgagcttccagtgcgcgcagtgtttgatcagcaaacacctgcgtggccgaggctgagccttgcaagggcaggcgtccatgttctcgtcagcaagcgtcttcttcacatgtctgaggtctgagcaattgcgggtggacatcgatggatgagcattgccgctgcaagggaagatcgtccggctcacacttgagttgggataccatgcactactcttgttgtagttagattagataccatgcgcttgaaccgtgcaatagcatagcgaggaagaggaacccagagaggactcagaaagagatgttgtctatttcatccagtgctcatctagtcatctagtctgcctgcgggtgtctgcaatgaacg

SEQ ID NO: 49 - DNA, 374 bp, Artificial Sequence
  Zm_pre_miR166 variant
ctaccgccgcggcttcggaccaggcttcattccccacgactcaccatggaaacggcagcgaggtgcgagcttccagtgcgcgcagtgtttgatcagcaaacacctgcgtggccgaggctgagccttgcaagggcaggcgtccatgttctcgtcagcaagcgtcttcttcacatgtctgaggtctgagcaattgcgggtggacatcgatggatgagcattgccgctgcaagggaagatcgtccggctcacacttgagttgggataccatgcactactcttgttgtagttagattagataccgcagcgtcggtggttgcttcatggctgtcgaggggaatgacgtccggtccgaacaagccacggctgctgctgcg

SEQ ID NO: 50 - DNA, 1035 bp, Oryza sativa
  promoter (1)..(1035): transcription regulating sequence of caffeoyl CoA-O-methyltransferase gene including 5′-untranslated region
gcaactactgcacggtaaaagtgataggaatcggtcggaaacagtattaatgtttttattatttttacaaaaacgaattgaaataattggaaattttcatatttatatattaaactattcagtatcaacttcaattcgacgtcaatagaaattagaaaagcataattatacacagtaataggcgttcaagatattattgttattatttagttttgtggaaatggtatcaacgtgatcggaaaattttgtacatgttttcaccctgcgggatatctcaattccttctcctccctctaccgccatatcagcacacgttttagagcaccaatcataacccataaatccgtgggctactcacttatttaatttatatgtgaattcgtgacctgactcactcacatactatcaaaaatttgtctcagtcacccatctccttctttcctggtccgataagggtttatcctacggttcgacggttatcacgatagtcgtgcggttactgaggtataccgtgatttaaaaatatgataaagttaccgcaggttttaactgcgcggtttggtaaacctgttcctcctcaccaaccttctcctccggtctccttatgtgtctcaccgaggcgagccgccgcgagaccgcatggacgcggtccacgcacctggcggtgcacctcctcctccccggcgaagaagacgtggaggagagtaaatgagcaatcaggcccacggcccaatcgccgtccaccacccaccaccctcagcgacccaaaaccacctcaccaacccaactctgtaccgtactgtacccgccctcccctcccactgacactccgggcccacctgtcggcgcgactcttccacggtccccttctctcctcagagattttttccacgcattttttaatttttttttctgcagttcacatgctcttctcccactcttccgccgcgctatataaaccgcgcgaggcgtcgttgcctcgtcggcgaagtcaatccggcgatccccggcgagcgagagatcgaagcaagctgc

(19) United States
(12) Patent Application Publication
(10) Pub. No.: US 2012/0272406 A1
(43) Pub. Date: Oct. 25, 2012

(54) METHODS OF MODIFYING LIGNIN BIOSYNTHESIS AND IMPROVING DIGESTIBILITY

(75) Inventors: Hanping Guan, Chapel Hill, NC (US); Beomseok Seo, Morrisville, NC (US); Peifeng Ren, Cary, NC (US); David Deppong, Cary, NC (US); Huihua Fu, Cary, NC (US)

(73) Assignee: BASF Plant Science Company GmbH, Ludwigshafen (DE)

(21) Appl. No.: 13/434,035

(22) Filed: Mar. 29, 2012

Related U.S. Application Data
(60) Provisional application No. 61/477,916, filed on Apr. 21, 2011.
(60) Provisional application No. 61/602,813, filed on Feb. 24, 2012.

(51) Int. Cl. C12N 15/82; C12N 5/10; C12N 1/13; C12N 1/21; C12N 1/15; C12N 1/19; C12N 15/113; A01H 5/00

(52) U.S. Cl. 800/285; 435/320.1; 536/24.1; 435/419; 800/298; 435/252.33; 435/252.3; 435/252.31; 435/252.2; 435/254.8; 435/254.2; 435/257.2; 435/254.3; 435/254.6; 435/254.11; 435/254.4; 435/254.7; 435/254.22; 435/254.21; 435/254.23; 800/278; 435/468

(57) ABSTRACT

The present invention relates to methods for increasing digestibility and/or crude protein of a plant by modifying lignin biosynthesis of the plant. The methods involve the manipulation of the expression level of genes in the lignin biosynthesis pathway by directly reducing the expression of genes using miRNA or using regulation of transcription factors. Expression cassettes for achieving such gene expression manipulation and transgenic plant cells and plants comprising the constructs and cassettes are also provided. Transcription regulating nucleotide sequences for use in the expression cassettes have also been developed as well as methods of using these sequences and cassettes.

### CROSS REFERENCE TO RELATED APPLICATIONS

The present invention claims the priority benefit of U.S. Provisional Patent Application Ser. No. 61/477,916 filed Apr. 21, 2011 and U.S. Provisional Patent Application Ser. No. 61/602,813 filed Feb. 24, 2012, the entire contents of each are hereby incorporated by reference in their entirety.

### SUBMISSION OF SEQUENCE LISTING

The Sequence Listing associated with this application is filed in electronic format via EFS-Web and hereby incorporated by reference into the specification in its entirety. The name of the text file containing the Sequence Listing is Sequence_Listing_{—}17731_{—}00035_US. The size of the text file is 47 KB, and the text file was created on Mar. 29, 2012.

### FIELD OF THE INVENTION

This invention relates generally to methods for increasing digestibility and/or crude protein of a plant by modifying lignin biosynthesis of the plant. The methods involve the manipulation of the expression level of genes in the lignin biosynthesis pathway by directly reducing the expression of genes using miRNA or using regulation of transcription factors. Expression cassettes for achieving such gene expression manipulation are also provided.

### BACKGROUND

Cereal crops like corn, rice, wheat, oats, and rye are a major source of nutrients for many types of livestock and supply most of their dietary energy needs. Fiber is a major component of these plants, making up 25 to 80% of their dry weight. Fiber is composed mainly of complex carbohydrates that are potentially an important source of digestible energy for livestock. Unfortunately, the enzymatic breakdown of complex carbohydrates into sugars is limited by an indigestible component in fiber known as lignin (Grabber, Crop Science, 2005, 45:820-831). Lignin is present in all vascular plants and is an essential constituent of cell walls. It supports the structural integrity of the cell wall and enhances stiffness and strength of the stem. In addition, lignin plays key roles in enabling fluid transport through the vascular system and conferring resistance to pathogens and mechanical stress. Lignins also affect the digestibility of plant cell wall biomass (Hatfield et al., Crop Science, 1999, 39:27-37; U.S. Pat. No. 7,453,023) and it has been demonstrated that reducing the amount of lignin results in an overall increase in cell wall digestibility (Grabber, Crop Science, 2005, 45:820-831). Therefore, lignin modification is especially important in plant material used for feed silage and forage. Lignin modification is also important in plant material used for pulping and biofuel. Downregulation of lignin biosynthetic enzymes in alfalfa has been shown to increase total carbohydrate levels and sugar release by enzymatic hydrolysis (Chen et al., Nature Biotech., 2007, 25:759-761). Therefore, altering lignin content may increase the carbohydrates available for biofuel production. Due to these properties, numerous research efforts have been made to modify lignin content and/or composition.

The lignin biosynthetic pathway has been well characterized in several plant species (Humphreys and Chapple, Current Opinion in Plant Biology, 2002, 5:224-229; Boerjan et al., Annual Review in Plant Biology, 2003, 54:519-546; Barriere et al., Gene, Genomes, and Genomics, 2007, 1:133-156; Davin et al., Natural Products Reports, 2008, 25:1015-1090). A general overview of the lignin biosynthetic pathway is shown in FIG. 1. The lignin biosynthesis pathway begins after the shikimate pathway with the deamination of L-Phe into cinnamic acid. Successive steps of hydroxylation and methylation on the aromatic ring lead to production of three monolignols, p-coumaryl, coniferyl, and sinapyl alcohols. These monolignols are polymerized into three distinct lignins, resulting in p-hydroxyphenyl (H), guaiacyl (G) and syringyl (S) units, respectively. The enzymes from this pathway include phenylalanine ammonia lyase (PAL), cinnamate 4-hydrolase (C4H), 4-coumarate:CoA ligase (4CL), hydroxycinnamoyl-CoA:shikimate and quinate hydroxyl-cinnamoyl transferase (HCT), p-coumarate 3-hydroxylase (C3H), caffeoyl-CoA 3-O-methyltransferase (CCoAOMT), cinnamoyl-CoA reductase (CCR), cinnamyl alcohol dehydrogenase (CAD), sinapyl alcohol dehydrogenase (SAD), ferulate 5-hydroxylase (F5H), caffeic acid O-methyltransferase (COMT), and hydroxycinnamaldehyde dehydrogenase (HCALDH), peroxidise, and laccase.

Manipulation of lignin biosynthesis genes provides one potential method for modifying lignin content. A natural maize mutant containing a loss of function mutation in the lignin biosynthesis gene COMT has been isolated and is termed brown mid-rib 3 (bm3). Vignols et al. (Plant Cell, 1995, 7:407-416) and Morrow et al. (Molecular Breeding, 1997, 3:351-357) established that bm3 mutants resulted from insertion or deletion mutations in the COMT gene. Guillaumie et al. (BMC Plant Biology, 2008, 8:71-86) reported that the lignin content of maize bm3 mutants was reduced by approximately 24-40% and p-coumaric acid (pCA) was reduced by about 50%. The cell wall digestibility of bm3 genotypes was increased by about 9%, compared with isogenic hybrids.

The bm3 mutation has also been shown to improve fiber digestibility by dairy cows and improve silage quality. However, this mutation also causes reduced biomass yield, thus limiting its value per acre. Another disadvantage of the natural mutant is that it is recessive, thus both parents must have the mutant allele to make corn hybrids exhibiting the bm3 phenotype. Therefore, despite its improved silage quality, the bm3 mutant has limited applicability for the development of commercial maize lines.

Because of the limited applicability of the bm3 mutant, transgenic approaches have been attempted to develop plants with improved silage quality but without undesirable agronomic traits. Several studies altering lignins through genetic manipulation and mutation of lignin biosynthetic genes have been reported (Anterola and Lewis, Phytochemistry, 2002, 61:221-294; Vanholme et al., Current Opinion in Plant Biology, 2008, 11:278-285), one example of which is the manipulation of COMT gene.

The maize COMT gene (ZmCOMT) has been isolated (Collazo et al., Plant Molecular Biology, 1992, 20:857-867) and several plant COMT homologs have been disclosed (U.S. Pat. No. 7,655,448). In the maize lignin biosynthetic pathway, ZmCOMT is involved in methylation of 5-hydroxy-coniferaldehyde to form sinapaldehyde, which is the precursor of sinapyl alcohol. Several reports demonstrated that down-regulation of COMT activity resulted in reduced lignin content and/or increased bioavailability of cell wall fiber in maize (Piquemal et al., Plant Physiology, 2002, 130:1675-1685; He et al., Crop Science, 2003, 43:2240-2251), tobacco (Vailhe et al., Journal of the Science of Food and Agriculture, 1996, 72:385-391), Arabidopsis (Goujon et al., Plant Molecular Biology, 2003, 51:973-989), poplar (Jouanin et al., Plant Physiology, 2000, 123:1363-1373; WO 1993/05160), alfalfa (Guo et al., Plant Cell, 2001, 13:73-88; Guo et al., Transgenic Research, 2001, 10:457-464; WO 1994/23044; WO 2001/73090; U.S. 2007/0079398), and eucalyptus (U.S. 2005/0026162).

In maize, for example, down-regulation of COMT through antisense expression of maize COMT cDNA under the control of the maize Adh1 promoter resulted in characteristics similar to those observed in maize bm3 mutants (Piquemal et al., Plant Physiology, 2002, 130:1675-1685; WO 03/018819). Similarly, down-regulation of COMT by expressing antisense sorghum COMT cDNA under the maize ubiquitin-1 (Ubi-1) promoter (plus the first intron and exon of Ubi-1) reduced lignin content in T1 transgenic plants by an average of 17% on a whole plant basis and increased mean whole-plant in vitro NDF digestibility (IVNDFD) from 72% to 76% (He et al., Crop Science, 2003, 43:2240-2251). Likewise, down-regulation of COMT activity in corn cobs by RNAi increased IVNDFD by 4.1% to 7.3% (WO 2006/104891).

Despite the advances made in genetic manipulation of COMT expression, no transgenic commercial product is on the corn silage market today, possibly hampered by negative agronomic traits and yield reduction. As demonstrated by the bm3 corn mutant, cell wall modification such as reduction of lignin content can cause unfavorable agronomic traits such as abnormal morphology, susceptibility to lodging and disease, and biomass/yield loss. In an effort to improve cell wall degradability or digestibility, it is thus important to find a way to regulate the timing, location, and/or level of lignin biosynthesis gene expression to overcome such unfavorable traits.

There are several technologies available for silencing or down-regulating gene expression in plants including conventional antisense RNA, sense RNA for co-suppression, double stranded RNA (dsRNA), small interfering RNA (siRNA), and microRNA (miRNA). MicroRNAs have emerged as evolutionarily conserved, RNA-based regulators of gene expression in animals and plants. MicroRNAs (approx. 18 to 25 nt) arise from larger precursors, pre-miRNAs, with a stem loop structure that are transcribed from non-protein-coding genes. The miRNAs appear to be transcribed as hairpin RNA precursors, which are processed to their mature (about 21 nt) forms by Dicer (Lee R D, et al., Science, 2001, 294:862-864). miRNA targets a specific mRNA to suppress gene expression at the post-transcriptional level (i.e. by degrading mRNA) or at the translational level (i.e. by inhibiting protein synthesis). Gene expression-repressing plant microRNAs often contain near-perfect complementarity with target sites, which occur most commonly in protein-coding regions of mRNAs (Llave C et al., Science 2002, 297:2053-2056; Rhoades M W et al., Cell, 2002 110:513-520). However, Schwab et al. (Developmental Cell, 2005, 8:517-527) have shown that miRNAs can still target genes with 4 or even 5 mismatches with the target gene.

In plants most gene expression-repressing plant microRNAs function to guide target RNA cleavage (Jones-Rhoades M W et al., Mol. Cell, 2004, 14:787-799; Kasschau K D et al., Dev. Cell, 2003, 4:205-217). Various publications describe the function of microRNAs and their use as tool for downregulation of target gene expression by overexpression of endogenous or recombinant microRNAs in plants. Ossowski et al. (Plant J., 2008, 53:674-690) provide an overview on methods for gene silencing using artificial siRNAs such as microRNAs.

Schwab et al. (Developmental Cell, 2005, 8:517-527) have also shown that in Arabidopsis that not all microRNA precursors work equally well for silencing when engineered for repression of target genes. For example, precursors MIR319a and MIR172a engineered for targeting the same target gene show different degree of downregulation of the target gene, the MIR319a being more efficient in silencing the respective target gene. Moreover, a microRNA precursor well suited for efficient target gene silencing in one species may not work equally well in another species (Alvarez J P et al., Plant Cell, 2006, 18:1134-1151).

MicroRNA technology has not been developed for regulating lignin biosynthesis gene expression.

Another possibility is to use the promoter of a gene from the lignin biosynthetic pathway so that the desired down-regulation occurs only in lignifying cells. However, the use of a lignin biosynthesis gene promoter to regulate lignin production can have negative effects on plant development. For example, use of the bean PAL promoter to down-regulate lignin biosynthesis in cell walls has been shown to have undesirable effects on plant morphology, growth, and biomass (Reddy et al., PNAS, 2005, 102:16573-16578).

A promoter region of the maize COMT gene has been previously isolated. Capellades et al. (Plant Mol. Biol., 1996, 31:307-322) reported the isolation of a 1963 bp Sph1-Xho1 DNA promoter fragment of the maize COMT gene, ranging from position −1955 of the putative start of transcription to the +7 position. This promoter fragment was isolated from a maize inbred W64A genomic library in an attempt to isolate the COMT coding sequence (Collazo et al., Plant Mol. Biol., 1992, 20:857-867). A construct comprising the β-glucuronidase (GUS) gene under transcriptional control of this COMT promoter was transformed into maize and tobacco. GUS expression pattern observed in 15-20 day old maize plants corresponded to the expression pattern of the endogenous COMT gene, i.e. this 1963-bp COMT promoter was active in tissues undergoing lignification such as the small vascular strands of young leaves and the vascular bundles of stems (Capellades, Plant Mol. Biol., 1996, 31:307-322).

In an effort to identify genetic diversity associated with variation in silage corn digestibility, the promoter sequences up to 1.1 kb of the COMT gene from 34 maize lines, including the inbred line W64A, were isolated (Genbank Accession No. AY323283) and the promoter elements were identified. These promoter elements included the transcription initiation site (+1) found 147 bp upstream of the translation start codon ATG, a TATA box (−28), BoxA (−41), BoxIV (−188), MRE (−205), Maize P (−666), BoxP (−808) and MYB.Ph3 (−864) (Guillet-Claude et al., Theor. Appl. Genet., 2004, 110:126-135).

Although a COMT promoter with the same expression pattern as the endogenous gene may be useful for cell/tissue specific expression, this expression pattern may not be desirable for the regulation of lignin biosynthesis in transgenic plants. For example, if this promoter were used to downregulate COMT expression to the level found in the bm3 mutants, the negative properties of the bm3 mutants would also be likely produced. Therefore, there is still a need for a promoter that could direct transgene expression in lignifying tissues, but would avoid producing the negative agronomic traits observed in the bm3 mutants.

In addition to manipulating lignin biosynthesis through direct down-regulation of lignin biosynthesis gene expression, another potential method in regulating lignin biosynthesis is through the manipulation of transcription factor expression: MYB, MADS-box, bHLH, KNOX and LIM transcription factors have been shown to be involved in the regulation of lignin biosynthesis (Tamagnone et al., Plant Cell, 1998, 10:135-154; Mele et al., Genes & Dev., 2003, 17:2088-2093; Zhong and Ye, Current Opinion in Plant Biol., 2007, 10:564-572; Zhou et al., Plant Cell, 2009, 21:248-266; Sonbol et al., Plant Mol. Biol., 2009, 70:283-296; U.S. 2009/0070899). Transcription factors containing the MYB domain belong to a large multigene family, with 126 members in Arabidopsis thaliana, more than 200 members in Zea mays, and 183 members in Oryza sativa (Sonbol et al., Plant Mol. Biol., 2009, 70:283-296). Several MYB transcription factors, specifically the R2R3-MYB transcription factors, have been shown to associate with the down-regulation of lignin biosynthesis in Antirrhinum majus (Tamagnone et al., Plant Cell, 1998, 10:135-154), Arabidopsis thaliana (Jin et al., EMBO J., 2000, 19:6150-6161), Eucalyptus gunnii (Legacy et al., Plant Sci., 2007, 173:542-549), Pinus taeda (Patzlaff et al., Plant J., 2003, 36:743-754), and Zea mays (Formale et al., Plant Mol. Biol., 2006, 62:809-823).

In maize, the transcription factors ZmMYB31 and ZmMYB42 were identified as R2R3-MYB transcription factors capable of down-regulating lignin biosynthesis gene expression. For example, overexpression of ZmMYB31 in Arabidopsis reduced 4CL and COMT expression and slightly increased CAD expression, and the transgenic plants exhibited reduced lignin content by 87% (Formalé et al., Plant Mol. Biol., 2006, 62:809-823). Likewise, it is demonstrated that overexpression of ZmMYB42 in Arabidopsis down-regulated several genes of the lignin biosynthesis pathway, including PAL1, C4H, 4CL1, HCT, F5H1, CAD6 and COMT1, which resulted in reduced lignin content by 53% (Formalé et al., Plant Mol. Biol., 2006, 62:809-823; Sonbol et al., Plant Mol. Biol., 2009, 70:283-296). In these demonstrations, ZmMYB42 was overexpressed under the control of the double CMV 35S promoter and the pA35S transcription terminator (Formale et al., Plant Mol. Biol., 2006, 62:809-823). However, although containing reduced lignin content as compared to wild type plants, the transgenic plants were reported to be smaller than wild type plants and had abnormal cell wall structure (Sonbol et al., Plant Mol. Biol., 2009, 70:283-296). Thus, there is still a need to develop appropriate transgenic expression systems for manipulating the expression of the transcription factor ZmMYB42 to reduce the negative effects on agronomic traits and yield while retaining its ability to reduce lignin content in plants.

### BRIEF SUMMARY

The present invention provides novel expression cassettes and methods for increasing digestibility and/or crude protein of a plant. Recombinant constructs, vectors, and plant cells, plants or parts thereof, comprising the expression cassettes of the invention as well as methods for their production are also provided.

In one aspect, the invention provides an expression cassette for down-regulating lignin biosynthesis in a plant comprising:
(a) a transcription regulating nucleotide sequence of a Caffeoyl-CoA O-methyltransferase (CCoAOMT) gene, and
(b) a nucleic acid sequence encoding the maize transcription factor MYB42 (ZmMYB42) operably linked to said transcription regulating nucleotide sequence.

In further embodiments, the transcription regulating nucleotide sequence of a CCoAOMT is from a rice CCoAOMT (OsCCoAOMT) gene. In another embodiment, the transcription regulating nucleotide sequence of a CCoAOMT gene comprises the polynucleotide sequence of SEQ ID NO: 7. The gene encoding the maize transcription factor MYB42 (ZmMYB42) may comprise a polynucleotide sequence selected from the group consisting of:
(a) the polynucleotide sequence of SEQ ID NO: 1,
(b) a polynucleotide sequence encoding a polypeptide having the amino acid sequence of SEQ ID NO: 2, and
(c) a polynucleotide sequence encoding a polypeptide having at least 95% identity to SEQ ID NO: 2 and having activity of a transcription factor.

In another aspect, the invention provides an expression cassette comprising:
(a) a transcription regulating nucleotide sequence, and operably linked thereto
(b) a miR166 precursor sequence that is engineered to produce a miRNA sequence which reduces expression of a gene in the lignin biosynthesis pathway.

Prior to engineering, the miR166 precursor sequence may comprise a polynucleotide sequence selected from the group consisting of:
(a) the polynucleotide sequence of SEQ ID NO: 16,
(b) a polynucleotide sequence having at least 75% sequence identity with the sequence of SEQ ID NO: 16 that is capable of producing a miRNA sequence, and
(c) a functional fragment of the polynucleotide sequence of SEQ ID NO: 16 that is capable of producing a miRNA sequence.

In one embodiment, the polynucleotide sequence comprises a sequence having at least 90% sequence identity with the sequence of SEQ ID NO: 16. In a further embodiment, variants of the miR166 precursor comprise SEQ ID NO: 47, SEQ ID NO: 48, or SEQ ID NO: 49.

In specific embodiments, the miR166 precursor sequence is engineered by replacing a first segment of about 19-24 contiguous nucleotides located between positions corresponding to about nucleotide 32 and nucleotide 55 of SEQ ID NO: 16 with a first nucleotide sequence of about 19-24 nucleotides, and a second segment of about 19-24 contiguous nucleotides located between positions corresponding to about nucleotide 86 and nucleotide 109 of SEQ ID NO: 16 with a second nucleotide sequence of about 19-24 nucleotides, wherein the first nucleotide sequence and the second nucleotide sequence are substantially complementary to each other, and the second nucleotide sequence is substantially complementary to a portion of a mRNA transcribed from a gene in the lignin biosynthesis pathway. In yet another embodiment, the first segment comprises the polynucleotide sequence of SEQ ID NO: 17 and the second segment comprises the polynucleotide sequence of SEQ ID NO: 18. In a further embodiment, the second nucleotide sequence is substantially complementary to the 5′-UTR, 3′-UTR, or coding region of a mRNA transcribed from the gene in the lignin biosynthesis pathway.

The gene in the lignin biosynthesis pathway, of which the expression is to be reduced by the miRNA sequence produced by the miR166 precursor, preferably encodes a polypeptide selected from the group consisting of phenylalanine ammonia lyase (PAL), cinnamate 4-hydrolase (C4H), 4-coumarate:CoA ligase (4CL), hydroxycinnamoyl-CoA:shikimate and quinate hydroxyl-cinnamoyl transferase (HCT), p-coumarate 3-hydroxylase (C3H), caffeoyl-CoA 3-O-methyltransferase (CCoAOMT), cinnamoyl-CoA reductase (CCR), hydroxycinnamaldehyde dehydrogenase (HCALDH), cinnamyl alcohol dehydrogenase (CAD), sinapyl alcohol dehydrogenase (SAD), ferulate 5-hydroxylase (F5H), caffeic acid O-methyltransferase (COMT), laccase, and peroxidase. In preferred embodiments, the gene in the lignin biosynthesis pathway is a caffeic acid O-methyltransferase (COMT) gene. In another embodiment, the COMT gene is a maize COMT (ZmCOMT) gene.

In still another embodiment, the miR166 precursor sequence present in the expression cassette of the invention is operably linked to a transcription regulating nucleotide sequence that is a constitutive promoter, a tissue-specific or tissue-preferential promoter, an inducible promoter, or a developmentally regulated promoter. In one embodiment, the transcription regulating nucleotide sequence is from a COMT gene. In another embodiment, the transcription regulating nucleotide sequence is from a maize COMT (ZmCOMT) gene. In a further embodiment, the transcription regulating nucleotide sequence comprises the polynucleotide sequence of SEQ ID NO: 13.

In a further embodiment, the first nucleotide sequence to be used to replace the first segment of the miR166 precursor sequence comprises:
(a) the polynucleotide sequence of SEQ ID NO: 19 or SEQ ID NO: 21, or
(b) a polynucleotide sequence having at least 70% sequence identity to the polynucleotide sequence of SEQ ID NO: 19 or SEQ ID NO: 21,
(c) and the second nucleotide sequence to be used to replace the second segment of the miR166 precursor sequence comprises:

the polynucleotide sequence of SEQ ID NO: 20 or SEQ ID NO: 22, or
(a) a polynucleotide sequence having at least 70% sequence identity to the polynucleotide sequence of SEQ ID NO: 20 or SEQ ID NO: 22,
(b) wherein the first nucleotide sequence and the second nucleotide sequence remain substantially complementary to each other, and the second nucleotide sequence is capable of reducing expression of a maize COMT gene.

In one embodiment, the miR166 precursor sequence is engineered to contain the polynucleotide sequence of SEQ ID NO: 23, SEQ ID NO: 24, or SEQ ID NO: 25.

In a further embodiment, the aforementioned expression cassette comprises a second miR166 precursor sequence, in which the second miR166 precursor sequence is engineered to produce a second miRNA sequence which reduces expression of a gene in the lignin biosynthesis pathway. In a specific embodiment, the expression cassette having two miR166 precursor sequences comprises the polynucleotide sequence of SEQ ID NO: 26.

In yet another aspect, the invention provides a transcription regulating nucleotide sequence comprising:
(a) the polynucleotide sequence of SEQ ID NO: 13,
(b) a polynucleotide sequence comprising at least 750 nucleotides and having at least 90% identity to the nucleotide sequence of SEQ ID NO: 13, wherein said nucleotide sequence is capable of driving expression of a heterologous nucleic acid sequence that is operably linked thereto, or

(c) a fragment of SEQ ID NO: 13, wherein the fragment comprises at least 750 nucleotides and is capable of driving expression of a heterologous nucleic acid sequence that is operably linked thereto.

In another embodiment, the invention further provides an expression cassette comprising the immediately aforementioned transcription regulating nucleotide sequence operably linked to a heterologous nucleic acid sequence. In yet another embodiment, the expression of the operably linked heterologous nucleic acid sequence results in expression of a protein, or expression of an antisense RNA, a sense RNA, a double-stranded RNA, or a miRNA. In a further embodiment, the expression of the operably linked heterologous nucleic acid sequence confers to the plant an agronomically valuable trait. In yet a further embodiment, the agronomically valuable trait conferred by the expression of the operably linked heterologous nucleic acid sequence is reduced lignin production, increased crude protein, and/or increased cell wall digestibility.

In particular embodiments, any of the aforementioned expression cassettes further comprises a terminator, an enhancer, or a terminator and an enhancer. In one embodiment, the terminator is from a CCoAOMT gene. The terminator may be from a rice CCoAOMT (OsCCoAOMT) gene. In an alternative embodiment, the terminator comprises the polynucleotide sequence of SEQ ID NO: 10. In a further embodiment, the enhancer is an intron, preferably the polynucleotide sequence of SEQ ID NO: 31.

In a further embodiment, the invention provides a recombinant construct comprising any of the aforementioned expression cassettes. The recombinant construct of the invention may comprise at least two of the aforementioned expression cassettes. The invention further provides vectors comprising any of the aforementioned recombinant constructs.

The invention also provides a plant cell, plant or part thereof, or microorganism comprising at least one of the aforementioned expression cassettes, a recombinant construct comprising at least one of the aforementioned expression cassettes, or a vector comprising at least one of the aforementioned expression cassettes or the aforementioned recombinant constructs.

Further, the invention provides a method for producing a transgenic plant or plant cell, comprising:
(a) transforming a plant or plant cell with at least one of the aforementioned expression cassettes, a recombinant construct comprising at least one of the aforementioned expression cassettes, or a vector comprising at least one of the aforementioned expression cassettes or the aforementioned recombinant constructs, and
(b) optionally regenerating from the plant cell a transgenic plant.

In still another aspect, the invention provides a method for increasing digestibility of a plant, comprising:
(a) transforming a plant or a plant cell with at least one of the aforementioned expression cassettes, a recombinant construct comprising at least one of the aforementioned expression cassettes, or a vector comprising at least one of the aforementioned expression cassettes or the aforementioned recombinant construct,
(b) growing said transformed plant or plant cell,
(c) optionally, regenerating from the plant cell a transgenic plant,
(d) wherein expression of the nucleic acid sequence and/or the miR166 precursor sequence comprised in the aforementioned expression cassette results in down-regulation of lignin biosynthesis in the plant or plant cell and confers an increase in digestibility and/or crude protein of the transgenic plant or part thereof as compared to a corresponding wild-type plant. The plant or parts thereof may be processed into feed, e.g., for livestock.

The plant used in the aforementioned method may be a monocotyledonous plant, or the plant cell or plant part used for transformation is from a monocotyledonous plant. In a preferred embodiment, the plant used in the aforementioned method is a maize plant, or the plant cell or plant part used for transformation is from a maize plant.

The invention also concerns the use of the plant, or parts thereof, produced by the aforementioned methods for the production of foodstuff, feedstuff, food supplement, or feed supplement. In another embodiment, the invention further provides methods for the preparation of a composition intended for animal or livestock feed comprising the silage of the plant produced by the aforementioned methods.

### DESCRIPTION OF THE FIGURES

FIG. 1 shows an overview of the lignin biosynthetic pathway with lignin altering enzymes.

FIG. 2A-C shows an alignment between the ZmCOMT promoter described herein (SEQ ID NO: 13) and the ZmCOMT promoter sequence previously isolated from the inbred line W64A (including the 5′UTR of W64A COMT genomic DNA of GenBank Accession No. AY323283; SEQ ID NO: 14). Identical nucleotides are shown in white text with black background.

FIG. 3 depicts the fold-back alignments of artificial miRNAs Bm3d (A.; top SEQ ID NO: 32; bottom SEQ ID NO: 33) and Bm3e (B.; top SEQ ID NO: 34; bottom SEQ ID NO: 35). The miRNA sequence is represented in bold italics and aligned at the bottom and the corresponding star sequence is in bold and aligned at the top. Intentional mismatches between the miRNA and Star are underlined. The sequence that is flanking the miRNA and Star sequences are part of the maize miR166 pre-miRNA backbone. The Bm3d/3e double stack is shown in C.

FIG. 4 shows the insert sequence in detecting Bm3d miRNA by 3′ adaptor method. The sequence was confirmed by sequencing inserts clones generated in Example 4.1. The BM3Dminus3 primer annealing site is in bold font, the three bases at the 3′ end of the artificial Bm3d miRNA are in italics, and the R53′ adaptor is underlined. Flanking sequences are from the Qiagen pDrive vector. The full sequence of the artificial Bm3d miRNA is therefore a combination of the bold and italic font.

FIG. 5 shows an alignment of polynucleotide sequences AM156908 from W64A (Formale et al., Plant Mol. Biol., 2006, 62:809-823; SEQ ID NO: 3) and ZmMYB42 from B73 (SEQ ID NO: 1).

FIG. 6 shows an alignment of the protein sequences translated from the polynucleotide sequences AM156908 from W64A (Formale et al., Plant Mol. Biol., 2006, 62:809-823; SEQ ID NO: 4) and ZmMYB42 from B73 (SEQ ID NO: 2), respectively.

FIGS. 7A-L depict the various sequences described herein.

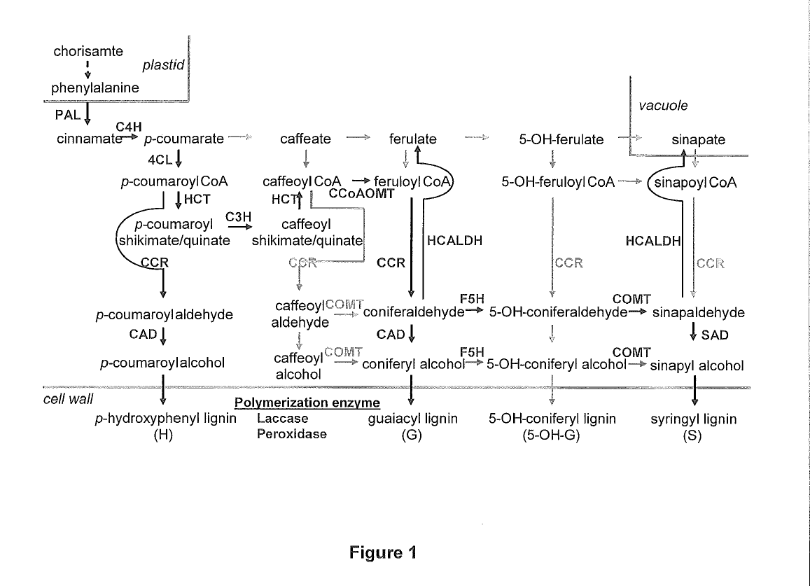

### DESCRIPTION OF PREFERRED EMBODIMENTS

Throughout this application, various publications are referenced. The disclosures of all of these publications and those references cited within those publications are hereby incorporated by reference in their entireties into this application in order to more fully describe the state of the art to which this invention pertains. The terminology used herein is for the purpose of describing specific embodiments only and is not intended to be limiting. As used herein, “a” or “an” can mean one or more, depending upon the context in which it is used. Thus, for example, reference to “a cell” can mean that at least one cell can be used. The term “about” as used herein is to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20%, preferably 10% up or down (higher or lower). The word “comprise,” “comprising,” “include,” “including,” and “includes” as used herein and in the following claims is intended to specify the presence of one or more stated features, integers, components, or steps, but they do not preclude the presence or addition of one or more other features, integers, components, steps, or groups thereof.

In one aspect, the invention provides various novel expression cassettes. In another aspect, the invention provides methods for down-regulating lignin biosynthesis of a plant or plant cell, which in turn confers an increase in digestibility and/or crude protein of the plant or part thereof as compared to a corresponding wild-type plant, wherein various expression cassettes of the invention can be used.

The term “down-regulating” or “down-regulation” as used herein means the level of expression of a nucleic acid molecule or the level of an agent such as a protein or a compound (e.g., lignin) in a plant, plant part, or plant cell is lower or reduced relative to its expression or level in a reference plant, plant part, or plant cell grown under substantially identical conditions. In the context of lignin biosynthesis, “down-regulating” or “down-regulation” as used herein means the biosynthesis of lignin in a plant, plant part, or plant cell is reduced 10% or more, for example 20% or more, preferably 30% or more, more preferably 50% or more, even more preferably 70% or more, most preferably 80% or more for example 90% relative to a plant, plant part, or plant cell lacking the expression cassettes of the invention.

The term “lignin” as used herein encompasses both lignins and lignocelluloses. More specifically, the term “lignin” refers to a heterogeneous complex of monomers and polymers in a mixture comprising p-hydroxyphenyl (H), guaiacyl (G) and syringyl (S) residues and further comprising ether linkages and carbon-carbon linkages between monomers with extensive cross-links, such as hydroxycinnamic acid (i.e. p-coumaric acid and ferulic acid) bridges, to other cell wall polymers. For example, in grass, lignin comprises monolignols para-coumaryl, coniferyl and sinapyl alcohol monomers. Lignin also refers to a polymer constructed of non-carbohydrate, alcohol units that are not fermentable, but must be separated from the cellulose and hemicellulose by chemical and other means for fermentation processes such as for producing ethanol biofuel. The production of lignin in plants has been shown to negatively correlate with cell wall digestibility of the plant (Jung et al., J. Dairy Sci., 1997, 80:1622-1628). Thus, a reduction of lignin production is likely to result in an increase in cell wall digestibility of the plants.

The term “digestibility” or “cell wall digestibility” can broadly refer to bioavailability of cell wall components either by enzymatic or chemical degradation either in vivo or in vitro. Digestibility or cell wall digestibility can also refer to the degree to which cell wall polysaccharides are hydrolyzed by microorganisms or hydrolytic enzymes into simple sugars. A plant “cell wall” is a highly heterogeneous and complex structure consisting of cellulose microfibrils embedded in a matrix of hemicellulose, pectin (only trace amounts in grasses), cell wall proteins, and phenolic compounds such as lignin. Cell wall polysaccharides must be broken down into simple sugars for digestion by livestock or for conversion into ethanol by fermenting yeast or microorganisms. Plants with increased “cell wall digestibility” have cell walls that are more digestible, whereas plants with decreased cell wall digestibility have cell walls that are less digestible.

In the field of plant biomass production, efforts have been made to, for example, manipulate the lignin biosynthesis pathway so to reduce the lignin content of plants by genetic engineering. However, it is not uncommon that the genetically engineered plants (e.g., transgenic plants) having the lignin content reduced exhibit some undesirable phenotypes such as dwarfing, slower growth, or lodging. Thus, it has been always a challenge to produce genetically engineered plants that show significant improvements in biomass characteristics while retaining growth characteristics comparable to wild type plants. Accordingly, there is still a need to develop and provide expression systems to produce transgenic plants with significant improvement in biomass characteristics, such as reduced lignin production, increased crude protein, and/or increased cell wall digestibility, while minimizing the effect of such transgene expression on phenotypic and agronomical characteristics of the plants.

### 1. Expression Cassettes of the Invention

1.1 General Concepts

The expression cassettes of the present invention generally comprise at least two components:
(a) a transcription regulating nucleotide sequence, and
(b) a nucleic acid sequence operably linked to and heterologous in relation to said transcription regulating nucleotide sequence,
wherein expression of the nucleic acid sequence manipulates the expression level of genes in the lignin biosynthesis pathway by directly reducing the expression of the genes through miRNA or indirectly regulating the expression level through the regulation of the transcription factor ZmMYB42.

As defined herein, the term “nucleic acid” and “polynucleotide” are interchangeable and refer to naturally occurring or synthetic or artificial nucleic acid or nucleotides. The term “nucleic acids” and “nucleotides” comprise DNA or RNA or any nucleotide analogue and polymers or hybrids thereof in either linear or branched, single- or double-stranded, sense or antisense form. The term also encompasses RNA/DNA hybrids. Unless otherwise indicated, a particular nucleic acid sequence also implicitly encompasses conservatively modified variants thereof such as, but not limited to, degenerate codon substitutions and complementary sequences as well as the sequence explicitly indicated. A skilled worker will recognize that DNA sequence polymorphisms, which lead to changes in the encoded amino acid sequence, may exist within a population. These genetic polymorphisms in a gene may exist between individuals within a population owing to natural variation. These natural variants usually bring about a variance of 1 to 5% in the nucleotide sequence of a particular gene. Each and every one of these nucleotide variations and resulting amino acid polymorphisms in the encoded polypeptide which are the result of natural variation and do not modify the functional activity are to be encompassed by the invention.

“Expression cassette” as used herein refers to a DNA molecule which includes sequences capable of directing expression of a particular nucleic acid sequence (e.g., which codes for a protein of interest) in an appropriate host cell, including regulatory sequences such as a promoter operably linked to a nucleic acid sequence of interest, optionally associated with transcription termination signals and/or other regulatory elements. An expression cassette may also comprise sequences required for proper translation of the nucleic acid sequence of interest. The coding region of the expression cassette usually codes for a protein of interest but may also code for a functional RNA of interest, for example, miRNA, antisense RNA, or a nontranslated RNA, in the sense or antisense direction. The expression cassette comprising the nucleic acid sequence of interest may be chimeric, meaning that at least one of its components is heterologous with respect to at least one of its other components. An expression cassette may be assembled entirely extracellularly (e.g., by recombinant cloning techniques).

The term “transcription regulating nucleotide sequence” as used herein is equivalent of the terms “promoter,” “promoter element,” or “promoter sequence” and refers to a DNA sequence which, when linked to a nucleic acid sequence of interest, is capable of controlling the transcription of the nucleic acid sequence of interest into mRNA. A transcription regulating nucleotide sequence or a promoter is typically, though not necessarily, located 5′ (i.e. upstream) of a nucleic acid sequence of interest (e.g., proximal to the transcriptional start site of a structural gene) whose transcription into mRNA it controls, and provides a site for specific binding by RNA polymerase and other transcription factors for initiation of transcription.

The term “operably linked” or “operable linkage” encompasses, for example, a sequential arrangement of the transcription regulating nucleotide sequence with the nucleic acid sequence to be expressed and, if appropriate, further regulatory elements, such as terminator or enhancers, in such a way that each of the regulatory elements can fulfill its intended function to allow, modify, facilitate or otherwise influence expression of the nucleic acid sequence under the appropriate conditions. Appropriate conditions relates to preferably the presence of the expression cassette in a plant cell. In a preferred arrangement, the nucleic acid sequence is placed down-stream (i.e. in 5′ to 3′-direction) of the transcription regulating nucleotide sequence in a way that both sequences are covalently linked. Optionally, additional sequences, such as a linker or multiple cloning sites may be inserted between the two sequences.

The term “heterologous” refers to material (nucleic acid or protein) which is obtained or derived from different source organisms, or, from different genes or proteins in the same source organism or a nucleic acid sequence to which it is not linked in nature or to which it is linked at a different location in nature. For example, transcription regulating nucleotide sequence is “heterologous to” an operably linked nucleic acid sequence when the transcription regulating nucleotide sequence is linked to, such as through ligation. As another example, a protein-coding nucleic acid sequence operably linked to a promoter, which is not the native promoter of this protein-coding sequence, is considered to be heterologous with respect to the promoter.

1.2 ZmMYB42 Expression Cassette

In one aspect, the invention provides an expression cassette for down-regulating lignin biosynthesis in a plant comprising a transcription regulating nucleotide sequence of a CCoAOMT gene operably linked to a nucleic acid sequence encoding the maize transcription factor ZmMYB42.

The maize transcription factor ZmMYB42 belongs to the R2R3-MYB subfamily of the MYB transcription factors and was shown to associate with down-regulating lignin biosynthesis in plants. A “transcription factor” as used herein refers to a protein that binds to specific DNA sequences, thereby controls the transcription of genetic information from DNA to mRNA. Transcription factors perform this function alone or with other proteins in a complex, by promoting (as an activator) or blocking (as a repressor) the recruitment of RNA polymerase to specific genes. A defining feature of transcription factors is that they contain one or more DNA binding domains (DBDs). A DNA-binding domain contains at least one motif that recognizes a specific double- or single-stranded DNA sequence or has a general affinity to DNA so to allow the transcription factor to attach to specific DNA sequences adjacent to the genes they regulate.

In one embodiment, the nucleic acid sequence encoding ZmMYB42 to be included in the expression cassette of the invention comprises a polynucleotide sequence encoding a polypeptide having the amino acid sequence of SEQ ID NO: 2 or 4, or functional variants thereof. In another embodiment, the nucleic acid sequence encoding ZmMYB42 comprises the polynucleotide sequence of SEQ ID NO: 1 or 3, or functional variants thereof.

The terms “polypeptide,” “peptide,” or “protein” are used interchangeable herein.

“Functional variants,” or “functional equivalent,” of a molecule (e.g., a polypeptide or nucleic acid sequence) is intended to mean a molecule having substantially similar sequence as compared to the non-variant molecule while retaining the activity of the non-variant molecule in whole or in part.

For nucleotide sequences comprising an open reading frame, functional variants include those sequences that, because of the degeneracy of the genetic code, encode the identical amino acid sequence of the native protein. Naturally occurring allelic variants can be identified with the use of well-known molecular biology techniques, such as, for example, with polymerase chain reaction (PCR) and hybridization techniques. Functional variant nucleotide sequences also include synthetically derived nucleotide sequences, such as those generated, for example, by using site-directed mutagenesis and for open reading frames, encode the native protein, as well as those that encode a polypeptide having amino acid substitutions relative to the native protein. A variant nucleotide sequence may also contain insertions, deletions, or substitutions of one or more nucleotides relative to the nucleotide sequence found in nature. Accordingly, a variant protein may contain insertions, deletions, or substitutions of one or more amino acid residues relative the amino acid sequence found in nature. Generally, variants of the polynucleotide sequence of SEQ ID NO: 1 or 3 or the amino acid sequence of SEQ ID NO: 2 or 4 will have at least 95%, preferably 96%, 97%, 98%, 99%, 99.1%, 99.2%, 99.3%, 99.4%, 99.5%, 99.6%, 99.7%, 99.8%, or 99.9% sequence identity to the corresponding nucleotide or amino acid sequence. The functional variants of the polynucleotide sequence of SEQ ID NO: 1 or 3 may be variants of the wild-type ZmMYB42 sequence, provided that they encode a protein retaining the activity of a MYB transcription factor in DNA binding and regulating transcription of a target gene. Likewise, the functional variants of the amino acid sequence of SEQ ID NO: 2 or 4 may be variants of the wild-type ZmMYB42 sequence, provided that they retain the activity of a MYB transcription factor in DNA binding and regulating transcription of a target gene. Moreover, in addition to the ZmMYB42 shown in SEQ ID NO: 1 or 3, which encodes the polypeptide of SEQ ID NO: 2 or 4, the skilled worker will recognize that DNA sequence polymorphisms which lead to changes in the amino acid sequences of the ZmMYB42 may exist within a population. These genetic polymorphisms in the ZmMYB42 gene may exist between individuals within a population owing to natural variation. These natural variants usually bring about a variance of 1 to 5% in the nucleotide sequence of the ZmMYB42 gene. Each and every one of these nucleotide variations and resulting amino acid polymorphisms in the ZmMYB42 which are the result of natural variation and do not modify the functional activity are to be encompassed by the invention.

As used herein, “sequence identity” or “identity” refers to a relationship between two or more polynucleotide or polypeptide sequences, as determined by aligning the sequences for maximum correspondence over a specified comparison window. As used in the art, “identity” also means the degree of sequence relatedness between polynucleotide or polypeptide sequences as determined by the match between strings of such sequences.

“Percent identity” (% identity) or “percent sequence identity” (% sequence identity) as used herein refers to the value determined by comparing two optimally aligned sequences over a specified comparison window.

Methods of sequence alignment for comparison and calculation of percent sequence identity are well known in the art. For example, the percent sequence identity may be determined with the Vector NTI Advance 10.3.0 (PC) software package (Invitrogen, 1600 Faraday Ave., Carlsbad, Calif. 92008). For percent identity calculated with Vector NTI, a gap opening penalty of 15 and a gap extension penalty of 6.66 are used for determining the percent identity of two nucleic acids. A gap opening penalty of 10 and a gap extension penalty of 0.1 are used for determining the percent identity of two polypeptides. All other parameters are set at the default settings. For purposes of a multiple alignment (e.g., Clustal W algorithm), the gap opening penalty is 10, and the gap extension penalty is 0.05 with blosum62 matrix. It is to be understood that for the purposes of determining sequence identity when comparing a DNA sequence to an RNA sequence, a thymidine nucleotide is equivalent to a uracil nucleotide. Sequence alignments and calculation of percent sequence identity may also be performed with CLUSTAL (see website at ebi.ac.uk/Tools/clustalw2/index.html), the program PileUp (Feng et al., J. Mol. Evolution., 1987, 25:351-360; Higgins et al., CABIOS, 1989, 5:151-153), or the programs Gap and BestFit (Needleman and Wunsch, J. Mol. Biol., 1970, 48:443-453; Smith and Waterman, Adv. Appl. Math., 1981, 2:482-489), which are part of the GCG software packet (Gentics Computer Group, 575 Science Drive, Madison, Wis.).

Methods of identifying homologous sequences with sequence similarity to a reference sequence are known in the art. For example, software for performing BLAST analyses for identification of homologous sequences is publicly available through the National Center for Biotechnology Information (see website at ncbi.nlm.nih.gov). PSI-BLAST (in BLAST 2.0) can also be used to perform an iterated search that detects distant relationships between molecules. When utilizing BLAST or PSI-BLAST, the default parameters of the respective programs (e.g., BLASTN for nucleotide sequences, BLASTX for proteins) can be used (see ncbi.nlm.nih.gov website). Alignment may also be performed manually by inspection. These methods may be used, for example, to identify ZmMYB42 homologs or variants and/or the corresponding coding nucleotide sequences for the use in the expression cassette of the invention.

Nucleic acid molecules encoding functional variants, homologs, analogs, and orthologs of polypeptides can be isolated. The polynucleotides encoding the respective polypeptides or primers based thereon can be used as hybridization probes according to standard hybridization techniques under stringent hybridization conditions. As used herein with regard to hybridization for DNA to a DNA blot, the term “stringent conditions” refers to hybridization overnight at 60° C. in 10×Denhart's solution, 6×SSC, 0.5% SDS, and 100 μg/ml denatured salmon sperm DNA. Blots are washed sequentially at 62° C. for 30 minutes each time in 3×SSC/0.1% SDS, followed by 1×SSC/0.1% SDS, and finally 0.1×SSC/0.1% SDS. As also used herein, in a preferred embodiment, the phrase “stringent conditions” refers to hybridization in a 6×SSC solution at 65° C. In another embodiment, “highly stringent conditions” refers to hybridization overnight at 65° C. in 10×Denhart's solution, 6×SSC, 0.5% SDS and 100 μg/ml denatured salmon sperm DNA. Blots are washed sequentially at 65° C. for 30 minutes each time in 3×SSC/0.1% SDS, followed by 1×SSC/0.1% SDS, and finally 0.1×SSC/0.1% SDS. Methods for performing nucleic acid hybridizations are well known in the art.

Accordingly, in one embodiment, the nucleic acid sequence encoding ZmMYB42 to be included in the expression cassette of the invention comprises a polynucleotide sequence selected from the group consisting of:
(a) the polynucleotide sequence of SEQ ID NO: 1,
(b) a polynucleotide sequence encoding a polypeptide having the amino acid sequence of SEQ ID NO: 2, and
(c) a polynucleotide sequence encoding a polypeptide having at least 95% identify to SEQ ID NO: 2 and having activity of a MYB transcription factor.

The polypeptide encoded by the nucleic acid sequence encoding ZmMYB42 has activity of a MYB transcription factor, preferably activity of a R2R3-MYB transcription factor, more preferably activity of ZmMYB42 transcription factor.

In another embodiment, the nucleic acid sequence encoding ZmMYB42 is operably linked to a transcription regulating nucleotide sequence of a CCoAOMT gene in the expression cassette of the invention.

The CCoAOMT (EC 2.1.1.104) converts caffeoyl CoA to feruloyl CoA and plays an essential role in the synthesis of guaiacyl lignin units as well as in the supply of substrates for the synthesis of syringyl lignin units (Guillet-Claude et al., Theor. Appl. Genet., 2004, 110:126-135). CCoAOMT genes have been isolated from various plant species, including both monocotyledonous and dicotyledonous species. For example, CCoAOMTs have been isolated from parsley (GenBank accession number Z54183), poplar (GenBank accession number AJ223621), pine (GenBank accession number AF036095), alfalfa (GenBank accession number Q40313), citrus (GenBank accession number Q9SLP8), potato (GenBank accession number Q8H₉B6), zinnia (GenBank accession number Q41720), tobacco (GenBank accession numbers Q42945 and 024151), and maize (GenBank accession numbers Q9XGD6 and Q9XGD5). Additional putative CCoAOMTs have also been identified from, for example, Arabidopsis and rice, based on the genomic sequencing data. For instance, rice CCoAOMT has been identified from the sequence of a genomic clone derived from chromosome 6 of rice (gene P0680A03.3; GenBank accession number AB023482).

The transcription regulating nucleotide sequence of a CCoAOMT gene to be included in the expression cassette of the present invention can be a CCoAOMT gene of any origin. Preferably, the transcription regulating nucleotide sequence is obtained from a CCoAOMT gene of a monocotyledonous plant, more preferably, a rice plant.

In one embodiment, the transcription regulating nucleotide sequence is from a rice CCoAMOT gene (OsCCoAOMT) comprising the polynucleotide sequence of SEQ ID NO:5 or a polynucleotide sequence encoding a polypeptide having the amino acid sequence of SEQ ID NO: 6. More preferably, the transcription regulating nucleotide sequence of a CCoAMOT gene comprises a consecutive stretch of about 25 to 1500, including 50 to 500 or 100 to 250, and up to 1,000 or 1,500, contiguous nucleotides, upstream of the OsCCoAOMT coding sequence, i.e. SEQ ID NO: 5. In a particular embodiment, the transcription regulating nucleotide sequence comprises a consecutive stretch of about 25 to 1,500, including 50 to 500 or 100 to 250, and up to 1,000 or 1,500, contiguous nucleotides having at least 50% or 60%, preferably at least 70% or 80%, more preferably at least 90%, even more preferably 95%, and most preferably at least 96%, 97%, 98%, 99% or 100% nucleic acid sequence identity with a corresponding consecutive stretch of about 25 to 1,500, including 50 to 500 or 100 to 250, and up to 1,000 or 1,500, contiguous nucleotides, upstream of the OsCCoAOMT coding sequence, i.e. SEQ ID NO: 5. The above-defined stretch of contiguous nucleotides preferably comprises one or more promoter motifs, preferably selected from the group consisting of TATA box, GC-box, CAAT-box and a transcription start site.

In another embodiment, the transcription regulating nucleotide sequence of the OsCCoAOMT gene to be included in the expression cassette of the present invention comprises the polynucleotide sequence as shown in SEQ ID NO: 7, 8, 9, or 50 or functional variants thereof. Such functional variants include, but are not limited to, fragments of SEQ ID NO: 7, 8, 9, or 50 which have substantially the same transcription regulatory activity as SEQ ID NO: 7, 8, 9, or 50, or a nucleotide sequence having at least 70% or 80%, more preferably at least 90%, even more preferably 95%, and most preferably at least 96%, 97%, 98%, 99% or 100% sequence identity to SEQ ID NO: 7, 8, 9, or 50, or a nucleotide sequence that is capable of hybridizing, preferably under stringent conditions, to the complement of SEQ ID NO: 7, 8, 9, or 50. Stringent hybridization conditions as used herein include, but not limited to, the hybridization conditions as defined above. Stringent hybridization conditions may also include hybridization conditions in 6×SSC at approximately 45° C., followed by one or more wash steps in 0.2×SSC, 0.1% SDS at 53 to 65° C., preferably at 55° C., 56° C., 57° C., 58° C., 59° C., 60° C., 61° C., 62° C., 63° C., 64° C. or 65° C. The identification and isolation of such a transcription regulating nucleotide sequence from an OsCCoAOMT gene was disclosed in more detail in US 2008/0163395, the content of which is incorporated by reference in its entirety herein. A preferred transcription regulating nucleotide sequence to be included in the expression cassette of the present invention comprises the polynucleotide sequence as shown in SEQ ID NO: 7, or variants thereof.

In yet another embodiment, transcription regulating nucleotide sequences of a OsCCoAOMT gene can not only be found upstream of the OsCCoAOMT open reading frame comprising the polynucleotide sequence of SEQ ID NO: 5, but also upstream of orthologs, paralogs or homologs of the OsCCoAOMT gene. The term “homolog(s)” is a generic term used in the art to indicate a polynucleotide or polypeptide sequence possessing a high degree of sequence relatedness to a reference sequence. Such relatedness may be quantified by determining the degree of identity and/or similarity between the two sequences. Falling within this generic term are the terms “ortholog(s)” and “paralog(s).” The term “ortholog(s)” refers to a homologous polynucleotide or polypeptide in different organisms due to ancestral relationship of these genes. The term “paralog(s)” refers to a homologous polynucleotide or polypeptide that results from one or more gene duplications within the genome of a species. The orthologs, paralogs or homologs of the OsCCoAOMT may be identified or isolated from the genome of any desired organism capable of producing lignin, preferably from another plant, according to well known techniques based on their sequence similarity to the OsCCoAOMT open reading frame having the polynucleotide sequence of SEQ ID NO: 5, e.g., hybridization, PCR or computer generated sequence comparisons. For example, all or a portion of a particular open reading frame can be used as a probe that selectively hybridizes to other gene sequences present in a population of cloned genomic DNA fragments (i.e. genomic libraries) from a chosen source organism. Further, suitable genomic libraries may be prepared from any cell or tissue of an organism. Such techniques include hybridization screening of plated DNA libraries (either plaques or colonies; see, e.g., Sambrook, 1989, Molecular Cloning: A Laboratory Manual, 2^{nd} ed., Cold Spring Harbor Laboratory, Cold Spring Harbor Laboratory Press, Cold Spring Harbor, N.Y.) and amplification by PCR using oligonucleotide primers preferably corresponding to sequence domains conserved among related polypeptide or subsequences of the nucleotide sequences provided herein. These methods are known and particularly well suited to the isolation of gene sequences from organisms closely related to the organism from which the probe sequence is derived. The application of these methods using all or a portion of the OsCCoAOMT open reading frame as probes is well suited for the isolation of gene sequences from any source organism, preferably other plant species. In a PCR approach, oligonucleotide primers can be designed for use in PCR reactions to amplify corresponding DNA sequences from cDNA or genomic DNA extracted from any plant of interest. Methods for designing PCR primers and PCR cloning are known in the art. Once a variant open reading frame is identified and isolated, the nucleic acid sequence located upstream of such a variant open reading frame can also be identified and isolated according to the methods known in the art, including, but not limited to, the genome walking technology and the thermal asymmetric interlaced polymerase chain reaction (TAIL-PCR) which can be carried out by using, e.g., commercially available kits.

Accordingly, in yet another embodiment, a variant transcription regulating nucleotide sequence to be included in the expression cassette of the invention is (i) obtained by 5′ genome walking or TAIL PCR from an open reading frame sequence as shown in SEQ ID NO: 5 or (ii) obtainable by 5′ genome walking or TAIL PCR from a open reading frame sequence being at least 80% identical to an open reading frame as shown in SEQ ID NO: 5.

Suitable oligonucleotides for use as primers in probing or amplification reactions as the PCR reaction described above, may be about 30 or fewer nucleotides in length (e.g., 9, 12, 15, 18, 20, 21, 22, 23, or 24, or any number between 9 and 30). Generally, specific primers are upwards of 14 nucleotides in length. For optimum specificity and cost effectiveness, primers of 16 to 24 nucleotides in length are preferred. Those skilled in the art are well versed in the design of primers for use in processes such as PCR. If required, probing can be done with entire restriction fragments of the gene disclosed herein which may be 100's or even 1000's of nucleotides in length.

Without significantly impairing the properties mentioned, non-essential sequences of the transcription regulating nucleotide sequence to be included in the expression cassette of the present invention can be deleted. Delimitation of the expression control sequence to particular essential regulatory regions can also be undertaken with the aid of a computer program such as the PLACE program (Higo et al., Nucleic Acids Res., 1999, 27(1):297-300; BIOBASE database “Transfac” (Biologische Datenbanken GmbH, Braunschweig)). By such measures, variant transcription regulating nucleotide sequences as specified above can be artificially generated. Moreover, processes for mutagenizing nucleic acid sequences are known to the skilled worker and include, e.g., the use of oligonucleotides having one or more mutations compared with the region to be mutated (e.g., within the framework of a site-specific mutagenesis). Primers having approximately 15 to approximately 75 nucleotides or more are typically employed, with preferably about 10 to about 25 or more nucleotide residues being located on both sides of a sequence to be modified. Details and procedure for said mutagenesis processes are familiar to the skilled worker (Kunkel et al., Methods Enzymol., 1987, 154:367-382; Tomic et al., Nucl. Acids Res., 1990, 12:1656; Upender et al., Biotechniques, 1995, 18(1):29-30; U.S. Pat. No. 4,237,224). A mutagenesis can also be achieved by treatment of, for example, vectors comprising the transcription regulating nucleotide sequence to be included in the expression cassette of the invention with mutagenizing agents such as hydroxylamine.

The transcription regulating nucleotide sequence of a CCoAOMT gene to be included in the expression cassettes of the invention is capable of directing expression of the operably linked nucleic acid sequence encoding ZmMYB42 in a plant in such a way that the production of lignin in the transgenic plant is reduced due to the overexpression of ZmMYB42, which results in an increase in digestibility of the plant. For example, when ZmMYB42 was overexpressed under the control of a transcription regulating nucleotide sequence comprising the polynucleotide sequence of SEQ ID NO: 7 and a terminator isolated from the rice CCoAOMT gene (e.g., SEQ ID NO: 10), the ZmMYB42 was constitutively expressed in the transgenic plants to reduce lignin content and increase digestibility. Transgenic events produced agronomical characteristics comparable to the corresponding wild type plants; no major negative influences due to weeds, disease or insects, and no major biomass yield loss were observed. Preferably, the transcription regulating nucleotide sequence to be included in the expression cassette of the invention has substantially the same transcription regulating activity as the transcription regulating nucleotide sequence described by SEQ ID NO: 7, 8, 9, or 50.

The transcription regulating activity of a specific transcription regulating nucleotide sequence is considered substantially the same or equivalent if transcription is initiated preferentially or specifically in essentially the same tissue(s) during essentially the same developmental stage(s) than the original promoter (e.g., in leaf or constitutive in all or most tissues) under otherwise identical conditions, i.e. in combination with the set of additional regulatory elements (e.g., introns, transcription terminator sequences, and/or 5′-untranslated regions) and the same nucleic acid sequence to be expressed in the same plant expression system. Such expression profile may be demonstrated using reporter genes operably linked to the transcription regulating sequence. Preferred reporter genes in this context include, but not limited to, green fluorescence protein, chloramphenicol transferase, luciferase, β-glucuronidase or β-galactosidase (as described further below). Especially preferred is β-glucuronidase. The skilled worker is familiar with such reporter genes.

With respect to transcription regulating nucleotide sequences with constitutive expression activity (e.g., constitutive promoters), the term “at most times” means a transcription regulating activity (as demonstrated by an β-glucuronidase assay as known in the art) preferably during at least 50%, preferably at least 70%, more preferably at least 90% of the development cycle of a plant comprising the respective expression cassette stably integrated into its chromosomal DNA. Similarly, the term “in most tissues” means a transcription regulating activity (as demonstrated by an β-glucuronidase assay as known in the art) in tissues which together account to preferably at least 50%, preferably at least 70%, more preferably at least 90% of the entire biomass of the a plant comprising the respective expression cassette stably integrated into its chromosomal DNA.

In the context of lignin biosynthesis modification by regulating the expression of ZmMYB42, the activity of a specific transcription regulating nucleotide sequence is considered substantially the same or equivalent to the original promoter if such a transcription regulating nucleotide sequence is capable of driving the expression of ZmMYB42 in a plant so to produce a reduction in lignin biosynthesis and an increase in digestibility of the plant.

1.3 pZmCOMT Expression Cassette

In another aspect, the invention provides a novel transcription regulating nucleotide sequence isolated from the transcription regulatory region of the maize COMT gene, which differs from known maize COMT transcription regulating nucleotide sequences, and an expression cassette comprising such a novel transcription regulating nucleotide sequence.

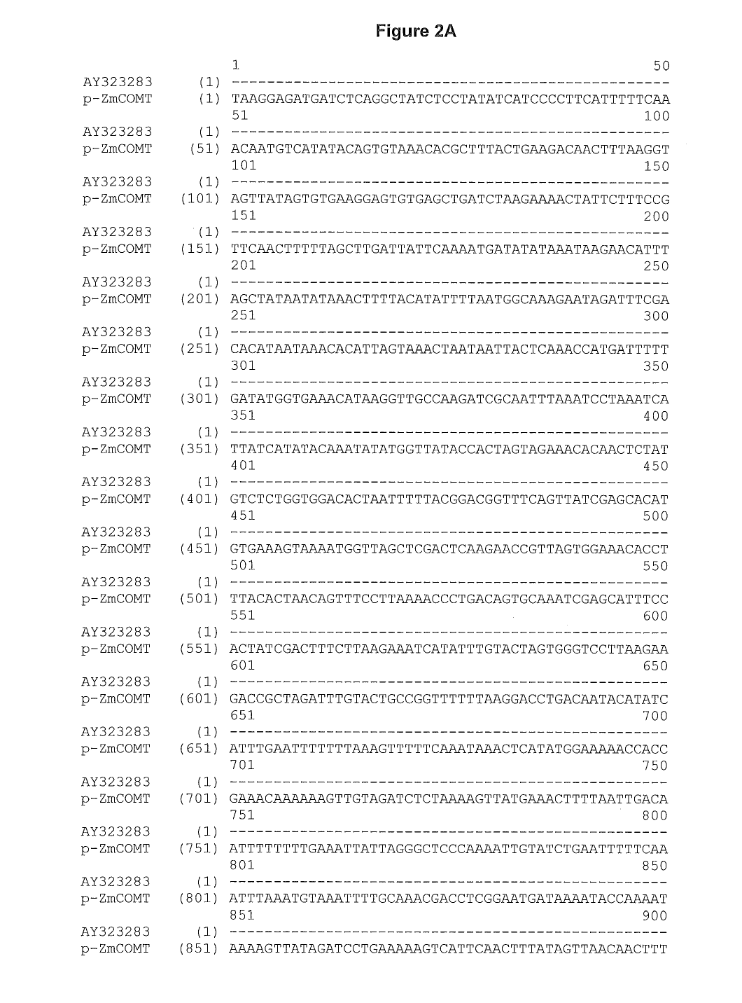

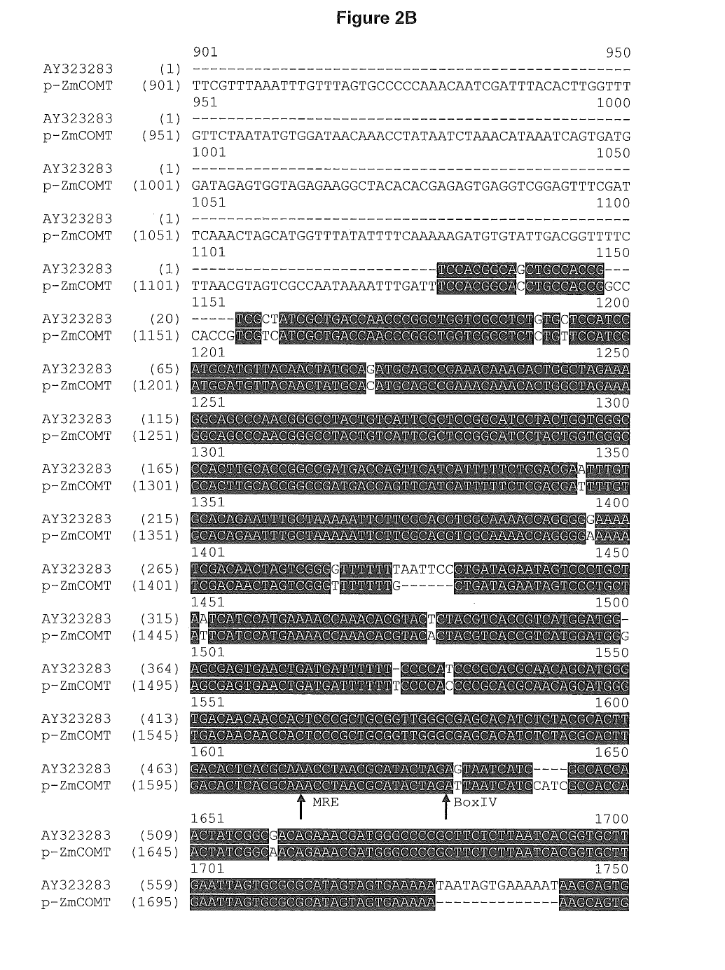

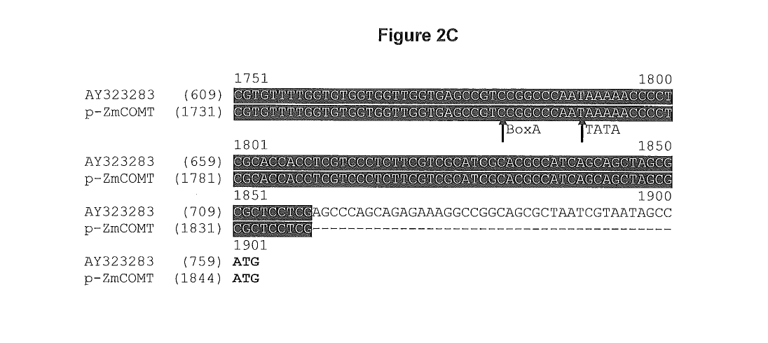

The novel ZmCOMT promoter described herein differs from known ZmCOMT promoters in several aspects. A sequence alignment between the ZmCOMT promoter of the invention and the ZmCOMT promoter sequence previously isolated from the inbred line W64A (GenBank Accession No. AY323283; Guillet-Claude et al., Theor. Appl. Genet., 2004, 110:126-135) is provided in FIG. 2. As shown in FIG. 2, in addition to various mismatches and gaps, these two ZmCOMT promoter sequences differ significantly at both their 5′ and 3′ ends. At the 5′ end, the ZmCOMT promoter of the invention is 1128 nucleotides longer than the ZmCOMT promoter sequence as shown in GenBank Accession No. AY323283. At the 3′ end, the promoter sequence of AY323283 contains 41 extra nucleotides immediately upstream of the start codon ATG. A further sequence comparison shows that this extra 41-nucleotide region located at the 3′ end of the promoter region of AY323283 is also presented in the ZmCOMT promoter sequence of the originally-reported ZmCOMT gene (M73235; Collazo et al., Plant Mol. Biol., 1992, 20:857-867).

Moreover, the novel ZmCOMT promoter possesses different transcription regulating activity compared to that of known ZmCOMT promoters. For instance, a known ZmCOMT promoter was reported to be active in all tissues undergoing lignification such as the small vascular strands of young leaves and the vascular bundles of stems (Capellades, Plant Mol. Biol., 1996, 31:307-322). However, the ZmCOMT promoter described herein does not drive GUS expression in tissues where the promoter activity was detected by Capellades et al.

Accordingly, in one embodiment, the invention provides a transcription regulating nucleotide sequence comprising the polynucleotide sequence of SEQ ID NO: 13, or variants thereof.

The term “variants” with respect to SEQ ID NO: 13 has the same meaning as “functional variants” or “functional equivalents” previously defined. Specifically, with respect to a transcription regulating nucleotide sequence, the term “variants” is intended to encompass nucleic acids with substantially similar sequences as compared to the non-variant molecule as well as nucleic acids that are fragments of the non-variant molecule, in which the transcription regulatory activity of the non-variant molecule is retained in whole or in part. Examples of variants include, but not limited to, naturally occurring allelic variants such as those can be identified using well-known molecular biology techniques and synthetically derived nucleotide sequences such as those generated, for example, by using site-directed mutagenesis. In the context of the novel ZmCOMT promoter as described herein, the variants of the polynucleotide sequence of SEQ ID NO: 13 will have at least 750 nucleotides, preferably 1,000 or 1,200 nucleotides, more preferably 1,200 or 1,400 nucleotides, even more preferably 1,600 or 1,800 nucleotides, having at least 90%, preferably 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the polynucleotide sequence of SEQ ID NO: 13, and capable of driving expression of a heterologous nucleic acid sequence that is operably linked thereto. The variants of the polynucleotide sequence of SEQ ID NO: 13 also encompass fragments of at least 750 nucleotides, preferably 1,000 or 1,200 nucleotides, more preferably 1,200 or 1,400 nucleotides, even more preferably 1,600 or 1,800 nucleotides, of the polynucleotide sequence of SEQ ID NO: 13, and capable of driving expression of a heterologous nucleic acid sequence that is operably linked thereto.

Methods for identifying and/or isolating transcription regulating nucleotide sequence variants of the invention are known in the art. Similarly, methods for testing and determining whether a candidate polynucleotide sequence possesses the prerequisite transcription regulatory activity (e.g., tissue-specific or developmental regulated) are known in the art. Examples of suitable methods include, but not limited to, the methods described in the other sections herein such as Section 1.2 above. Preferably, the transcription regulating nucleotide sequence variants so identified are capable of directing expression of a heterologous nucleic acid sequence that is operably linked thereto, more preferably, in a manner that is substantially the same as that of the transcription regulating nucleotide sequence of SEQ ID NO: 13. “Substantially the same” as used in this context means that the isolated transcription regulating nucleotide sequence is capable of initiating expression of an operably linked nucleic acid sequence preferentially or specifically in essentially the same tissue during essentially the same developmental stage(s) as the transcription regulating nucleotide sequence of SEQ ID NO: 13 under otherwise identical conditions (i.e. using comparable additional regulatory elements in a comparable plant expression system).

The transcription regulating nucleotide sequences of the invention may be employed for numerous expression purposes such as, for example, expression of a protein, or expression of an antisense RNA, sense or double-stranded RNA. Preferably, expression of the nucleic acid sequence confers to the plant an agronomically valuable trait, which in a preferred embodiment is decreased content of lignin in a plant and/or increased cell wall digestibility of the plant.

“Agronomically valuable trait” as used herein refers to any phenotype in a plant organism that is useful or advantageous for food production or food products, including plant parts and plant products. Non-food agricultural products such as paper, etc. are also included. A partial list of agronomically valuable traits includes improvement of pest resistance (e.g., Melchers et al., Curr. Opin. Plant Biol., 2000, 3(2):147-52), vigor, development time (time to harvest), enhanced nutrient content, novel growth patterns, flavors or colors, salt, heat, drought and cold tolerance (e.g., Sakamoto et al., J. Exp. Bot., 2000, 51(342):81-8; Saijo et al., Plant J., 2000, 23(3):319-327; Yeo et al., Mol. Cells, 2000, 10(3):263-8; Cushman et al., Curr. Opin. Plant Biol., 2000, 3(2):117-24), and the like. Preferably, agronomically valuable traits do not include selectable marker genes (e.g., genes encoding herbicide or antibiotic resistance used only to facilitate detection or selection of transformed cells), hormone biosynthesis genes leading to the production of a plant hormone (e.g., auxins, gibberllins, cytokinins, abscisic acid and ethylene that are used only for selection), or reporter genes (e.g., luciferase, glucuronidase, chloramphenicol acetyl transferase (CAT), etc.). Those of skill will recognize that there are numerous polynucleotides from which to choose to confer these and other agronomically valuable traits.

Accordingly, in one embodiment, the invention further provides an expression cassette comprising the novel transcription regulating nucleotide sequence of the maize COMT gene as described herein operably linked to a heterologous nucleic acid sequence of interest.

The term “nucleic acid sequence of interest” refers to a nucleic acid sequence which shall be expressed under the control of the transcription regulating nucleotide sequence of the invention. The nucleic acid sequence of interest may be obtained from, for example, a disease resistance gene such as a bacterial disease resistance gene, a fungal disease resistance gene, or a viral disease resistance gene, a gene affecting grain composition or quality, or a nutrient utilization gene. Other examples of genes from which the nucleic acid sequence of interest may be obtained include, but not limited to, an insect resistance gene, a herbicide resistance gene, a mycotoxin reduction gene, a male sterility gene, a selectable or screenable marker gene, a gene affecting plant agronomic characteristics such as yield, standability, digestibility, and the like, or an environment or stress resistance or tolerance gene. Preferably, the nucleic acid sequence of interest encodes a polypeptide which negatively affects the lignin biosynthesis of a plant when overexpressed and thus, increases digestibility of the plant. A non-limiting example of such a nucleic acid sequence of interest may be a nucleic acid sequence encoding the maize MYB42 transcription factor as described in Section 1.2. It is to be understood that nucleic acid sequences encoding polypeptides that down-regulate the lignin biosynthesis pathway of a plant when overexpressed can also be used as nucleic acid sequences of interest in the expression cassette of the invention and therefore, are within the scope of the invention.

The nucleic acid sequence of interest may also encode a biologically active RNA molecule that affects expression of an endogenous gene. An “endogenous gene” refers to a native gene in its natural location in the genome of an organism. A “biologically active RNA molecule” includes, but not limited to, antisense RNAs, sense or double-stranded RNAs, ribozymes, miRNAs, or siRNAs. The underlying biological principles of action of the aforementioned biologically active RNA molecules are well known in the art. Moreover, the person skilled in the art is well aware of how to obtain nucleic acids which encode such biologically active RNA molecules. Preferably, the production of a biologically active RNA molecule down-regulates expression of the endogenous gene via the posttranscriptional gene silencing (or “PTGS”) mechanism, including but not limited to, RNA interference (“RNAi”).

The term “posttranscriptional gene silencing” or “PTGS” refers to silencing of gene expression in plants after transcription. PTGS may be gene specific or nongene specific, such that a group of related genes are silenced.

“RNA interference” or “RNAi” as used herein refers to the silencing of a gene wherein the translation of a gene is down regulating or decreasing of gene expression by RNAi molecules (e.g., miRNAs). It is the process of sequence-specific, post-transcriptional gene silencing in animals and plants, initiated by RNAi molecules that are homologous in their duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit completely or partially the function of a target RNA.

As used herein, the term “antisense” refers to a nucleotide sequence that is inverted relative to its normal orientation for transcription and so expresses an RNA transcript that is complementary to a target gene mRNA molecule expressed within the host cell (e.g., it can hybridize to the target gene mRNA molecule through Watson-Crick base pairing). An antisense strand may be constructed in a number of different ways, provided that it is capable of interfering with the expression of a target gene. For example, the antisense strand can be constructed by inverting the coding region (or a portion thereof) of the target gene relative to its normal orientation for transcription to allow the transcription of its complement (e.g., RNAs encoded by the antisense and sense gene may be complementary). Furthermore, the antisense oligonucleotide strand need not have the same intron or exon pattern as the target gene, and non-coding segments of the target gene may be equally effective in achieving antisense suppression of target gene expression as coding segments. In the context of gene silencing, the term “antisense” is understood to mean a nucleic acid having a sequence complementary to a target sequence, for example a messenger RNA (mRNA) sequence, the blocking of whose expression is sought to be initiated by hybridization with the target sequence.

The term “double-stranded RNA” molecule or “dsRNA” molecule as used herein comprises a sense RNA fragment of a nucleotide sequence and an antisense RNA fragment of the nucleotide sequence, which both comprise nucleotide sequences complementary to one another, thereby allowing the sense and antisense RNA fragments to pair and form a double-stranded RNA molecule. Preferably the terms refer to a double-stranded RNA molecule capable, when introduced into a cell or organism, of at least partially reducing the level of an mRNA species present in a cell or a cell of an organism.

The term “microRNA molecules” or “miRNAs” refers to small, noncoding RNA molecules of 18-25 nucleotides that function through RNA-mediated interference (RNAi) and related pathways to regulate the expression of target genes. MicroRNAs (or miRNAs) have been found in a diverse array of eukaryotes, including mammals and plants. miRNAs arise from larger precursor molecules, i.e. pre-miRNAs, which share a characteristic secondary structure comprising stem-loop structures, also described as hairpin structures, wherein the stems may comprise bulges of non complementary base pairs or base pairs inserted in a stem on only one side of the stem. Genetic and biochemical studies have indicated that miRNAs are processed to their mature forms by Dicer, an RNAse III family nuclease, and function through RNA-mediated interference (RNAi) and related pathways to regulate the expression of target genes (Hannon, Nature, 2002, 418:244-251; Pasquinelli et al., Annu. Rev. Cell Dev. Biol., 2002, 18:495-513; Bartel D. P., Cell, 2004, 116:281-297). miRNAs may be configured or engineered to permit experimental manipulation of gene expression in plant cells as synthetic silencing triggers, such as artificial miRNAs (“amiRNAs,” Ossowski et al., Plant J., 2008, 53:674-690). Silencing by amiRNAs involves the RNAi machinery and correlates with the production of small interfering RNAs (siRNAs), which are a signature of RNAi.

In the context of modifying lignin biosynthesis of a plant for increasing the digestibility of the plant, the endogenous gene, the expression of which is to be down-regulated by the biologically active RNA molecule, is preferably a gene which encodes a polypeptide having enzymatic activity in the lignin biosynthesis pathway. Examples of such an endogenous gene include, but not limited to, genes encoding phenylalanine ammonia lyase (PAL), cinnamate 4-hydrolase (C4H), 4-coumarate:CoA ligase (4CL), hydroxycinnamoyl-CoA:shikimate and quinate hydroxyl-cinnamoyl transferase (HCT), p-coumarate 3-hydroxylase (C3H), caffeoyl-CoA 3-O-methyltransferase (CCoAOMT), cinnamoyl-CoA reductase (CCR), cinnamyl alcohol dehydrogenase (CAD), sinapyl alcohol dehydrogenase (SAD), ferulate 5-hydroxylase (F5H), caffeic acid O-methyltransferase (COMT), hydroxycinnamaldehyde dehydrogenase (HCALDH), peroxidase, or laccase. In one embodiment, the endogenous gene to be down-regulated by the biologically active RNA molecule is a COMT or CCoAOMT gene, preferably a maize COMT gene. In another embodiment, the biologically active RNA molecule is, for example, a miRNA as described in Section 1.4 below. It is also within the scope of the invention that an expression cassette as described herein may comprise a transcription regulating nucleotide sequence of the maize COMT gene as provided herein operably linked to a miR166 precursor sequence as described in more detail in Section 1.4 below.

1.4 MicroRNA Expression Cassette

In yet another aspect, the present invention provides an expression cassette which comprises a miR166 precursor sequence that is engineered in such a way to produce a miRNA sequence which reduces expression of a gene in the lignin biosynthesis pathway.

In the process of producing a mature miRNA, a miRNA precursor preferentially produces one silencing RNA (sRNA) duplex, the miRNA-miRNA star duplex. It is found that, when both miRNA and miRNA star (miRNA*) sequences are altered without changing structural features such as mismatch or bulges, it often leads to high-level accumulation of an miRNA with a sequence that is unrelated to the miRNA normally produced by the precursor. Artificial miRNAs (amiRNAs, also called synthetic miRNAs) were therefore generated and used both in human cell lines (Zeng et al., Mol. Cell, 2002, 9:1327-1333) and in plants (e.g., Parizotto et al., Genes Dev., 2004, 18:2237-2242; Alvarez et al., Plant Cell, 2006, 18:1134-1151; Schwab et al., Plant Cell, 2006, 18:1121-1133).

The mature 19-25 nt miRNA sequence that is loaded into the RISC(RNA-induced silencing complex) binds to its target mRNA. Before it is loaded into the RISC, miRNA is bound to the miRNA* sequence by complementary base pairing with 2 bp 3′ overhangs and in that form is called the miRNA/miRNA* duplex. The miRNA/miRNA* duplex is processed out of the pre-miRNA hairpin fold-back by Dicer.

Properties important for efficient small RNA-mediated gene silencing, particularly in the use of miRNAs include, for example, the allowable degree of mismatches (0 to 5, corresponding to about 75% identity or more) between the miRNA sequence and its target sequence as well as their locations, were identified and built into Web-based tools for automating amiRNA design. A non-limiting example of such Web-based tools is Web MicroRNA Designer (“WMD”) as described in Ossowski et al. (Plant J., 2008, 53:674-690).

As used herein, the term “miR166 precursor sequence” refers to the maize miR166 precursor sequence comprising the polynucleotide sequence of SEQ ID NO: 16, and functional variants thereof. The term “functional variant(s),” “functional equivalent(s),” or “variant(s)” as used herein is meant to have the same meaning as defined above. In the context of miRNA precursors, specifically the miR166 precursor sequence, functional variants can have at least 70, 75% to 80%, preferably 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the polynucleotide sequence of the maize miR166 precursor sequence as shown in SEQ ID NO: 16, and are capable of folding into proper secondary structure that can be recognized and processed by the plant cell machinery to release the mature miRNA molecule. Functional variants of the maize miR166 precursor sequence may also be a functional fragment of at least 100 or 150 nucleotides, preferably 175 or 250 nucleotides, more preferably 300 or 400 nucleotides, even more preferably 500, 600, or 693 nucleotides, of the polynucleotide sequence of SEQ ID NO: 16, and are capable of folding into proper secondary structure that can be recognized and processed by the plant cell machinery to release the mature miRNA molecule. Non-limiting examples of variants of the miR166 precursor are shown as SEQ ID NO: 47, SEQ ID NO: 48, or SEQ ID NO: 49. A functional variant of the miR166 precursor can comprise, for example, SEQ ID NO: 47, SEQ ID NO: 48, or SEQ ID NO: 49, or a sequence having at least 70, 75% to 80%, preferably 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as shown in SEQ ID NO: 47, SEQ ID NO: 48, or SEQ ID NO: 49.

The miR166 precursor sequence as described above is engineered in such a way to produce a miRNA sequence which reduces expression of a gene in the lignin biosynthesis pathway. The native maize miR166 precursor sequence, such as that provided in SEQ ID NO: 16, contains the native miR166 sequence having the polynucleotide sequence of SEQ ID NO: 18 located at positions 89 to 109 of SEQ ID NO: 16, with the corresponding miR166* sequence having the polynucleotide sequence of SEQ ID NO: 17 located at positions 32 to 52 of SEQ ID NO: 16. By replacing the native miR166 sequence and the corresponding miR166* sequence contained in the miR166 precursor sequence with miRNA sequences designed for targeting a gene in the lignin biosynthesis pathway, this engineered miR166 precursor sequence will be processed by the plant cell machinery resulting in a miRNA sequence that targets the gene in the lignin biosynthesis pathway and reduces expression of said gene. The replacement of the native miR166 and the corresponding miR166* sequences with miRNA and miRNA* sequences of interest need not be at the exact nucleotide positions where the native sequences are found in the miR166 precursor sequence and need not be the exact same length of sequences, so long as the miRNA sequence of interest is properly processed from the engineered miR166 precursor to a level sufficient to reduce expression of the targeted gene. The miR166 precursor sequence may be engineered by replacing a first segment of about 19-24 contiguous nucleotides located between positions corresponding to about nucleotide 32 and nucleotide 55 of SEQ ID NO: 16 with a first nucleotide sequence of about 19-24 nucleotides, and a second segment of about 19-24 contiguous nucleotides located between positions corresponding to about nucleotide 86 and nucleotide 109 of SEQ ID NO: 16 with a second nucleotide sequence of about 19-24 nucleotides. Preferably, the first nucleotide sequence is the same length as the first segment and the second nucleotide sequence is the same length as the second segment. In general, the segments to be replaced or the polynucleotide sequences used for replacement have a length of 21 nucleotides, but they can also have a length of 19 to 24 nucleotides, such as 19, 20, 21, 22, 23, or 24 nucleotides. However, smaller variations in length can be tolerated. Thus, the polynucleotide sequence replacing the segment can be, for example, one, two, or three nucleotides longer or shorter than the segment.

The replacement can be done by various techniques of cloning known to the person skilled in the art and as described, for example, in Maniatis et al., Molecular Cloning: A Laboratory Manual, 2nd Ed., 1989, Cold Spring Harbor Laboratory, Cold Spring Harbor, N.Y.; Silhavy et al., Experiments with Gene Fusions, 1984, Cold Spring Harbor Laboratory, Cold Spring Harbor, N.Y.; Ausubel et al., Current Protocols in Molecular Biology, 1987, Greene Publishing Assoc. and Wiley Interscience; Gelvin et al., Plant Molecular Biology Manual, 1990, Kluwer Academic Publisher, Dordrecht, The Netherlands. Preferably, the replacement is done by a PCR-mediated mutation approach.

Complete complementarity between the miRNA and miRNA* sequences or between the miRNA and the target mRNA sequence is not required to achieve efficient miRNA processing and/or efficient gene silencing or attenuation.

The term “complementary” or “complementarity” as used herein refers to two nucleotide sequences which comprise anti-parallel nucleotide sequences capable of pairing with one another by the base-pairing rules. For example, the sequence 5′-AGT-3′ is complementary to the sequence 5′-ACT-3′. Complementarity can be “partial” or “total.” “Partial” complementarity means that one or more nucleotide bases is mismatched between two nucleic acids. “Total” or “complete” complementarity means each and every nucleotide base is matched between two nucleic acids according to the base pairing rules. Accordingly, the term “substantially complementary” as used herein refers to a first nucleotide sequence (e.g., miRNA sequence) is at least 70 to 75%, preferably at least 80%, more preferably at least 85% or 90%, even more preferably at least 91%, 92%, 93%, 94% or 95%, still more preferably at least 96%, 97%, 98%, or 99% complementary to a second nucleotide sequence (e.g., miRNA* sequence). Substantially complementary also refers to, preferably over its length (e.g., of 19 to 25 nucleotides), a first nucleotide sequence (e.g., miRNA sequence) having not more than 10, preferably not more than 5 or 8, more preferably not more than 3 or 4, even more preferably not more than 2, most preferably not more than 1 mismatch in comparison to the complete complement to a second nucleotide sequence (e.g., miRNA* sequence). Sequence comparisons may be carried out using programs and methods as described previously, for example, in Section 1.2. In the context of miRNA engineering, mismatches can be more tolerable in the 3′ portion of the miRNA sequence than the 5′ portion and central sequences. Thus, in some embodiments, the pairing between the miRNA and miRNA* sequences, or the miRNA sequence and the target mRNA sequence, can be exact pairing at the 5′ portion and central sequences of about positions 2-12 of the miRNA sequence and not more than 10, preferably not more than 5 or 8, more preferably not more than 3 or 4, even more preferably not more than 2, most preferably not more than 1 mismatch in the 3′ portion of the miRNA sequence. In the context of miRNA processing, the miRNA sequence is preferably substantially complementary to the miRNA* sequence so that a mature miRNA sequence can be properly processed and released from the miRNA precursor. In the context of gene silencing using miRNA, the miRNA sequence is preferably substantially complementary to the target mRNA sequence so that the expression of the target gene can be reduced.

For RNA interference to occur, the miRNA sequence produced by the engineered miR166 precursor sequence of the invention needs to form a RNA-RNA duplex with a portion of the mRNA transcribed from a gene of interest to trigger cleavage and degradation of the target mRNA in order to reduce the expression of the gene of interest. The miRNA sequence may target any portion of the mRNA, including, but not limited to, 5′-untranslated region (5′-UTR), 3′-untranslated region (3′-UTR), or any part of the coding region. In one embodiment, the miR166 precursor sequence is engineered in such a way to produce a miRNA sequence which reduces expression of a gene in the lignin biosynthesis pathway. Examples of such genes include, but not limited to, a gene encoding phenylalanine ammonia lyase (PAL), cinnamate 4-hydrolase (C4H), 4-coumarate:CoA ligase (4CL), hydroxycinnamoyl-CoA:shikimate and quinate hydroxyl-cinnamoyl transferase (HCT), p-coumarate 3-hydroxylase (C3H), caffeoyl-CoA 3-O-methyltransferase (CCoAOMT), cinnamoyl-CoA reductase (CCR), cinnamyl alcohol dehydrogenase (CAD), sinapyl alcohol dehydrogenase (SAD), ferulate 5-hydroxylase (F5H), caffeic acid O-methyltransferase (COMT), hydroxycinnamaldehyde dehydrogenase (HCALDH), peroxidase, or laccase.

In another embodiment, the miR166 precursor sequence of the invention is engineered in a way to produce a miRNA sequence that reduces expression of a COMT gene, preferably a maize COMT gene. The miRNA sequences may be designed to target either the 5′-UTR, the 3′-UTR, or any part of the coding region of the mRNA transcribed from the COMT gene, preferably the maize COMT gene. An example of the coding sequence of the maize COMT gene, including the 5′- and 3′-UTR sequences, is shown in SEQ ID NO: 11. Based on this COMT coding sequence, non-limiting examples of miRNA sequences may be designed to comprise the polynucleotide sequence as shown in SEQ ID NO: 20 or 22, with the corresponding miRNA* sequence as shown in SEQ ID NO: 19 and 21, respectively. The miRNA may also comprise the polynucleotide sequence as shown in SEQ ID NO: 45 or 46. Accordingly, the engineered miR166 precursor sequence included in the expression cassette of the invention may comprise, but not limited to, the polynucleotide sequence as shown in SEQ ID NO: 23, 24, or 25.

As some mismatches between a miRNA sequence and its mRNA target sequence are generally tolerable, it is also within the scope of the invention that miRNA sequences that reduce expression of a maize COMT gene comprise a polynucleotide sequence having at least 70%, preferably at least 80%, more preferably at least 85% or 90%, even more preferably at least 95%, 97%, 98% or 99%, most preferably 100%, sequence identity to the polynucleotide sequence as shown in SEQ ID NO: 20 or 22. Once the miRNA sequence is determined, a miRNA* sequence variations can be designed. Preferably, the corresponding miRNA* sequence comprises a polynucleotide sequence having at least 70%, preferably at least 80%, more preferably at least 85% or 90%, even more preferably at least 95%, 97%, 98% or 99%, most preferably 100%, sequence identity to the polynucleotide sequence as shown in SEQ ID NO: 19 or 21. In one embodiment, the variants of SEQ ID NO: 20 or 22 remain substantially complementary to the corresponding variants of SEQ ID NO: 19 or 21, respectively. In another embodiment, the variants of SEQ ID NO: 20 or 22 retain the ability to reduce expression of a maize COMT gene.

For expressing the engineered miR166 precursor sequence in plants, the miR166 precursor sequence need be operably linked to an appropriate transcription regulating nucleotide sequence, or promoter, preferably a plant-specific promoter. The term “plant-specific promoter” means principally any promoter which is capable of driving the expression of a nucleic acid operably linked thereto, in particular foreign nucleic acid sequences or genes, in plants or plant parts, plant cells, plant tissues, plant cultures. In this context, the expression specificity of said plant-specific promoter can be for example constitutive, inducible, developmentally regulated, tissue-specific or tissue-preferential, organ-specific or organ-preferential, cell type-specific or cell type-preferential, spatial-specific or spatial-preferential, and/or temporal-specific or temporal-preferential. Suitable promoters useful for the expression cassettes of the invention include any promoter that is capable of initiating transcription in a plant in a specific or preferential cell type or tissue at a specific or preferential developmental stage.

Such promoters include, but not limited to, those that can be obtained from plants, plant viruses and bacteria that contain genes that are expressed in plants, such as Agrobacterium and Rhizobium.

Constitutive promoters are generally active under most environmental conditions and states of development or cell differentiation. Useful constitutive promoters for plants include those obtained from Ti- or Ri-plasmids, from plant cells, plant viruses or other organisms whose promoters are found to be functional in plants. Bacterial promoters that function in plants, and thus are suitable for use in the present invention include, but not limited to, the octopine synthetase promoter, the nopaline synthase promoter, and the mannopine synthetase promoter from the T-DNA of Agrobacterium. Likewise, viral promoters that function in plants can also be used in the present invention. Examples of viral promoters include, but not limited to, the promoter isolated from sugarcane bacilliform virus (SvBV; U.S. Pat. No. 6,489,462; Nadiya et al., Biotechnology, 2010, published online), the cauliflower mosaic virus (CaMV) 35S transcription initiation region (Franck et al., Cell, 1980, 21:285-294; Odell et al., Nature, 1985, 313:810-812; Shewmaker et al., Virology, 1985, 140:281-288; Gardner et al., Plant Mol. Biol., 1986, 6:221-228), the cauliflower mosaic virus (CaMV) 19S transcription initiation region (U.S. Pat. No. 5,352,605 and WO 84/02913) and region VI promoters, and the full-length transcript promoter from Figwort mosaic virus. Other suitable constitutive promoters for use in plants include, but not limited to, actin promoters such as the rice actin promoter (McElroy et al., Plant Cell, 1990, 2:163-171) or the Arabidopsis actin promoter, histone promoters, tubulin promoters, or the mannopine synthase promoter (MAS), ubiquitin or poly-ubiquitin promoters (Sun and Callis, Plant J., 1997, 11(5):1017-1027; Cristensen et al., Plant Mol. Biol., 1992, 18:675-689; Christensen et al., Plant Mol. Biol., 1989 12:619-632; Bruce et al., Proc. Natl. Acad. Sci. USA, 1989, 86:9692-9696; Holtorf et al., Plant Mol. Biol., 1995, 29:637-649), the Mac or DoubleMac promoters (U.S. Pat. No. 5,106,739; Comai et al., Plant Mol. Biol., 1990, 15:373-381), Rubisco small subunit (SSU) promoter (U.S. Pat. No. 4,962,028), the legumin B promoter (GenBank Acc. No. X03677), the TR dual promoter, the Smas promoter (Velten et al., EMBO J., 1984, 3:2723-2730), the cinnamyl alcohol dehydrogenase promoter (U.S. Pat. No. 5,683,439), the promoters of the vacuolar ATPase subunits, the pEMU promoter (Last et al., Theor. Appl. Genet., 1991, 81:581-588), the maize H3 histone promoter (Lepetit et al., Mol. Gen. Genet., 1992, 231:276-285; Atanassova et al., Plant J., 1992, 2(3):291-300), β-conglycinin promoter, the phaseolin promoter, the ADH promoter, and heat-shock promoters, the nitrilase promoter from Arabidopsis thaliana (WO 03/008596; GenBank Acc. No. U38846, nucleotides 3,862 to 5,325 or else 5,342), promoter of a proline-rich protein from wheat (WO 91/13991), the promoter of the Pisum sativum ptxA gene, and other promoters active in plant cells that are known to those of skill in the art.

Inducible promoters are active under certain environmental conditions, such as the presence or absence of a nutrient or metabolite, heat or cold, light, pathogen attack, anaerobic conditions, and the like. A promoter is inducible, if its activity, measured on the amount of RNA produced under control of the promoter, is at least 30%, 40%, 50%, preferably at least 60%, 70%, 80%, 90%, more preferably at least 100%, 200%, 300%, higher in its induced state than in its un-induced state. An inducible promoter can be induced in response to a chemical (for review, see Gatz et al., Annu. Rev. Plant Physiol. Plant Mol. Biol., 1997, 48:89-108), environmental or physical stimulus, or may be “stress-inducible,” i.e. activated when a plant is exposed to various stress conditions, or “pathogen-inducible,” i.e. activated when a plant is exposed to exposure to various pathogens. Chemically inducible promoters are especially suitable if gene expression is desired in a time specific manner. Examples for such promoters include, but not limited to, a salicylic acid-inducible promoter (WO 95/19443), a benzene-sulfonamide-inducible promoter (EP 0 388 186), a tetracycline-inducible promoter (Gatz et al., Mol. Gen. Genetics, 1991, 227:229-237), an abscisic acid-inducible promoter (EP 0 335 528), an ethanol-cyclohexanone-inducible promoter (WO 93/21334), and the promoter of the glutathione-S transferase isoform II gene (GST-1′-27) (WO 93/01294). A promoter that responds to an inducing agent to which plants do not normally respond can also be utilized, such as the glucocorticosteroid hormone inducible promoter from a steroid hormone gene (Schena et al., Proc. Natl. Acad. Sci. USA, 1991, 88:10421). Promoters responding to biotic or abiotic stress conditions are also suitable inducible promoters. Examples of such promoters include, but not limited to, the pathogen-inducible promoter of the PRP1 gene (Ward et al., Plant Mol. Biol., 1993, 22:361-366), the heat-inducible hsp70 or hsp80 promoter from tomato (U.S. Pat. No. 5,187,267), the chill-inducible α-amylase promoter from potato (WO 96/12814), the light-inducible PPDK promoter, or the wound-induced pinII promoter (EP 375091).

A cell-specific, tissue-specific, or organ-specific promoter is one that is capable of preferentially initiating transcription in certain types of cells, tissues, or organs, such as leaves, stems, roots, flowers, fruits, anthers, ovaries, pollen, seed tissue, green tissue, or meristem. A promoter is cell-, tissue- or organ-specific, if its activity, measured on the amount of RNA produced under control of the promoter, is at least 30%, 40%, 50%, preferably at least 60%, 70%, 80%, 90%, more preferably at least 100%, 200%, 300%, higher in a particular cell-type, tissue or organ, then in other cell-types or tissues of the same plant, preferably the other cell-types or tissues are cell types or tissues of the same plant organ, e.g., leaves or roots. In the case of organ specific promoters, the promoter activity has to be compared to the promoter activity in other plant organs, e.g., leaves, stems, flowers or seeds. For example, the tissue-specific ES promoter from tomato is particularly useful for directing expression in fruits (see, e.g., Lincoln et al., Proc. Natl. Acad. Sci. USA, 1988, 84:2793-2797; Deikman et al., EMBO J., 1988, 7:3315-3320; Deikman et al., Plant Physiol., 1992, 100:2013-2017). Seed-specific or seed-preferential promoters are preferentially expressed during seed development and/or germination, which can be embryo-, endosperm-, and/or seed coat-specific or preferential. See Thompson et al., BioEs-says, 1989, 10:108. Examples of seed-specific or preferential promoters include, but not limited to, those derived from MAC1 gene from maize (Sheridan et al., Genetics, 1996, 142:1009-1020), Cat3 gene from maize (GenBank Accession No. L05934), the gene encoding oleosin 18 kD from maize (GenBank Accession No. J05212), viviparous-1 gene from Arabidopsis (Genbank Accession No. U93215), the gene encoding oleosin from Arabidopsis (Genbank Accession No. Z17657), Atmycl gene from Arabidopsis (Urao et al., Plant Mol. Biol., 1996, 32:571-576), the 2S seed storage protein gene family from Arabidopsis (Conceicao et al., Plant J., 1994, 5:493-505), the gene encoding oleosin 20 kD from Brassica napus (GenBank Accession No. M63985), napin gene from Brassica napus (GenBank Accession No. J02798; Joseffson et al., J. Biol. Chem., 1987, 262:12196-12201), the napin gene family (e.g., from Brassica napus; Sjodahl et al., Planta, 1995, 197:264-271, U.S. Pat. No. 5,608,152; Stalberg et al., Planta, 1996, 199:515-519), the gene encoding the 2S storage protein from Brassica napus (Dasgupta et al., Gene, 1993, 133:301-302), the genes encoding oleosin A (Genbank Accession No. U09118) and oleosin B (Genbank Accession No. U09119) from soybean, the gene encoding low molecular weight sulphur rich protein from soybean (Choi et al., Mol. Gen. Genet., 1995, 246:266-268), the phaseolin gene (U.S. Pat. No. 5,504,200; Bustos et al., Plant Cell, 1989, 1(9):839-853; Murai et al., Science, 1983, 23:476-482; Sengupta-Gopalan et al., Proc. Natl, Acad. Sci. USA, 1985, 82:3320-3324), the 2S albumin gene, the legumin gene (Shirsat et al., Mol. Gen. Genet., 1989, 215(2):326-331), the USP (unknown seed protein) gene, the sucrose binding protein gene (WO 00/26388), the legumin B4 gene (LeB4; Fiedler et al., Biotechnology, 1995, 13(10):1090-1093; Baumlein et al., Plant J., 1992, 2(2):233-239; Baumlein et al., Mol. Gen. Genet., 1991, 225(3):459-467; Baumlein et al., Mol. Gen. Genet., 1991, 225:121-128), the Arabidopsis oleosin gene (WO 98/45461), the Brassica Bce4 gene (WO 91/13980), genes encoding the “high-molecular-weight glutenin” (HMWG), gliadin, branching enzyme, ADP-glucose pyrophosphatase (AGPase) or starch synthase.

Other suitable tissue- or organ-specific or preferential promoters include a leaf-specific and light-induced promoter such as that from cab or Rubisco (Timko et al., Nature, 1985, 318:579-582; Simpson et al., EMBO J., 1985, 4:2723-2729), an anther-specific promoter such as that from LAT52 (Twell et al., Mol. Gen. Genet., 1989, 217:240-245), a pollen-specific promoter such as that from ZmI3 (Guerrero et al., Mol. Gen. Genet., 1993, 224:161-168), and a microspore-preferred promoter such as that from apg (Twell et al., Sex. Plant Reprod., 1983, 6:217-224). Also suitable promoters are, for example, specific promoters for tubers, storage roots or roots such as, for example, the class I patatin promoter (B33), the potato cathepsin D inhibitor promoter, the starch synthase (GBSS1) promoter or the sporamin promoter, and fruit-specific promoters such as, for example, the tomato fruit-specific promoter (EP 0409625). Promoters which are furthermore suitable are those which ensure leaf-specific or leaf-preferential expression. Further examples of promoters which may be mentioned are the potato cytosolic FBPase promoter (WO 98/18940), the Rubisco (ribulose-1,5-bisphosphate carboxylase) SSU (small subunit) promoter or the potato ST-LSI promoter (Stockhaus of al., EMBO J., 1989, 8(9):2445-2451). Other suitable promoters are those which govern expression in seeds and plant embryos. Further suitable promoters are, for example, fruit-maturation-specific promoters such as, for example, the tomato fruit-maturation-specific promoter (WO 94/21794), flower-specific promoters such as, for example, the phytoene synthase promoter (WO 92/16635) or the promoter of the P1-rr gene (WO 98/22593) or another node-specific promoter as described in EP 0249676 may be used advantageously. The promoter may also be a pith-specific promoter, such as the promoter isolated from a plant TrpA gene as described in WO 93/07278.

Developmentally regulated or developmental stage-preferred promoters are preferentially expressed at certain stages of development. Suitable developmental regulated promoters include, but not limited to, fruit-maturation-specific promoters, such as, for example, the fruit-maturation-specific promoter from tomato (WO 94/21794, EP 0409625). Developmental regulated promoters also include partly the tissue-specific promoters described above since individual tissues are, naturally, formed as a function of the development. An example of a development-regulated promoter is described in Baerson et al. (Plant Mol. Biol., 1993, 22(2):255-267).

Other promoters or promoter elements suitable for the expression cassettes of the invention include, but not limited to, promoters or promoter elements capable of modifying the expression-governing characteristics. Thus, for example, the tissue-specific expression may take place in addition as a function of certain stress factors, owing to genetic control sequences. Such elements are, for example, described for water stress, abscisic acid (Lam and Chua, J. Biol. Chem., 1991, 266(26):17131-17135) and heat stress (Schoffl et al., Molecular & General Genetics, 1989, 217(2-3):246-253).

In one embodiment, the transcription regulating nucleotide sequence or promoter used to direct the expression of the miR166 precursor sequence in the expression cassette of the invention is the transcription regulating nucleotide sequence obtained from a CCoAOMT gene, preferably a rice CCoAOMT gene, as described above in Section 1.2. Equally preferred is the transcription regulating nucleotide sequence of a COMT gene, preferably a maize COMT gene, as described above in Section 1.3.

In another embodiment, the aforementioned expression cassette may further comprise a second miR166 precursor sequence that is operably linked to the transcription regulating nucleotide sequence and the first miR166 precursor sequence included in the expression cassette so that the same transcription regulating nucleotide sequence directs the expression of both first and second miR166 precursor sequences. Preferably, the second miR166 precursor sequence has the characteristics as described above. The second miR166 precursor sequence may be engineered in such a way to produce a second miRNA sequence that is either the same or different from the miRNA sequence produced by the first miR166 precursor sequence. If the second miRNA sequence is a different miRNA sequence, this second miRNA sequence may target a different portion of the same mRNA sequence transcribed from the same gene of interest or it may target a portion of a mRNA sequence transcribed from a different gene of interest. In one of the preferred embodiments, at least one of the miR166 precursor sequences is engineered in a way to produce a miRNA sequence that reduces expression of a gene in the lignin biosynthesis pathway as disclosed above. In a further embodiment, the miRNA is designed to reduce expression of a COMT gene, more preferably a maize COMT gene. Non-limiting examples of such miR166 precursor sequences may comprise the polynucleotide sequence of SEQ ID NO: 23, 24, or 25. Without unduly limiting the scope of the invention, an example of such an expression cassette may comprise the polynucleotide sequence as described in SEQ ID NO: 26, which is generated by combining the miR166 precursor sequences of SEQ ID NO: 24 and 25 in tandem. Functional equivalents of such an expression cassette are within the scope of the present invention.

### 2. Other Regulatory Elements

The aforementioned expression cassettes may further comprise other regulatory elements. The term “regulatory elements” encompasses all sequences which may influence construction or function of the expression cassette. Regulatory elements may, for example, modify transcription and/or translation in prokaryotic or eukaryotic organism. Thus, the expression profile of the nucleic acid sequence included in the aforementioned expression cassettes (e.g., ZmMYB42 coding sequence or the miR166 precursor sequence) may be modulated depending on the combination of the transcription regulating nucleotide sequence and the other regulatory element(s) comprised in the expression cassette.

In one embodiment, the aforementioned expression cassettes may further comprise at least one additional regulatory element selected from the group consisting of:
(a) 5′-untranslated regions (or 5′-UTR),
(b) intron sequences, and
(c) transcription termination sequences (or terminators).

A variety of 5′ and 3′ transcriptional regulatory sequences are available for use in the expression cassettes of the present invention. As the DNA sequence between the transcription initiation site and the start codon of the coding sequence, i.e., the 5′-untranslated sequence, can influence gene expression, one may wish to include a particular 5′-untranslated sequence in the expression cassettes of the invention. Preferred 5′-untranslated sequences include those sequences predicted to direct optimum expression of the attached gene, i.e., consensus 5′-untranslated sequences which may increase or maintain mRNA stability and prevent inappropriate initiation of translation. The choice of such sequences will be known to those of skill in the art. Sequences that are obtained from genes that are highly expressed in plants will be most preferred. Also preferred is the 5′-untranslated region obtained from the same gene as the transcription regulating sequence to be included in the expression cassette of the invention.

Additionally, it is known in the art that a number of non-translated leader sequences are capable of enhancing expression, for example, leader sequences derived from viruses. For example, leader sequences from Tobacco Mosaic Virus (TMV), Maize Chlorotic Mottle Virus (MCMV), and Alfalfa Mosaic Virus (AMV) have been shown to be effective in enhancing expression (e.g., Gallie 1987; Skuzeski 1990). Other viral leader sequences known in the art include, but not limited to, Picornavirus leaders, for example, EMCV leader (Encephalomyocarditis 5′ noncoding region) (Elroy-Stein 1989), Potyvirus leaders, for example, TEV leader (Tobacco Etch Virus), MDMV leader (Maize Dwarf Mosaic Virus), Human immunoglobulin heavy-chain binding protein (BiP) leader (Macejak 1991), and untranslated leader from the coat protein mRNA of alfalfa mosaic virus (AMV RNA 4) (Jobling 1987).

The 3′ regulatory sequence preferably includes from about 50 to about 1,000, more preferably about 100 to about 1,000, base pairs and contains plant transcriptional and translational termination sequences. Transcription termination sequences, or terminators, are responsible for the termination of transcription and correct mRNA polyadenylation. Thus, the terminators preferably comprise a sequence inducing polyadenylation. The terminator may be heterologous with respect to the transcription regulating nucleotide sequence and/or the nucleic acid sequence to be expressed, but may also be the natural terminator of the gene from which the transcription regulating nucleotide sequence and/or the nucleic acid sequence to be expressed is obtained. In one embodiment, the terminator is heterologous to the transcription regulating nucleotide sequence and/or the nucleic acid sequence to be expressed. In another embodiment, the terminator is the natural terminator of the gene of the transcription regulating nucleotide sequence.

Appropriate terminators and those which are known to function in plants include, but not limited to, CaMV 35S terminator, the tml terminator, the nopaline synthase (NOS) terminator, the pea rbcS E9 terminator, the terminator for the T7 transcript from the octopine synthase gene of Agrobacterium tumefaciens, and the 3′ end of the protease inhibitor I or II genes from potato or tomato. Alternatively, one also could use a gamma coixin, oleosin 3 or other terminator from the genus Coix. Preferred 3′ regulatory elements include, but not limited to, those from the nopaline synthase (NOS) gene of Agrobacterium tumefaciens (Bevan 1983), the terminator for the T7 transcript from the octopine synthase gene of Agrobacterium tumefaciens, and the 3′ end of the protease inhibitor I or II genes from potato or tomato.

In one embodiment, where the transcription regulating nucleotide sequence is obtained from a CCoAOMT gene as described above, preferably from a rice CCoAOMT gene, preferably the terminator is the terminator obtained from a CCoAOMT gene, either the same CCoAOMT gene from which the transcription regulating nucleotide sequence is obtained or a different CCoAOMT gene. In another embodiment, the transcription regulating nucleotide sequence and the terminator are obtained from the same CCoAOMT gene, preferably, both from the rice CCoAOMT gene. A non-limiting example of a terminator to be included in the expression cassettes of the invention comprises the polynucleotide sequence as described by SEQ ID NO: 10.

Transcription regulatory elements can also include intron sequences that have been shown to enhance gene expression in transgenic plants, particularly in monocotyledonous plants. The intron sequence is preferably inserted in the aforementioned expression cassettes in the 5′-untranslated region of the nucleic acid sequence to be expressed (i.e. between the transcription regulating nucleotide sequence and the nucleic acid sequence to be expressed). Preferably, such expression enhancing intron sequences are from monocotyledonous plants. Preferred intron sequences include, but not limited to, intron sequences from Adh1 (Callis 1987), bronzel, actin1, actin 2 (WO 00/760067), or the sucrose synthase intron (Vasil 1989). See The Maize Handbook, Chapter 116 (Freeling and Walbot, Eds., Springer, New York, 1994). More preferably, the intron sequences are:
(a) the introns of the Zea mays ubiquitin gene, preferably intron I thereof, most preferably the intron sequence as described by SEQ ID NO: 30,
(b) the introns of the rice actin gene, preferably intron I thereof, most preferably the intron sequence as described by nucleotide 121 to 568 of the sequence described by GenBank Accession No. X63830,
(c) the introns of the Zea mays alcohol dehydrogenase (adh) gene, preferably intron 6 thereof, most preferably the intron sequence as described by nucleotide 3,135 to 3,476 of the sequence described by GenBank Accession No. X04049, and
(d) the introns of rice Metallothionin1 gene, preferably intron I thereof, most preferably the intron sequence as described by SEQ ID NO: 31.

Additional intron sequences with expression enhancing properties in plants may also be identified and isolated according to the disclosure of US 2006/0094976 (hereby incorporated by reference in its entirety).

### 3. Recombinant Constructs and Vectors

The aforementioned expression cassettes are preferably comprised in an recombinant construct and/or a vector, preferably a plant transformation vector. Numerous vectors for recombinant DNA manipulation or plant transformation are known to the person skilled in the pertinent art. The selection of vector will depend upon the host cell employed. Similarly, the selection of plant transformation vector will depend upon the preferred transformation technique and the target species for transformation.

3.1 Recombinant Constructs

Another aspect of the invention refers to a recombinant construct comprising at least one of the aforementioned expression cassettes. Preferably, the recombinant construct comprises at least one aforementioned expression cassette comprising other regulatory elements described herein for directing the expression of the nucleic acid sequence comprised in the aforementioned expression cassette in an appropriate host cell. More preferably, the recombinant construct comprises at least one aforementioned expression cassette with at least one terminator. Optionally, or in another embodiment, the recombinant construct may comprise at least one aforementioned expression cassette further comprising at least one expression enhancing sequence such as an intron sequence as exemplified herein, for example, in Section 2.

It is further within the scope of the invention that a recombinant construct may comprise more than one aforementioned expression cassette. For example, a recombinant construct may comprise at least one ZmMYB42 expression cassette as described in Section 1.2 above in combination with at least one pZmCOMT expression cassette as described in Section 1.3 or at least one miRNA expression cassette as described in Section 1.4 above. Other non-limiting examples of suitable recombinant construct may comprise at least one pZmCOMT expression cassette as described in Section 1.3 and at least one miRNA expression cassette as described in Section 1.4 above, at least two different ZmMYB42 expression cassettes as described in Section 1.2, at least two different pZmCOMT expression cassettes as described in Section 1.3, or at least two different miRNA expression cassettes as described in Section 1.4 above. “Different ZmMYB42 expression cassette,” “different pZmCOMT expression cassette,” or “different miRNA expression cassette” as used herein is to mean an expression cassette comprising the principal components of the particular expression cassette as described in Sections 1.2, 1.3, or 1.4, respectively, but may comprise different transcription regulating nucleotide sequence and/or different nucleic acid sequence to be expressed. It is also to be understood that each expression cassette to be included in the recombinant construct may further comprise at least one regulatory element of the same or different type as described herein.

3.2 Vectors

Another aspect of the invention refers to a vector comprising the aforementioned expression cassette or a recombinant construct derived therefrom. The term “vector,” preferably, encompasses phage, plasmid, viral or retroviral vectors as well as artificial chromosomes, such as bacterial or yeast artificial chromosomes. Moreover, the term also relates to targeting constructs which allow for random or site-directed integration of the targeting construct into genomic DNA. Such target constructs, preferably, comprise DNA of sufficient length for either homologous or heterologous recombination. The vector encompassing the expression cassettes or recombinant constructs of the invention, preferably, further comprises selectable markers as described below for propagation and/or selection in a host. The vector may be incorporated into a host cell by various techniques well known in the art. If introduced into a host cell, the vector may reside in the cytoplasm or may be incorporated into the genome. In the latter case, it is to be understood that the vector may further comprise nucleic acid sequences which allow for homologous recombination or heterologous insertion.

Vectors can be introduced into prokaryotic or eukaryotic cells via conventional transformation or transfection techniques. The terms “transformation” and “transfection,” conjugation and transduction, as used in the present context, are intended to comprise a multiplicity of processes known in the art for introducing foreign nucleic acid (e.g., DNA) into a host cell, including, but not limited to, calcium phosphate, rubidium chloride or calcium chloride co-precipitation, DEAE-dextran-mediated transfection, lipofection, natural competence, carbon-based clusters, chemically mediated transfer, electroporation or particle bombardment (e.g., “gene-gun”). Suitable methods for the transformation or transfection of host cells, including plant cells, can be found in Sambrook et al. (Molecular Cloning: A Laboratory Manual, 2^{nd} ed., 1989, Cold Spring Harbor Laboratory, Cold Spring Harbor Laboratory Press, Cold Spring Harbor, N.Y.) and other laboratory manuals, such as Methods in Molecular Biology (Gartland and Davey eds., 1995, Vol. 44, Agrobacterium Protocols, Humana Press, Totowa, N.J.). Alternatively, a plasmid vector may be introduced by heat shock or electroporation techniques. Should the vector be a virus, it may be packaged in vitro using an appropriate packaging cell line prior to application to host cells. Retroviral vectors may be replication competent or replication defective. In the latter case, viral propagation generally will occur only in complementing host or host cells. Preferably, the vector referred to herein is suitable as a cloning vector, i.e. replicable in microbial systems. Such vectors ensure efficient cloning in bacteria and, preferably, yeasts or fungi and make possible the stable transformation of plants. Examples of suitable vectors include, but not limited to, various binary and co-integrated vector systems which are suitable for the T-DNA-mediated transformation as described herein. These vector systems, preferably, also comprise further cis-regulatory elements as described herein, such as selection markers or reporter genes.

3.3 Vector Elements

Recombinant constructs and the vectors derived therefrom may comprise further functional elements. The term “functional element” is to be understood in the broad sense and means all those elements which have an effect on the generation, multiplication or function of the recombinant constructs, vectors or transgenic organisms according to the invention. Examples of such function elements include, but not limited to, selection marker genes, reporter genes, origins of replication, elements necessary for Agrobacterium-mediated transformation, and multiple cloning sites (MCS).

Selection marker genes are useful to select and separate successfully transformed cells. Preferably, within the method of the invention one marker may be employed for selection in a prokaryotic host, while another marker may be employed for selection in a eukaryotic host, particularly the plant species host. The marker may confer resistance against a biocide, such as antibiotics, toxins, heavy metals, or the like, or may function by complementation, imparting prototrophy to an auxotrophic host. Preferred selection marker genes for plants may include, but not limited to, negative selection markers, positive selection markers, and counter selection markers.

Negative selection markers include markers which confer a resistance to a biocidal compound such as a metabolic inhibitor (e.g., 2-deoxyglucose-6-phosphate, WO 98/45456), antibiotics (e.g., kanamycin, G 418, bleomycin or hygromycin) or herbicides (e.g., phosphinothricin or glyphosate). Especially preferred negative selection markers are those which confer resistance to herbicides. These markers can be used, beside their function as a selection marker, to confer a herbicide resistance trait to the resulting transgenic plant. Examples of negative selection markers include, but not limited to:
Phosphinothricin acetyltransferases (PAT; also named Bialophos resistance; bar; de Block et al., EMBO J., 1987, 6:2513-2518; EP 0333033; U.S. Pat. No. 4,975,374);
5-enolpyruvylshikimate-3-phosphate synthase (EPSPS; U.S. Pat. No. 5,633,435) or glyphosate oxidoreductase gene (U.S. Pat. No. 5,463,175) conferring resistance to Glyphosate (N-phosphonomethyl glycine) (Shah et al., Science, 1986, 233:478);
Glyphosate degrading enzymes (Glyphosate oxidoreductase; gox);
Dalapon inactivating dehalogenases (deh);
Sulfonylurea- and imidazolinone-inactivating acetolactate synthases (for example mutated ALS variants with, for example, the S4 and/or Hra mutation);
Bromoxynil degrading nitrilases (bxn);
Kanamycin- or G418-resistance genes (NPTII or NPTI) coding for neomycin phosphotransferases (Fraley et al., Proc. Natl. Acad. Sci. USA, 1983, 80:4803), which expresses an enzyme conferring resistance to the antibiotic kanamycin and the related antibiotics neomycin, paromomycin, gentamicin, and G418;
2-Deoxyglucose-6-phosphate phosphatase (DOGR1—Gene product; WO 98/45456; EP 0807836) conferring resistance against 2-desoxyglucose (Randez-Gil et al., Yeast, 1995, 11:1233-1240);
Hygromycin phosphotransferase (HPT), which mediates resistance to hygromycin (Vanden Elzen et al., Plant Mol. Biol., 1985, 5:299); and
Dihydrofolate reductase (Eichholtz et al., Somatic Cell and Molecular Genetics, 1987, 13:67-76).

Additional negative selection marker genes of bacterial origin that confer resistance to antibiotics include the aadA gene, which confers resistance to the antibiotic spectinomycin, gentamycin acetyl transferase, streptomycin phosphotransferase (SPT), aminoglycoside-3-adenyl transferase and the bleomycin resistance determinant (Svab et al., Plant Mol. Biol., 1990, 14:197; Jones et al., Mol. Gen. Genet., 1987, 210:86; Hille et al., Plant Mol. Biol., 1986, 7:171; Hayford et al., Plant Physiol., 1988, 86:1216). Other negative selection markers include those confer resistance against the toxic effects imposed by D-amino acids like e.g., D-alanine and D-serine (WO 03/060133; Erikson et al., Nat Biotechnol., 2004, 22(4):455-458), the daol gene encoding a D-amino acid oxidase (EC 1.4.3.3; GenBank Accession No. U60066) from Rhodotorula gracilis (Rhodosporidium toruloides), and the dsdA gene encoding a D-serine deaminase (EC 4.3.1.18; GenBank Accession No. J01603) from E. coli. Depending on the employed D-amino acid, the D-amino acid oxidase markers can be employed as dual function marker offering negative selection (e.g., when combined with for example D-alanine or D-serine) or counter selection (e.g., when combined with D-leucine or D-isoleucine).

Positive selection markers include markers which confer a growth advantage to a transformed plant in comparison with a non-transformed one. Genes like isopentenyltransferase from Agrobacterium tumefaciens (strain PO22; Genbank Accession No. AB025109) may, as a key enzyme of the cytokinin biosynthesis, facilitate regeneration of transformed plants (e.g., by selection on cytokinin-free medium). Corresponding selection methods are described in Ebinuma et al. (Proc. Natl. Acad. Sci. USA, 2000, 94:2117-2121) and Ebinuma et al. (“Selection of marker-free transgenic plants using the oncogenes (ipt, rol A, B, C) of Agrobacterium as selectable markers,” 2000, in Molecular Biology of Woody Plants, Kluwer Academic Publishers). Additional positive selection markers, which confer a growth advantage to a transformed plant in comparison with a non-transformed one, are described in, for example, EP 0601092. Growth stimulation selection markers may include, but not limited to, β-glucuronidase (in combination with, for example, cytokinin glucuronide), mannose-6-phosphate isomerase (in combination with mannose), UDP-galactose-4-epimerase (in combination with, for example, galactose), wherein mannose-6-phosphate isomerase in combination with mannose is especially preferred.

Counter selection markers are especially suitable to select organisms with defined deleted sequences comprising said marker (Koprek et al., Plant J., 1999, 19(6):719-726). Examples for counter selection marker include, but not limited to, thymidine kinases (TK), cytosine deaminases (Gleave et al., Plant Mol. Biol., 1999, 40(2):223-35; Perera et al., Plant Mol. Biol., 1993, 23(4):793-799; Stougaard, Plant J., 1993, 3:755-761), cytochrom P450 proteins (Koprek et al., Plant J., 1999, 19(6):719-726), haloalkan dehalogenases (Naested, Plant J., 1999, 18:571-576), iaaH gene products (Sundaresan et al., Gene Develop., 1995, 9:1797-1810), cytosine deaminase codA (Schlaman and Hooykaas, Plant J., 1997, 11:1377-1385), and tms2 gene products (Fedoroff and Smith, Plant J., 1993, 3:273-289).

Reporter genes encode readily quantifiable proteins and, via their color or enzyme activity, make possible an assessment of the transformation efficacy, the site of expression or the time of expression. Very especially preferred in this context are genes encoding reporter proteins (Schenborn and Groskreutz, Mol. Biotechnol., 1999, 13(1):29-44) such as the green fluorescent protein (GFP) (Haseloff et al., Proc. Natl. Acad. Sci, USA, 1997, 94(6):2122-2127; Sheen et al., Plant J., 1995, 8(5):777-784; Reichel et al., Proc. Natl. Acad. Sci. USA, 1996, 93(12):5888-5893; Chui et al., Curr. Biol., 1996, 6:325-330; Leffel et al., Biotechniques, 1997, 23(5):912-918; Tian et al., Plant Cell Rep., 1997, 16:267-271; WO 97/41228), chloramphenicol transferase, a luciferase (Millar et al., Plant Mol. Biol. Rep., 1992, 10:324-414; Ow et al., Science, 1986, 234:856-859), the aequorin gene (Prasher et al., Biochem. Biophys. Res. Commun., 1985, 126(3):1259-1268), β-galactosidase, R locus gene (encoding a protein which regulates the production of anthocyanin pigments (red coloring) in plant tissue and thus makes possible the direct analysis of the promoter activity without addition of further auxiliary substances or chromogenic substrates; see Dellaporta et al., 1988, In: Chromosome Structure and Function: Impact of New Concepts, 18th Stadler Genetics Symposium, 11:263-282; Ludwig et al., Science, 1990, 247:449), with β-glucuronidase (GUS) being very especially preferred (Jefferson, Plant Mol. Bio. Rep., 1987, 5:387-405; Jefferson et al., EMBO J., 1987, 6:3901-3907). β-glucuronidase (GUS) expression is detected by a blue color on incubation of the tissue with 5-bromo-4-chloro-3-indolyl-β-D-glucuronic acid, bacterial luciferase (LUX) expression is detected by light emission, firefly luciferase (LUC) expression is detected by light emission after incubation with luciferin, and galactosidase expression is detected by a bright blue color after the tissue was stained with 5-bromo-4-chloro-3-indolyl-β-D-galactopyranoside. Reporter genes may also be used as scorable markers as alternatives to antibiotic resistance markers. Such markers can be used to detect the presence or to measure the level of expression of the transferred gene. The use of scorable markers in plants to identify or tag genetically modified cells works well when efficiency of modification of the cell is high. Origins of replication which ensure amplification of the recombinant constructs or vectors according to the invention in, for example, E. coli. Examples of suitable origins of replication include, but not limited to, OR1 (origin of DNA replication), the pBR322 on or the P15A on (Sambrook et al., Molecular Cloning: A Laboratory Manual, 2^{nd} ed., Cold Spring Harbor Laboratory, Cold Spring Harbor Laboratory Press, Cold Spring Harbor, N.Y., 1989). Additional examples for replication systems functional in E. coli, are ColE1, pSC101, pACYC184, or the like. In addition to or in place of the E. coli replication system, a broad host range replication system may be employed, such as the replication systems of the P-1 Incompatibility plasmids, e.g., pRK290. These plasmids are particularly effective with armed and disarmed Ti-plasmids for transfer of T-DNA to the plant host.

Other functional elements may be included in the recombinant constructs and the vector derived therefrom of the invention include, but not limited to, other genetic control elements for excision of the inserted sequences from the genome, elements necessary for Agrobacterium-mediated transformation, and multiple cloning sites (MCS).

Other genetic control elements for excision permit removal of the inserted sequences from the genome. Methods based on the cre/lox (Dale and Ow, Proc. Natl. Acad. Sci. USA, 1991, 88:10558-10562; Sauer, Methods, 1998, 14(4):381-392; Odell et al., Mol. Gen. Genet., 1990, 223:369-378), FLP/FRT (Lysnik et al., Nucleic Acid Research, 1993, 21:969-975), or Ac/Ds system (Lawson et al., Mol. Gen. Genet., 1994, 245:608-615; Wader et al., in Tomato Technology (Alan R. Liss, Inc.), 1987, pp. 189-198; U.S. Pat. No. 5,225,341; Baker et al., EMBO J., 1987, 6:1547-1554) permit removal of a specific DNA sequence from the genome of the host organism, if appropriate, in a tissue-specific and/or inducible manner. In this context, the control sequences may mean the specific flanking sequences (e.g., lox sequences) which later allow removal (e.g., by means of cre recombinase) of a specific DNA sequence.

Elements necessary for Agrobacterium-mediated transformation may include, but not limited to, the right and/or, optionally, left border of the T-DNA or the vir region.

Multiple cloning sites (MCS) can be included in the recombinant construct or the vector of the invention to enable and facilitate the insertion of one or more nucleic acid sequences.

3.4 Vectors for Plant Transformation

If Agrobacteria are used for plant transformation, the recombinant construct is to be integrated into specific plasmid vectors, either into a shuttle or intermediate vector, or into a binary vector. If a Ti or Ri plasmid is to be used for the transformation, at least the right border, but in most cases the right and the left border, of the Ti or Ri plasmid T-DNA is flanking the region with the recombinant construct to be introduced into the plant genome. Preferably, binary vectors for the Agrobacterium transformation can be used. Binary vectors are capable of replicating both in E. coli and in Agrobacterium. They preferably comprise a selection marker gene and a linker or polylinker flanked by the right and, optionally, left T-DNA border sequence. They can be transformed directly into Agrobacterium (Holsters et al., Mol. Gen. Genet., 1978, 163:181-187). A selection marker gene may be included in the vector which permits a selection of transformed Agrobacteria (e.g., the nptIIl gene). The Agrobacterium, which acts as host organism in this case, may already comprise a disarmed (i.e. non-oncogenic) plasmid with the vir region for transferring the T-DNA to the plant cell. The use of T-DNA for the transformation of plant cells has been studied and described extensively (e.g., EP 0120516; Hoekema, In: The Binary Plant Vector System, Offsetdrukkerij Kanters B.V., Alblasserdam, Chapter V; An et al., EMBO J., 1985, 4:277-287). A variety of binary vectors are known and available for transformation using Agrobacterium, such as, for example, pB1101.2 or pBIN19 (Clontech Laboratories, Inc. USA; Bevan et al., Nucl. Acids Res., 1984, 12:8711), pBinAR, pPZP200 or pPTV.

Transformation can also be realized without the use of Agrobacterium. Non-Agrobacterium transformation circumvents the requirement for T-DNA sequences in the chosen transformation vector and consequently vectors lacking these sequences can be utilized in addition to vectors such as the ones described above which contain T-DNA sequences. Transformation techniques that do not rely on Agrobacterium include, but not limited to, transformation via particle bombardment, protoplast uptake (e.g., PEG and electroporation) and microinjection, all are well known in the art. The choice of vector depends largely on the preferred selection for the species being transformed. Typical vectors suitable for non-Agrobacterium transformation include pCIB3064, pSOG19, and pSOG35 (see e.g., U.S. Pat. No. 5,639,949).

4. Introduction of Expression Cassette into Cells and Organisms

The aforementioned expression cassettes, or the recombinant constructs or vectors derived therefrom, can be introduced into a cell or an organism in various ways known to the skilled worker. “To introduce” is to be understood in the broad sense and comprises, for example, all those methods suitable for directly or indirectly introducing a DNA or RNA molecule into an organism or a cell, compartment, tissue, organ or seed of same, or generating it therein. The introduction can bring about either a transient presence or a stable presence of such a DNA or RNA molecule in the cell or organism.

Thus, a further aspect of the invention relates to cells and organisms (e.g., plant, animal, protozoan, virus, bacterium, or fungus), which comprise at least one expression cassette of the invention, or an recombinant construct or a vector derived therefrom. In certain embodiments, the cell is suspended in culture, while in other embodiments the cell is in, or in part of, a whole organism, such as a microorganism or a plant. The cell can be prokaryotic or of eukaryotic nature. Preferably, the expression cassette or recombinant construct is integrated into the genomic DNA, more preferably within the chromosomal or plastidic DNA, most preferably in the chromosomal DNA of the cell. Accordingly, in one embodiment, the present invention relates to a transformed plant cell, plant or part thereof, or microorganism comprising in its genome at least one stably incorporated expression cassette of the present invention, or a recombinant construct or a vector derived therefrom.

Preferred prokaryotic cells include mainly bacteria such as bacteria of the genus Escherichia, Corynebacterium, Bacillus, Clostridium, Proionibacterium, Butyrivibrio, Eubacterium, Lactobacillus, Erwinia, Agrobacterium, Flavobacterium, Alcaligenes, Phaeodactylum, Colpidium, Mortierella, Entomophthora, Mucor, Crypthecodinium or Cyanobacteria, for example of the genus Synechocystis. Microorganisms which are preferred are mainly those which are capable of infecting plants and thus of transferring the expression cassette or construct of the invention. Preferred microorganisms are those of the genus Agrobacterium and in particular the species Agrobacterium tumefaciens and Agrobacterium rhizogenes.

Eukaryotic cells and organisms comprise plant and animal (preferably non-human) organisms and/or cells and eukaryotic microorganisms such as, for example, yeasts, algae or fungi. Preferred fungi include Aspergillus, Trichoderma, Ashbya, Neurospora, Fusarium, Beauveria or those described in Indian Chem Engr., Section B., 1995, 37(1,2):15, Table 6. Especially preferred is the filamentous Hemiascomycete Ashbya gossypii. Preferred yeasts include Candida, Saccharomyces, Hansenula or Pichia, especially preferred are Saccharomyces cerevisiae or Pichia pastoris (ATCC ii Accession No. 201178). Preferred eukaryotic cells or organisms comprise plant cells and/or organisms, or eukaryotic microorganisms. A corresponding transgenic organism can be generated for example by introducing a desired expression system into a cell derived from such an organism by ways and methods known in the art.

The “plant” as used herein encompasses whole plants, ancestors and progeny of the plants and plant parts, including seeds, shoots, stems, leaves, roots (including tubers), flowers, and tissues and organs, wherein each of the aforementioned comprise the gene/nucleic acid of interest. The term “plant” may also include parts of plants, such as pollen, flowers, kernels, ears, cobs, leaves, husks, stalks, and the like. The term “plant” also encompasses plant cells, plant protoplasts, plant cell tissue cultures, callus tissue, embryos, meristematic regions, gametophytes, sporophytes, pollen and microspores, gamete producing cells, and a cell that regenerates into a whole plant, again wherein each of the aforementioned comprises the gene/nucleic acid of interest.

Plants that are particularly useful in the present invention include all plants which belong to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculenturn, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Malilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgaturn, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Triticosecale rimpaui, Triticum spp. (e.g. Triticum aestivum, Triticum durum, Triticum turgidum, Triticum hybernum, Triticum macha, Triticum sativum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amongst others. Especially preferred are A. thaliana, Nicotiana tabacum, rice, oilseed rape, canola, soybean, corn (maize), cotton, sugarcane, alfalfa, sorghum, and wheat.

“Plant tissue” includes differentiated and undifferentiated tissues or plants, including but not limited to roots, stems, shoots, leaves, pollen, seeds, tumor tissue and various forms of cells and culture such as single cells, protoplast, embryos, and callus tissue. The plant tissue may be in plants or in organ, tissue or cell culture.

Preferably, the organisms are plant organisms. Preferred plants are selected in particular from among crop plants. More preferred plants include, but not limited to, maize, soybean, barley, alfalfa, sunflower, flax, linseed, oilseed rape, canola, sesame, safflower (Carthamus tinctorius), olive tree, peanut, castor-oil plant, oil palm, cacao shrub, or various nut species such as, for example, walnut, coconut or almond, soybean, cotton, peanut, sorghum, tobacco, sugarbeet, sugarcane, rice, wheat, rye, turfgrass, millet, sugarcane, tomato, or potato.

It is noted that a plant need not be considered a “plant variety” simply because it contains stably within its genome a transgene, introduced into a cell of the plant or an ancestor thereof. In addition to a plant, the present invention provides any clone of such a plant, seed, selfed or hybrid progeny and descendants, and any part or propagule of any of these, such as cuttings and seed, which may be used in reproduction or propagation, sexual or asexual. Also encompassed by the invention is a plant which is a sexually or asexually propagated offspring, progeny, clone or descendant of such a plant, or any part or propagule of said plant, offspring, clone or descendant. Genetically modified plants according to the invention, which can be consumed by humans or animals, can also be used as food or feedstuffs, for example directly or following processing known in the art, or be used in biofuel production. The present invention also provides for parts of the organism especially plants, particularly reproductive or storage parts. Plant parts, without limitation, include seed, endosperm, ovule, pollen, roots, tubers, stems, leaves, stalks, fruit, berries, nuts, bark, pods, seeds and flowers.

The expression cassette of the invention, or a recombinant construct or vector derived therefrom, is typically introduced or administered in an amount that allows delivery of at least one copy per cell. Higher amounts (for example at least 5, 10, 100, 500 or 1000 copies per cell) can, if appropriate, result in a more efficient phenotype (e.g., higher expression or higher suppression of the target gene). The amount of the expression cassette, recombinant construct, or vector administered to a cell, tissue, or organism depends on the nature of the cell, tissue, or organism, the nature of the target gene, and the nature of the expression cassette, recombinant construct, or vector, and can readily be optimized to obtain the desired level of expression or inhibition.

Preferably at least about 100 molecules, preferably at least about 1000, more preferably at least about 10,000 of the expression cassette, recombinant construct, or vector, most preferably at least about 100,000 of the expression cassette, recombinant construct, or vector are introduced. In the case of administration of the expression cassette, recombinant construct, or vector to a cell culture or to cells in tissue, by methods other than injection, for example by soaking, electroporation, or lipid-mediated transfection, the cells are preferably exposed to similar levels of the expression cassette, recombinant construct, or vector in the medium.

For example, the expression cassette, recombinant construct, or vector of the invention may be introduced into cells via transformation, transfection, injection, projection, conjugation, endocytosis, and phagocytosis, all are well known in the art. Preferred methods for introduction include, but not limited to:
(a) methods of direct or physical introduction of the expression cassette, recombinant construct, or vector of the invention into the target cell or organism, and
(b) methods of indirect introduction of the expression cassette, recombinant construct, or vector of the invention into the target cell or organism by, for example, a first introduction of a recombinant construct and a subsequent intracellular expression.

### 5. Plant Transformation Techniques

In a further embodiment, the invention provides a method of producing a transgenic plant or plant cell comprising:
(a) transforming a plant or plant cell with at least one aforemetioned expression cassettes, or a recombinant construct or vector derived therefrom, and
(b) optionally regenerating from the plant cell a transgenic plant.

A variety of methods for introducing nucleic acid sequences (e.g., vectors) into the genome of plants and for the regeneration of plants from plant tissues or plant cells are known in the art (Plant Molecular Biology and Biotechnology, Chapter 6-7, pp. 71-119, CRC Press, Boca Raton, Fla., 1993; White F. F., “Vectors for Gene Transfer in Higher Plants,” in Transgenic Plants, Vol. 1, Engineering and Utilization, Kung and Wu, eds., Academic Press, pp. 15-38, 1993; Jenes et al., “Techniques for Gene Transfer,” in Transgenic Plants, Vol. 1, Engineering and Utilization, Kung and Wu, eds., Academic Press, pp. 128-143, 1993; Potrykus, Annu. Rev. Plant Physiol. Plant Mol. Biol., 1991, 42:205-225; Halford et al., Br. Med. Bull., 2000, 56(1):62-73).

5.1 Non-Agrobacterium Transformation

Transformation methods may include direct and indirect methods of transformation. Suitable direct methods include, but not limited to, polyethylene glycol induced DNA uptake, liposome-mediated transformation (U.S. Pat. No. 4,536,475), biolistic methods using the gene gun (Fromm et al., Bio/Technology, 1990, 8(9):833-839; Gordon-Kamm et al., Plant Cell, 1990, 2:603), electroporation, incubation of dry embryos in DNA-comprising solution, and microinjection. In the case of these direct transformation methods, the plasmid used need not meet any particular requirements. Simple plasmids, such as those of the pUC series, pBR322, M13mp series, pACYC184 and the like can be used. If intact plants are to be regenerated from the transformed cells, an additional selectable marker gene is preferably located on the plasmid. The direct transformation techniques are equally suitable for dicotyledonous and monocotyledonous plants.

5.2 Agrobacterium Transformation

Transformation can also be carried out by bacterial infection by means of Agrobacterium (for example EP 0116718), viral infection by means of viral vectors (EP 0067553; U.S. Pat. No. 4,407,956; WO 95/34668; WO 93/03161) or by means of pollen (EP 0270356; WO 85/01856; U.S. Pat. No. 4,684,611). Agrobacterium based transformation techniques (especially for dicotyledonous plants) are well known in the art. The Agrobacterium strain (e.g., Agrobacterium tumefaciens or Agrobacterium rhizogenes) comprises a plasmid (Ti or Ri plasmid) and a T-DNA element which is transferred to the plant following infection with Agrobacterium. The T-DNA (transferred DNA) is integrated into the genome of the plant cell. The T-DNA may be localized on the R1— or Ti-plasmid or is separately comprised in a so-called binary vector. Methods for the Agrobacterium-mediated transformation are described, for example, in Horsch et al., Science, 1985, 227:1229-1231. The Agrobacterium-mediated transformation is best suited to dicotyledonous plants but has also been adopted to monocotyledonous plants. The transformation of plants by Agrobacteria is described in, for example, White F. F., “Vectors for Gene Transfer in Higher Plants,” in Transgenic Plants, Vol. 1, Engineering and Utilization, Kung and Wu, eds., Academic Press, pp. 15-38, 1993; Jenes et al., “Techniques for Gene Transfer,” in Transgenic Plants, Vol. 1, Engineering and Utilization, Kung and Wu, eds., Academic Press, pp. 128-143, 1993; Potrykus, Annu. Rev. Plant Physiol. Plant Mol. Biol., 1991, 42:205-225.

Transformation may result in transient or stable transformation and expression. Although an expression cassette of the present invention can be inserted into any plant and plant cell falling within these broad classes, it is particularly useful in crop plant cells.

Various tissues are suitable as starting material (explant) for the Agrobacterium-mediated transformation process including, but not limited to, callus (U.S. Pat. No. 5,591,616; EP 604662), immature embryos (EP 672752), pollen (U.S. Pat. No. 5,929,300), shoot apex (U.S. Pat. No. 5,164,310), or in planta transformation (U.S. Pat. No. 5,994,624). The method and material described herein can be combined with Agrobacterium mediated transformation methods known in the art.

5.3 Plastid Transformation

In another embodiment, the expression cassette or recombinant construct is directly transformed into the plastid genome. Plastid expression, in which genes are inserted by homologous recombination into the several thousand copies of the circular plastid genome present in each plant cell, takes advantage of the enormous copy number advantage over nuclear-expressed genes to permit high expression levels. In one embodiment, the nucleotide sequence is inserted into a plastid targeting vector and transformed into the plastid genome of a desired plant host. Plants homoplasmic for plastid genomes containing the nucleotide sequence are obtained, and are preferentially capable of high expression of the nucleotide sequence.

Plastid transformation technology is extensively described in, for example, U.S. Pat. No. 5,451,513, U.S. Pat. No. 5,545,817, U.S. Pat. No. 5,545,818, U.S. Pat. No. 5,877,462, WO 95/16783, WO 97/32977, and in McBride et al., Proc. Natl. Acad. Sci. USA, 1994, 91:7301-7305. The basic technique for plastid transformation involves introducing regions of cloned plastid DNA flanking a selectable marker together with the nucleotide sequence into a suitable target tissue, e.g., using biolistic or protoplast transformation (e.g., calcium chloride or PEG mediated transformation). The 1 to 1.5 kb flanking regions, termed targeting sequences, facilitate homologous recombination with the plastid genome and thus allow the replacement or modification of specific regions of the plastome. Initially, point mutations in the chloroplast 16S rRNA and rps12 genes conferring resistance to spectinomycin and/or streptomycin are utilized as selectable markers for transformation (Svab et al., Proc. Natl. Acad. Sci. USA, 1990, 87:8526-8530; Staub et al., Plant Cell, 1992, 4:39-45). The presence of cloning sites between these markers allowed creation of a plastid targeting vector for introduction of foreign genes (Staub et al., EMBO J., 1993, 12:601-606). Substantial increases in transformation frequency are obtained by replacement of the recessive rRNA or r-protein antibiotic resistance genes with a dominant selectable marker, the bacterial aadA gene encoding the spectinomycin-detoxifying enzyme aminoglycoside-3′-adenyltransferase (Svab et al., Proc. Natl. Acad. Sci. USA, 1993, 90:913-917). Other selectable markers useful for plastid transformation are known in the art and encompassed within the scope of the invention.

### 6. Selection and Regeneration Techniques

To select cells which have successfully undergone transformation, it is preferred to introduce a selectable marker which confers, to the cells which have successfully undergone transformation, a resistance to a biocide (for example a herbicide), a metabolism inhibitor such as 2-deoxyglucose-6-phosphate (WO 98/45456) or an antibiotic. The selection marker permits the transformed cells to be selected from untransformed cells (McCormick et al., Plant Cell Reports, 1986, 5:81-84). Suitable selection markers are described above.

Transgenic plants can be regenerated in the known manner from the transformed cells. The resulting plantlets can be planted and grown in the customary manner. Preferably, two or more generations should be cultured to ensure that the genomic integration is stable and hereditary. Suitable methods are described in, for example, Fennell et al., Plant Cell Rep., 1992, 11:567-570; Stoeger et al., Plant Cell Rep., 1995, 14:273-278; and Jahne et al., Theor. Appl. Genet., 1994, 89:525-533.

### 7. Biotechnological Applications

The expression cassettes, and recombinant constructs and vectors derived therefrom, can be applied in biotechnological applications and methods, including but not limited to, optimization of metabolic pathways in, for example, yeasts, fungi or other eukaryotic microorganisms or cells which are used in fermentation for the production of fine chemicals such as amino acids (e.g., lysin or methionin), vitamins (e.g., vitamin B2, vitamin C, vitamin E), carotenoids, oils and fats, polyunsaturated fatty acids, biotin and the like. Depending on the host organisms, the organisms used in the methods of the invention are grown or cultured in a manner with which the skilled worker is familiar.

The expression cassettes, and recombinant constructs and vectors derived therefrom, can also be used to manipulate the production of one or more compounds, such as starch, sugar, lignin, oils and fats, fatty acids and the like in a plant or plant cell. In the context of manipulating lignin biosynthesis for silage or biofuel production, the invention, in another embodiment, provides a method for modifying lignin biosynthesis in a plant comprising:
(a) transforming a plant or plant cell with at least one aforementioned expression cassette, or at least one recombinant construct or vector derived therefrom,
(b) growing said transformed plant or plant cell, and
(c) optionally, regenerating from the plant cell a transgenic plant.

Preferably, expression of the nucleic acid sequence comprised in the aforementioned expression cassettes (e.g., ZmMYB42 coding sequence and/or miR166 precursor sequence) in the transformed and/or regenerated transgenic plant modifies (e.g., down-regulate) the lignin biosynthesis of the transgenic plant, or part thereof, as compared to a corresponding wild-type, non-transformed plant. Methods of transforming a plant or plant cell, selecting transformed plant or plant cell, and regenerating a plant from a plant cell are well known to one skilled in the art in view of the disclosure herein above.

The aforementioned method is also useful for increasing digestibility of a plant. In one embodiment, the increase in digestibility is due to overexpression of a polypeptide that affects the lignin biosynthesis pathway. Examples of such polypeptides include, but not limited to, the maize transcription factor MYB42 (ZmMYB42), or functional variants thereof, as described in, for example, Section 1.2. In another embodiment, the increase in digestibility is due to the production of a miRNA sequence that reduces expression of a particular gene or genes in the lignin biosynthesis pathway. Preferably, such a miRNA sequence is produced from a miR166 precursor sequence that is engineered for the production of such a miRNA sequence. Examples of such miRNA sequences include, but not limited to, the miRNA sequences as described above.

Cell wall digestibility can be assessed by various methods known to one skilled worker. Those methods are developed to measure cell wall digestibility through measuring certain principal components in the forage that contribute energy, such as crude protein (CP), neutral detergent fiber (NDF), fat, and non-fiber carbohydrate (NFC). Examples of such methods include, but not limited to, the acid detergent lignin method and Klason lignin method (see e.g., Jung et al., J. Dairy Sci., 1997, 80:1622-1628), and the methods described in US 2009/0272889.

Cell wall digestibility can also be measured by near infrared relectance spectroscopy (Wilman et al., Animal Feed Science and Technology, 2000, 88:139-151; Mechin et al., Crop Science, 2001, 41:690-697; Frey et al., Crop Science, 2004, 44:1200-1208), by in vitro digestibility (Thomas et al., J. Dairy Sci., 2001, 84:2217-2226; Schwab et al., J. Anim. Feed Sci. Technol., 2003, 109:1-18; MILK2006 for corn silage (University of Wisconsin—Extension, webpage at corn.agronomy.wisc.edu)), or by in situ digestibility (Vanzant et al., J. Anim. Sci., 1998, 76:2717; Harrelson et al., J. Anim. Sci., 2009, 87:2323-2332).

Dry matter yield (DMYLD) and moisture (MST) are measurements used for evaluating silage. DMYLD is one of the parameters used by dairy nutritionists and producers to predict milk yield per acre. Milk yield per acre is one of the indicators for assessing potential profitability for a dairy producer. MST is related to plant maturity which can impact silage quality and dry matter digestibility. MST of silages is determined to ensure that maturities are similar when evaluating digestibility.

Crude Protein (CP) is one of the measurements for determining silage quality. CP is primarily used to provide amino acids to rumen microbes, which degrade carbohydrates in the rumen to produce energy for the animal, and provide amino acids to the animal. CP is one of the parameters that can be used in a summative equation by dairy nutritionists to predict the energy value of silage and other forages.

Acid Detergent Fiber (ADF) is the fiber portion of a plant consisting of cellulose and lignin. Neutral Detergent Fiber (NDF) is ADF plus hemicellulose and is a better measurement of overall plant fiber. ADF has traditionally been associated with digestibility and NDF has been associated with dry matter (DM) intake of forage in ruminants. As ADF and NDF increase, forage digestibility (Neutral Detergent Fiber Digestibility [NDFD] and in vitro dry matter digestibility [IVTDMD]) and dairy cow DM intake typically decrease. However, if NDFD increases, DM intake will also usually increase. DM intake is an important determinant in dairy cow performance for early and mid lactation cows. However, ADF does not account for all of the variation in NDFD (NDF digestibility) (Hoffman et al., Focus on Forage, 2001, 3(10):1-3). NDFD is a better indicator of dairy cow performance than ADF. The increase of one percentage point of NDFD results in an increase of 0.37 lb/day DM intake and a 0.55 lb/day of 4% fat-corrected milk in dairy cows (Oba and Allen, J. Dairy Sci., 1999, 82:589-596). NDFD is determined from the same test used to determine in vitro dry matter digestibility. IVTDMD is an important measurement in evaluating the digestibility of the total DM which can have an impact on DM intake, animal performance, and nutrient utilization, and should validate the results of NDFD.

Plants suitable for the use in the methods of the invention can be monocotyledonous or dicotyledonous plants. In a preferred embodiment, the plant is a monocotyledonous plant, and more preferably, a maize plant, or the plant cell or plant part is from a monocotyledonous plant, preferably a maize plant.

The plant cell, plant or part thereof, that is obtained from the aforementioned methods can be used for production of foodstuff, feedstuff, food supplement, feed supplement, or biofuels. Accordingly, in a further embodiment, the present invention relates to the use of the plant cell, plant or part thereof, obtained according to the aforementioned methods for the preparation of a composition intended for the use in foodstuff, feedstuff, food supplement, or feed supplement. The invention further relates to a method for the preparation of a composition intended for animal or livestock feed comprising the silage of the plant obtained according to the aforemetioned methods, and to the composition intended for animal or livestock feed thus obtained. In a preferred embodiment, said plant is a monocotyledonous plant, and more preferably, a maize plant.

The following examples serve to illustrate certain embodiments and aspects of the present invention and are not to be construed as limiting the scope thereof.

### EXAMPLES

### Example 1

### Design and Construction of Artificial miRNAs Bm3d and Bm3e that Target the ZmCOMT Transcript and Insertion into miR166 Precursor

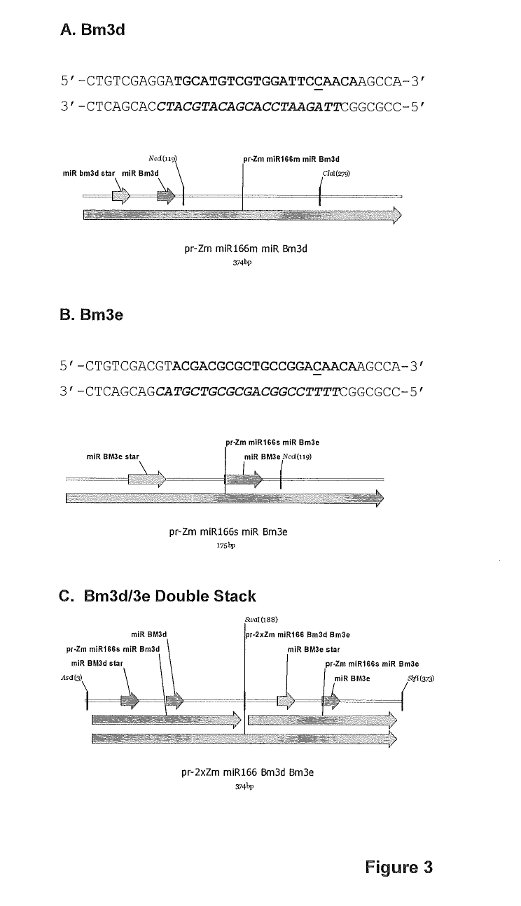

Design of the artificial miRNAs Bm3d (FIG. 3A) and Bm3e (FIG. 3B) targeting the ZmCOMT transcript for down-regulation is described below. To make a Bm3d/3e double stack construct, the two single constructs in their pre-miR backbones were placed in tandem (FIG. 3C).

The ZmCOMT mRNA sequence (SEQ ID NO: 11) including the 5′ and 3′ UTRs was analyzed to identify individual 21-mers along the mRNA that are favorable binding sites for potential artificial miRNAs. These binding site sequences were each given a score (high score was 8) based on a list of eight rules for efficient miRNA design (Weigel rule: Schwab et al., Developmental Cell, 2005, 8:517-527; Reynolds et al., Nat. Biotechnol., 2004, 22:326-330; webpage at wmd3.weigelworld.org/cgi-bin/17731-00035 webapp.cgi?page=Help; project=stdwmd). All 21-mer miRNA binding site sequences with a score of 6 or higher, a T at base pair 12, and an A at base pair 21 were retained.

In order to eliminate 21-mer binding site sequences that are shared among multiple genes, and allow for the design of an artificial miRNA with greater specificity for the intended target, an off-target screen was utilized. This was done by BLAST searching the retained 21-mer miRNA binding site sequences against a maize sequence database to identify homologous maize genes that could become off-targets.

The only 21-mer binding site sequence with a score of 6 or higher that passed the off-target screen begins at base 1452 of the ZmCOMT mRNA sequence in the 3′UTR.

The first two 5′ bases of the 21-mer sequence were changed (this changed the last two 3′ bases of the reverse complement of the binding site which corresponded to the mature miRNA sequence), to avoid transitive RNA silencing of off-target genes. (Parizotto et al., Genes Dev., 2004, 18:2237-2242). The new sequence was run through the off-target screen by BLAST search as described above. Every possible combination of the two bases was made and each version was run through the screen until a 21-mer passed. The 5′ binding site sequence bases of CT were changed to GA so that the 21-mer successfully passed the in silico screen.

The 21-mer binding site sequence was processed through the same off-target screen above except that a human sequence database was used. There were no 100% matches of this 21-mer target sequence to the human database.

This final 21-mer binding site sequence's complementary miRNA was created (Bm3d) as well as its star sequence (FIG. 3A). The star sequence pairs with the miRNA sequence in the hairpin loop structure of the miRNA precursor. The star and miRNA sequences were inserted into the pr-Zm pre-miR166m (374) backbone (US2009/0276921), a maize miR166 precursor backbone comprising 374 nucleotides, to create pr-Zm_miR166m_miR_bm3d (SEQ ID NO: 23).

In order to obtain a second artificial miRNA, the entire process above was repeated, except that the score for efficient miRNA design was lowered to 5.

The only 21-mer binding site sequence with a score of 5 that passed the off-target screen began at base pair 975 of the ZmCOMT mRNA sequence, which placed the binding site of the artificial miRNA in the coding region. The two bases at the 5′ end of the binding site sequence, which were CT, were changed to GA and the 21-mer successfully passed the off-target screen.

This final 21-mer binding site sequence's complementary miRNA was created (Bm3e) as well as its star sequence (FIG. 3B) in the same way as for Bm3d. The Bm3e miRNA and star sequences were inserted into the pr-Zm pre-miR166s (175) backbone (US2009/0276921), a maize miR166 precursor backbone comprising 175 nucleotides, to create pr-Zm_miR166s_miR_bm3e (SEQ ID NO: 25). This backbone was a shorter version of the pr-Zm pre-miR166m (374) backbone. In this version 199 bases were removed from the 3′ end, base pair 32 of the backbone was changed from a G to a C, and base pair 109 was changed from a C to a G.

To design the double stack construct of Bm3d and Bm3e miRNAs, pr-Zm_miR166s_miR_bm3d (SEQ ID NO: 24) and pr-Zm_miR166s_miR_bm3e (SEQ ID NO: 25) were placed in tandem to create pr-2xZm_miR166_bm3d_bm3e (SEQ ID NO: 26). pr-Zm_miR166s_miR_bm3d (SEQ ID NO: 24) was created by inserting the Bm3d miRNA into the pr-Zm pre-miR166s (175) backbone.

### Example 2

### Construction of Expression Cassettes and Vectors

The plasmid vector SB11 (Komari et al., Plant Journal, 1996, 10(1):165-174) was used as the starting base vector. The ZmAHASL2 promoter::ZmAHASL2 gene::ZmAHASL2 3′UTR terminator cassette was inserted between the left border repeat and the right border repeat of the plasmid vector SB11. Acetohydroxyacid synthase, or “AHAS”, and sequences and constructs comprising the AHAS sequences are described in U.S. Pat. No. 6,653,529. The cassettes containing Promoter::Trait gene of interest::Terminator were cloned in order to generate the plasmid vectors for recombination with plasmid vector SB11 prior to plant transformation. The expression cassettes shown in Table 1 were made for corn transformation. These expression cassettes were transformed into a maize inbred line by Agrobacterium-mediated transformation, using AHAS as a selection marker (Fang et al., Plant Molecular Biology, 1992, 18(6):1185-1187).

TABLE 1
List of expression cassettes with artificial miRNAs
designed to target ZmCOMT for down-regulation.

| Construct | Cassette component | SEQ ID NOs |
|---|---|---|
| 1 | p-ZmCOMT:: pr-Zm_miR166m_miR_bm3d::t-NOS | 13, 23, 29 |
| 2 | p-OsCoA-O-methyl::i-Met1-1:: | 7, 31, 23, 10 |
| | pr-Zm_miR166m_miR_bm3d::CoA-O-Methyl_3′UTR | |
| 3 | p-ScBV:: pr-Zm_miR166m_miR_bm3d::t-NOS | 27, 23, 29 |
| 4 | p-ScBV:: pr-Zm_miR166s_miR_bm3e::t-NOS | 27, 25, 29 |
| 5 | p-ScBV254::i-Met1:: pr-Zm_miR166s_miR_bm3e::t-NOS | 28, 31, 25, 29 |
| 6 | p-ScBV254::i-Met1:: pr-Zm_miR166m_miR_bm3d::t-NOS | 28, 31, 23, 29 |
| 7 | p-ZmCOMT::i-Met1:: pr-Zm_miR166m_miR_bm3d::t-NOS | 13, 31, 23, 29 |
| 8 | p-ScBV:: pr-2xZm_miR166_bm3d_bm3e::t-NOS | 27, 26, 29 |
| 9 | p-ScBV254::i-Met1:: pr-2xZm_miR166_bm3d_bm3e::t-NOS | 28, 31, 26, 29 |
| 10 | p-ZmCOMT::i-Met1:: pr-2xZm_miR166_bm3d_bm3e::t-NOS | 13, 31, 26, 29 |

### Example 3

### Plant Transformation

### Maize

Agrobacterium cells harboring a plasmid containing the gene of interest and the mutated maize AHAS gene were grown in YP medium supplemented with appropriate antibiotics for 1-2 days. One loop of Agrobacterium cells was collected and suspended in 1.8 ml M-LS-002 medium (LS-inf). The cultures were incubated while shaking at 1,200 rpm for 5 min-3 hrs. Corn cobs were harvested at 8-11 days after pollination. The cobs were sterilized in 20% Clorox solution for 5 min, followed by spraying with 70% Ethanol and then thoroughly rinsing with sterile water. Immature embryos 0.8-2.0 mm in size were dissected into the tube containing Agrobacterium cells in LS-inf solution.

Agrobacterium infection of the embryos was carried out by inverting the tube several times. The mixture was poured onto a filter paper disk on the surface of a plate containing co-cultivation medium (M-LS-011). The liquid agro-solution was removed and the embryos were checked under a microscope and placed scutellum side up. Embryos were cultured in the dark at 22° C. for 2-4 days, and were transferred to M-MS-101 medium without selection and incubated for four to seven days. Embryos were then transferred to M-LS-202 medium containing 0.75 μM imazethapyr and grown for four weeks at 27° C. to select for transformed callus cells.

Plant regeneration was initiated by transferring resistant calli to M-LS-504 medium supplemented with 0.75 μM imazethapyr and grown under light at 26° C. for two to three weeks. Regenerated shoots were then transferred to a rooting box with M-MS-618 medium (0.5 μM imazethapyr). Plantlets with roots were transferred to soil-less potting mixture and grown in a growth chamber for a week, then transplanted to larger pots and maintained in a greenhouse until maturity.

Transgenic maize plant production is also described, for example, in U.S. Pat. No. 5,591,616 and WO/2006136596, both of which are hereby incorporated by reference in their entirety. Transformation of maize may be made using Agrobacterium transformation, as described in U.S. Pat. Nos. 5,591,616; 5,731,179; 5,981,840; 5,990,387; 6,162,965; 6,420,630, U.S. patent application publication number 2002/0104132, and the like. Transformation of maize (Zea mays L.) can also be performed with a modification of the method described by Ishida et al. (Nature Biotech., 1996, 14:745-750). The inbred line A188 (University of Minnesota) or hybrids with A188 as a parent are good sources of donor material for transformation (Fromm et al., Biotech, 1990, 8:833), but other genotypes can be used successfully as well. Ears are harvested from corn plants at approximately 11 days after pollination (DAP) when the length of immature embryos is about 1 to 1.2 mm. Immature embryos are co-cultivated with Agrobacterium tumefaciens that carry “super binary” vectors and transgenic plants are recovered through organogenesis. The super binary vector system is described in WO 94/00977 and WO 95/06722. Vectors are constructed as described. Various selection marker genes are used including the maize gene encoding a mutated acetohydroxy acid synthase (AHAS) enzyme (U.S. Pat. No. 6,025,541). Similarly, various promoters are used to regulate the trait gene to provide constitutive, developmental, inducible, tissue or environmental regulation of gene transcription.

Excised embryos can be used and can be grown on callus induction medium, then maize regeneration medium, containing imidazolinone as a selection agent. The petri dishes are incubated in the light at 25° C. for 2-3 weeks, or until shoots develop. The green shoots are transferred from each embryo to maize rooting medium and incubated at 25° C. for 2-3 weeks, until roots develop. The rooted shoots are transplanted to soil in the greenhouse. T1 seeds are produced from plants that exhibit tolerance to the imidazolinone herbicides and which are PCR positive for the transgenes.

### Wheat

A specific example of wheat transformation can be found in PCT Application No. WO 93/07256. Transformation of wheat can also be performed with the method described by Ishida et al. (Nature Biotech., 1996, 14:745-750). The cultivar Bobwhite (available from CYMMIT, Mexico) is commonly used in transformation. Immature embryos are co-cultivated with Agrobacterium tumefaciens that carry “super binary” vectors, and transgenic plants are recovered through organogenesis. The super binary vector system is described in WO 94/00977 and WO 95/06722, which are hereby incorporated by reference in its entirety. Vectors are constructed as described. Various selection marker genes can be used including the maize gene encoding a mutated acetohydroxy acid synthase (AHAS) enzyme (U.S. Pat. No. 6,025,541). Similarly, various promoters can be used to regulate the trait gene to provide constitutive, inducible, developmental, tissue or environmental regulation of gene transcription.

After incubation with Agrobacterium, the embryos are grown on callus induction medium, then regeneration medium, containing imidazolinone as a selection agent. The petri dishes are incubated in the light at 25° C. for 2-3 weeks, or until shoots develop. The green shoots are transferred from each embryo to rooting medium and incubated at 25° C. for 2-3 weeks, until roots develop. The rooted shoots are transplanted to soil in the greenhouse. T1 seeds are produced from plants that exhibit tolerance to the imidazolinone herbicides and which are PCR positive for the transgenes.

### Rice

Rice may be transformed using methods disclosed in U.S. Pat. Nos. 4,666,844; 5,350,688; 6,153,813; 6,333,449; 6,288,312; 6,365,807; 6,329,571, and the like.

### Example 4

### Artificial miRNA Bm3d miRNA Detection

4.1. Bm3d miRNA Detection.

In order to determine if the artificial miRNA Bm3d was diced out of its pre-miR backbone and processed into the mature species in planta, a 3′ adaptor was ligated to the mature miRNA, then cloned and sequenced. Properly processed miRNA was confirmed by PCR. Total RNA extracted from maize leaf tissue transformed with one of the constructs carrying the artificial miRNA Bm3d (constructs 1, 2, and, 3) was evaluated. Maize lines transformed with construct 3 were further analyzed.

RNA samples extracted from transgenic maize with construct 1 and construct 2 did not show an expected band from the RT-PCR reactions, but construct 3 showed the expected band (52 base pairs). The absence of the expected band in constructs 1 and 2 was probably due to a weak promoter activity by ZmCOMT and OsCCoAOMT promoters, specifically at the developmental time of tissue sampling. The artificial miRNA Bm3d pre-miR transcript was driven by the ScBV promoter in construct 3, by the ZmCOMT promoter in construct 1, and by the OsCCoAOMT promoter with intron i-Met1-1 in construct 2.

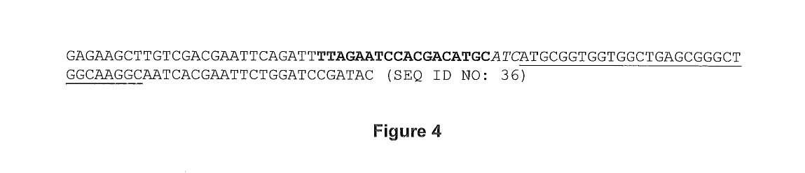

The sequencing results and alignment for construct 3 indicated that the inserts had the expected sequence (FIG. 4). This result confirms the expression of the pre-miR transcript as well as its successful processing into the mature miRNA Bm3d in transgenic maize transformed with construct 3.

4.2. Bm3d miRNA Detection (ABI Taqman Assay Method).

The ABI Taqman assay method for miRNA detection is known to be more sensitive than the 3′ adaptor method. Therefore, the ABI Taqman assay was used to detect the artificial miRNA Bm3d in construct 1 and construct 2 as well as to confirm the results for construct 3 by the 3′ adaptor method. A custom designed ABI assay kit was used to amplify the artificial miRNA Bm3d using the sequence ABI Bm3d (SEQ ID NO: 40).

The ABI Taqman assay method to detect miRNAs was a qRT-PCR experiment. The data from all qRT-PCR reactions were normalized to the internal ZmActin1 control and given relative expression level values (Table 2). The artificial miRNA Bm3d was detected in all of the transgenic lines tested that were transformed with the Bm3d miRNA construct. Relative expression levels of Bm3d miRNA were very high for plants transformed with construct 3 and comparatively much lower in plants transformed with construct 1. The data agreed with the results for Bm3d miRNA detection by the 3′ adaptor method described above.

TABLE 2
Relative expression levels of the artificial
miRNA Bm3d in plants of T1 generation.

| | | Event | Rel. Exp. Level of | Standard |
|---|---|---|---|---|
| Construct | Promoter | Name | Bm3d miRNA | Deviation |
| 1 | ZmCOMT | 1A | 0.84 | 0.516 |
| | | 1B | 0.41 | 0.114 |
| | | 1C | 0.33 | 0.052 |
| | | 1D | 0.18 | 0.011 |
| | | 1E | 0.68 | 0.154 |
| | | 1F | 5.29 | 1.093 |
| | | 1G | 0.66 | 0.157 |
| | | 1H | 1.62 | 0.236 |
| 2 | OsCCoAOMT | 2I | 0.13 | 0.078 |
| | | 2J | 0.89 | 0.600 |
| | | 2K | 0.22 | 0.117 |
| 3 | ScBV | 3L | 642.5 | 380.762 |
| | | 3M | 366.99 | 183.404 |
| | | 3N | 182.89 | 89.278 |

The expression level was normalized to ZmActin1 for each of the RNA samples used. Each data point is derived from an average of three replicates.

### Example 5

### COMT Expression Analysis by qRT-PCR

Maize leaf tissue of the second fully developed leaf from the top was harvested around the vegetative developmental stages V7-V8, quickly frozen in liquid nitrogen, and kept frozen in −80° C. until used. Leaf tissues were crushed to a rough powder on dry ice and poured into wells of a 96-well block with ball bearings.

Expression analysis for ZmCOMT was carried out by extracting RNA from the leaf tissue and performing qRT-PCR. The data were normalized to the expression level of the internal control ZmGAPDH (Glyceraldehyde-3-phosphate dehydrogenase) and then compared with WT controls. The difference between normalized transgenic events and normalized WT expression of ZmCOMT was calculated. The results for the expression analysis (Table 3) clearly show that ZmCOMT mRNA expression was down-regulated in transgenic plants.

TABLE 3
Expression of ZmCOMT measured by qRT-PCR in T1 or T0 generation
transgenic plants relative to the wild type (WT) control.

| | | | COMT Expression (% WT control) | | |
|---|---|---|---|---|---|
| | | | | Observed | |
| | | No. of | Construct | Range | Standard |
| Construct | Generation | events | Average | (Min-Max) | deviation |
| 1 | T1 | 8 | 53.68 | 33.12-76.46 | 17.19 |
| 2 | T1 | 3 | 61.36 | 45.69-73.27 | 19.80 |
| 3 | T1 | 8 | 11.31 | 8.31-21.09 | 4.62 |
| 4 | T0 | 9 | 9.71 | 3.20-23.13 | 6.81 |
| 5 | T0 | 13 | 12.65 | 1.37-27.80 | 8.03 |
| 6 | T0 | 10 | 22.93 | 5.14-42.40 | 11.05 |
| 7 | T0 | 7 | 48.37 | 31.31-70.58 | 17.00 |
| 8 | T0 | 17 | 5.94 | 1.57-17.32 | 5.13 |
| 9 | T0 | 2 | 7.78 | 7.27-8.27 | 0.70 |
| 10 | T0 | 9 | 46.73 | 6.73-78.96 | 23.18 |

The expression levels in T1 generation plants are the averages of qRT-PCR results from three or more T1 plants of each event. The expression levels in T0 plants are the average of two qRT-PCR results from one plant of each event.

### Example 6

### Digestibility Test

T1 plants were grown in the greenhouse. At 30 DAP (days after pollination), stems (3 internodes above the node with ear shank to 3 internodes below it; sheath and ears removed) and leaves (3 leaves including the ear leaf and 2 above) were sampled from each plant. Samples from up to 3 plants were pooled for each transgenic event. Internodes were frozen in liquid nitrogen, and leaves were stored in a container with dry ice. All samples were kept frozen until analyzed.

The NRC (National Research Council) technique was used to measure neutral detergent fiber (NDF) (Nutrient Requirements of Dairy Cows, 7th Revised Edition, 2001), following AOAC (Association of Official Analytical Chemists) methodology for in situ performance with fiber products to better obtain real feeding value for fiber digestibility within the rumen of lactating dairy cows (Vanzant et al., J. Anim. Sci., 1998, 76:2717).

The test results are summarized in Table 4A. Down-regulation of ZmCOMT expression by miRNA Bm3d produced NDF digestibility (DNFD) of leaf biomass up to 52% in construct 1 with the ZmCOMT promoter, 41% in construct 2 with the OsCCoAOMT promoter, and 40% in construct 3 with ScBV promoter. The 52% NDFD is a significant increase compared to 38% in non-transgenic control sample. In stem biomass, NDFD increases were as great as 63% with construct 1, 62% with construct 2, and 77% with construct 3. Although no control NDFD data were available for stem biomass, the increases are expected to be significant, considering that stem digestibility is typically lower than leaf digestibility.

TABLE 4A
In situ digestibility tests of stem and leaf tissues (unit = % DM).

| | | Stem | | Leaf | |
|---|---|---|---|---|---|
| | | NDF | NDFD | NDF | NDFD |
| Construct | Event | (% DM) | (% DM) | (% DM) | (% DM) |
| 1 | 1H | 43.87 | 35.47 | 47.87 | 41.64 |
| | 1A | 44.23 | 63.33 | 50.72 | 52.34 |
| | 1G | 49.40 | 45.90 | 49.69 | 44.08 |
| | 1C | 43.71 | 45.00 | 52.52 | 48.05 |
| | 1F | 43.06 | 53.37 | 45.54 | 33.23 |
| | 1B | 42.50 | 47.29 | 49.28 | 39.78 |
| | 1D | 41.94 | 39.47 | 45.67 | 34.01 |
| | 1E | 41.51 | 57.39 | 46.68 | 40.52 |
| 2 | 2I | 42.10 | 53.68 | 45.87 | 41.66 |
| | 2J | 41.27 | 49.52 | 46.94 | 37.02 |
| | 2K | 42.41 | 62.10 | 44.95 | 24.70 |
| 3 | 3O | 38.43 | 66.64 | 48.86 | 35.05 |
| | 3P | 39.05 | 77.16 | 47.17 | 31.95 |
| | 3Q | 40.31 | 58.86 | 49.23 | 35.52 |
| | 3M | 40.92 | 50.17 | 49.87 | 35.15 |
| | 3L | 39.44 | 57.69 | 48.21 | 40.92 |
| | 3N | 39.52 | 52.32 | 47.57 | 36.46 |
| | 3R | 40.05 | 48.75 | 46.34 | 38.10 |
| | 3S | NA | NA | 47.89 | 35.84 |

| | | | | |
|---|---|---|---|---|
| Control (non-transgenic) | NA | NA | 46.20 | 38.23 |

NDF = Neutral detergent fiber, NDFD = 20-h digestibility of NDF, DM = Dry matter.

Several stem samples that showed a high level of enhanced digestibility from in situ digestibility (Table 4A) were further tested by an in vitro digestibility test measured by wet chemistry (Thomas et al., J. Dairy Sci., 2001, 84:2217-2226; Schwab et al., J. Anim. Feed Sci. Technol., 2003, 109:1-18; Feed and Forage Analaysis, University of Wisconsin Soil and Plant Analysis Lab, webpage at uwlab.soils.wisc.edu/madison/index.htm? . . . /procedures.htm&contents.asp?menu=2). The results, as summarized in Table 4B, support that stem tissues of selected transgenic events show improved NDF digestibility compared to the control. Improved digestibility typically results in increased DM intake and fat-corrected milk production in cattle.

TABLE 4B
In vitro digestibility tests of stem tissues
measured by wet chemistry (unit = % DM).

| | | | | Difference in |
|---|---|---|---|---|
| | | NDF | NDFD30 | NDFD30 (event |
| Construct | Event | (% DM) | (% DM) | vs. control) |
| 1 | 1A | 43.37 | 40.58 | 17.71 |
| 2 | 2K | 42.86 | 41.90 | 19.03 |
| 3 | 3O | 41.00 | 56.59 | 33.72 |
| | 3P | 43.39 | 49.94 | 27.07 |

| | | |
|---|---|---|
| Control (non-transgenic) | 47.05 | 22.87 |

NDF = Neutral detergent fiber, NDFD30 = 30-h digestibility of NDF, DM = Dry matter.

### Example 7

### In Vitro Digestibility of T1 Plants

T1 plants were grown in the field. Up to five (5) plants of hemizygous transgenic events and nulls (control), respectively, were harvested at 35 days after pollination. Null controls are also T1 plants but contain no transgene. Stalk samples consisted of the four internodes above and three internodes below the ear node. Leaves from those internodes were sampled separately. In vitro NDFD at 30 hr (NDFD30) was measured by standard NIRS (near-infrared spectroscopy). The test results are summarized in Table 5A.

TABLE 5A
In vitro digestibility tests of stalk and leaf tissues measured by NIRS (unit = % DM).

| | | Stalk | | Leaf | | Difference in NDFD30 | |
|---|---|---|---|---|---|---|---|
| | | NDF | NDFD30 | NDF | NDFD30 | (Event vs. Null) | |
| Construct | Event | (% DM) | (% DM) | (% DM) | (% DM) | Stalk | Leaf |
| 4 | 4T | 45.27 | 36.18 | 53.04 | 43.38 | 4.93 | 4.89 |
| | 4T (null) | 47.77 | 31.25 | 51.59 | 38.49 | | |
| | 4U | 44.39 | 36.57 | 53.4 | 39.12 | 7.51 | 3.28 |
| | 4U (null) | 45.46 | 29.06 | 51.73 | 35.84 | | |
| | 4V | 45 | 36.28 | 53.44 | 36.07 | 5.43 | NA |
| | 4V (null) | 47.84 | 30.85 | | | | |
| | 4W | 49.38 | 37.33 | 56.69 | 41.06 | 8.89 | 1.85 |
| | 4W (null) | 44.79 | 28.44 | 50.95 | 39.21 | | |
| | 4X | 46.16 | 34.97 | 53.98 | 40.98 | 6.40 | 5.46 |
| | 4X (null) | 48.96 | 28.57 | 53.51 | 35.52 | | |
| 5 | 5Z | 46.87 | 30.68 | 54.62 | 34.72 | 3.90 | −6.03 |
| | 5Z (null) | 44.44 | 26.78 | 49.14 | 40.75 | | |
| | 5A1 | 44.06 | 35.21 | 52.37 | 37.33 | 7.40 | −6.22 |
| | 5A1 (null) | 42.01 | 27.81 | 47.99 | 43.55 | | |
| | 5B1 | 40.48 | 28.65 | 49.74 | 40.04 | −1.07 | 3.82 |
| | 5B1 (Null) | 44.57 | 29.72 | 52.36 | 36.22 | | |
| | 5C1 | 47.21 | 31.96 | 54.46 | 34.54 | 3.42 | −2.80 |
| | 5C1 (null) | 46.89 | 28.54 | 53.02 | 37.34 | | |
| | 5D1 | 42.46 | 30.54 | 52.22 | 41.46 | 0.61 | 4.70 |
| | 5D1 (null) | 46.02 | 29.93 | 52.01 | 36.76 | | |
| | 5E1 | 47.68 | 30.64 | 53.3 | 34.36 | 2.90 | −4.43 |
| | 5E1 (null) | 45.9 | 27.74 | 51.24 | 38.79 | | |
| | 5F1 | 46.8 | 33.26 | 52.69 | 41.37 | 3.07 | 1.05 |
| | 5F1 (null) | 45.81 | 30.19 | 52.75 | 40.32 | | |
| 6 | 6G1 | 44.07 | 30.63 | 50.9 | 41.24 | −0.55 | 3.11 |
| | 6G1 (null) | 46.87 | 31.18 | 54 | 38.13 | | |
| | 6H1 | 48.35 | 29.66 | 53.64 | 37.51 | −3.11 | −1.32 |
| | 6H1 (null) | 46.07 | 32.77 | 52.75 | 38.83 | | |
| | 6I1 | 53.33 | 28.87 | 54.94 | 33.23 | −2.12 | −4.35 |
| | 6I1 (null) | 48.35 | 30.99 | 52.41 | 37.58 | | |
| | 6J1 | 42.47 | 27.96 | 49.25 | 45.03 | −0.25 | 2.30 |
| | 6J1 (null) | 46.04 | 28.21 | 50.1 | 42.73 | | |
| | 6K1 | 45.23 | 33.22 | 51.44 | 37.44 | 5.59 | NA |
| | 6K1 (null) | 44.59 | 27.63 | | | | |
| | 6L1 | 45.39 | 29.86 | 49.59 | 41.30 | NA | −0.44 |
| | 6L1 (null) | | | 50.38 | 41.74 | | |
| | 6M1 | 48.8 | 27.00 | 51.21 | 35.45 | −0.21 | −1.08 |
| | 6M1 (null) | 46.21 | 27.21 | 51.32 | 36.53 | | |
| | 6N1 | 43.67 | 30.95 | 49.67 | 43.95 | 0.64 | 5.74 |
| | 6N1 (null) | 42.62 | 30.31 | 49.86 | 38.21 | | |
| | 6O1 | 42.93 | 26.65 | 48.96 | 45.64 | −2.16 | 7.84 |
| | 6O1 (null) | 46.55 | 28.81 | 52.39 | 37.80 | | |
| | 6P1 | 47.77 | 30.41 | 53.51 | 32.64 | 1.53 | −1.07 |
| | 6P1 (null) | 48.71 | 28.88 | 54.09 | 33.71 | | |
| | 6Q1 | 44.91 | 27.76 | 49.59 | 43.42 | −2.39 | 6.24 |
| | 6Q1 (null) | 45.82 | 30.15 | 52.1 | 37.18 | | |
| | 6R1 | 50.88 | 28.85 | 53.93 | 36.65 | −1.48 | −2.10 |
| | 6R1 (null) | 47.43 | 30.33 | 52.47 | 38.75 | | |
| 7 | 7S1 | 49.79 | 30.95 | 53.97 | 35.51 | 3.32 | −2.84 |
| | 7S1 (null) | 38.35 | 38.35 | 38.35 | 38.35 | | |
| | 7T1 | 45.17 | 26.00 | 50.06 | 39.45 | −1.38 | −7.49 |
| | 7T1 (null) | 46.11 | 27.38 | 52.86 | 46.94 | | |
| | 7U1 | 46.68 | 25.86 | 50.73 | 37.73 | −4.35 | −3.08 |
| | 7U1 (null) | 43.94 | 30.21 | 48.65 | 40.81 | | |
| | 7V1 | 47.94 | 25.25 | 51.74 | 35.24 | −2.80 | −1.06 |
| | 7V1 (null) | 43.9 | 28.05 | 50.12 | 36.30 | | |
| | 7W1 | 46.65 | 25.31 | 49.23 | 36.74 | −5.26 | −4.13 |
| | 7W1 (null) | 46.05 | 30.57 | 50.24 | 40.87 | | |
| | 7X1 | 45.7 | 23.97 | 50.04 | 38.55 | −2.01 | −1.22 |
| | 7X1 (null) | 46.19 | 25.98 | 49.68 | 39.77 | | |
| | 7Y1 | 47.19 | 25.42 | 50.08 | 39.87 | −2.89 | −0.92 |
| | 7Y1 (null) | 48.3 | 28.31 | 51.9 | 40.79 | | |
| | 7Z1 | 46.25 | 26.45 | 51.59 | 38.80 | −1.84 | 3.47 |
| | 7Z1 (null) | 45.96 | 28.29 | 51.74 | 35.33 | | |
| | 7A2 | 48.41 | 28.63 | 52.64 | 36.48 | −5.23 | −6.05 |
| | 7A2 (null) | 41.82 | 33.86 | 49.28 | 42.53 | | |
| 8 | 8B2 | 46.2 | 41.36 | 53.87 | 41.01 | 9.30 | 2.16 |
| | 8B2 (null) | 46.06 | 32.06 | 53.45 | 38.85 | | |
| | 8C2 | 42.93 | 35.70 | 51.85 | 43.31 | 2.66 | 6.18 |
| | 8C2 (null) | 47.49 | 33.04 | 52.78 | 37.13 | | |
| | 8D2 | 48.34 | 42.82 | 56.75 | 38.49 | 14.66 | 1.54 |
| | 8D2 (null) | 45.48 | 28.16 | 51.73 | 36.95 | | |
| | 8E2 | 41.42 | 39.42 | 50.63 | 45.75 | 11.38 | 6.29 |
| | 8E2 (null) | 49.36 | 28.04 | 51.66 | 39.46 | | |
| | 8F2 | 44.08 | 37.95 | 50.03 | 43.91 | 5.38 | 7.03 |
| | 8F2 (null) | 47.83 | 32.57 | 52.51 | 36.88 | | |
| | 8G2 | 42.93 | 38.65 | 53.62 | 49.30 | 3.94 | 8.59 |
| | 8G2 (null) | 48.75 | 34.71 | 53.5 | 40.71 | | |
| | 8H2 | 46.28 | 44.31 | 52.89 | 43.50 | 17.08 | 6.77 |
| | 8H2 (null) | 48.91 | 27.23 | 53.02 | 36.73 | | |
| | 8I2 | 45.82 | 42.56 | 53.59 | 46.63 | 5.88 | 6.07 |
| | 8I2 (null) | 46.92 | 36.68 | 53.53 | 40.56 | | |
| | 8J2 | 43.82 | 39.72 | 52.38 | 44.79 | 9.42 | 7.92 |
| | 8J2 (null) | 44.71 | 30.30 | 50.42 | 36.87 | | |
| | 8K2 | 45.03 | 40.34 | 52.86 | 43.99 | 10.42 | 9.85 |
| | 8K2 (null) | 49.55 | 29.92 | 52.25 | 34.14 | | |
| 9 | 9L2 | 47.51 | 35.82 | 52.65 | 38.05 | 6.03 | −4.30 |
| | 9L2 (null) | 45.63 | 29.79 | 51.73 | 42.35 | | |
| | 9M2 | 44.26 | 34.08 | 51.1 | 37.65 | 4.53 | −4.35 |
| | 9M2 (null) | 45.4 | 29.55 | 50.62 | 42.00 | | |
| 10 | 10N2 | 44.53 | 23.15 | 47.25 | 40.65 | −2.81 | −0.93 |
| | 10N2 (null) | 43.61 | 25.96 | 48.39 | 41.58 | | |
| | 10O2 | 45 | 28.65 | 49.99 | 39.53 | −0.08 | −0.41 |
| | 10O2 (null) | 46.97 | 28.73 | 51.51 | 39.94 | | |
| | 10P2 | 43.58 | 27.29 | 50.32 | 44.51 | −4.31 | 1.54 |
| | 10P2 (null) | 48.23 | 31.60 | 52.43 | 42.97 | | |
| | 10Q2 | 46.79 | 25.07 | 50.32 | 45.52 | −6.13 | 7.19 |
| | 10Q2 (null) | 47.95 | 31.20 | 50.97 | 38.33 | | |
| | 10R2 | 46.06 | 27.57 | 49.76 | 38.38 | −2.93 | 5.35 |
| | 10R2 (null) | 49.17 | 30.50 | 52.62 | 33.03 | | |
| | 10S2 | 45.6 | 28.67 | 50.5 | 39.35 | 1.43 | −1.88 |
| | 10S2 (null) | 42.49 | 27.24 | 49.09 | 41.23 | | |
| | 10T2 | 45.18 | 27.42 | 48.28 | 40.15 | −3.35 | −0.53 |
| | 10T2 (null) | 44.98 | 30.77 | 50.01 | 40.68 | | |
| | 10U2 | 48.66 | 31.53 | 53.05 | 30.29 | 3.69 | −5.63 |
| | 10U2 (null) | 47.08 | 27.84 | 52.03 | 35.92 | | |
| | 10V2 | 47.67 | 25.57 | 50.26 | 36.88 | 3.25 | −0.54 |
| | 10V2 (null) | 43.56 | 22.32 | 49.02 | 37.42 | | |

NDF = Neutral detergent fiber, NDFD30 = 30-h digestibility of NDF.

Several stalk tissue samples that showed enhanced digestility from in vitro digestibility measured by NIRS (Table 5A) were also tested by in vitro digestibility test measured by wet chemistry (Thomas et al., J. Dairy Sci., 2001, 84:2217-2226; Schwab et al., J. Anim. Feed Sci. Technol., 2003, 109:1-18; Feed and Forage Analaysis, University of Wisconsin Soil and Plant Analysis Lab, website at uwlab.soils.wisc.edu/madison/index.htm?./procedures.htm&contents.asp?Menu=2). The results, as summarized in Table 5B, provide support for stalk tissues of several events producing improved NDF digestibility compared to the null control, consistent with results measure by NIRS (Table 5A), in particular for events of contruct 8. Improved digestibility typically results in increased DM intake and fat-corrected milk production in cattle.

TABLE 5B
In vitro digestibility tests of stalk tissues
measured by wet chemistry (unit = % DM).

| | | | | Difference in |
|---|---|---|---|---|
| | | NDF | NDFD30 | NDFD30 (Event |
| Construct | Event | (% DM) | (% DM) | vs Null) |
| 4 | 4X | 45.81 | 48.79 | 3.08 |
| | 4X (null) | 52.49 | 45.70 | |
| 5 | 5C1 | 54.29 | 47.74 | 8.17 |
| | 5C1 (null) | 52.56 | 39.57 | |
| 6 | 6H1 | 50.34 | 40.90 | 3.88 |
| | 6H1 (null) | 50.21 | 37.02 | |
| | 6Q1 | 48.55 | 38.41 | −7.07 |
| | 6Q1 (null) | 51.76 | 45.48 | |
| 7 | 7T1 | 49.76 | 38.28 | −8.99 |
| | 7T1 (null) | 48.06 | 47.27 | |
| | 7U1 | 50.34 | 45.87 | −5.10 |
| | 7U1 (null) | 46.32 | 50.97 | |
| 8 | 8D2 | 51.06 | 55.88 | 15.27 |
| | 8D2 (null) | 50.12 | 40.60 | |
| | 8E2 | 43.19 | 52.14 | 15.94 |
| | 8E2 (null) | 51.49 | 36.20 | |
| | 8J2 | 44.68 | 62.29 | 25.93 |
| | 8J2 (null) | 47.55 | 36.36 | |
| 9 | 9L2 | 54.38 | 51.62 | 20.52 |
| | 9L2 (null) | 47.62 | 31.10 | |
| 10 | 10O2 | 48.31 | 38.56 | 7.61 |
| | 10O2 (null) | 48.06 | 30.95 | |

NDF = Neutral detergent fiber, NDFD30 = 30-h digestibility of NDF.

### Example 8

### In Vitro Digestibility of F1 Hybrid Plants

Two F1 hybrids from crosses of each transgenic event with two testers (nutritionally enhanced and yellow dent) were grown in two field locations with six replications per location in a randomized complete block design. Six whole plants were collected for whole plant analysis. Stem sections from six additional plants were collected for stalk segment analysis. These stem sections consisted of the four internodes above and three internodes below the ear node. No major negative influences on the crop due to weeds, disease or insects were noted. Harvest was conducted at one half to two thirds milk line. In vitro NDFD at 30 hr (NDFD30) was measured by standard NIRS (near-infrared spectroscopy). The test results are summarized in Table 6A.

TABLE 6A
In vitro digestibility of whole plant and stalk segments by NIRS (Unit = % DM).

| | | Tester 1 (nutritionally enhanced) | | | | Tester 2 (yellow dent) | | | |
|---|---|---|---|---|---|---|---|---|---|
| | | Whole Plant | | Stalk Segment | | Whole Plant | | Stalk Segment | |
| | | NDF | NDFD30 | NDF | NDFD30 | NDF | NDFD30 | NDF | NDFD30 |
| Construct | Description | (% DM) | (% DM) | (% DM) | (% DM) | (% DM) | (% DM) | (% DM) | (% DM) |
| 1 | Control (C) | 40.6 | 48.7 | 60.9 | 29 | 42.5 | 49.9 | 66.2 | 26.5 |
| | Event 1C (T) | 41.3 | 48.4 | 61.5 | 28.4 | 40.8 | 52.2 | 64.7 | 26.7 |
| | T − C | 0.7 | −0.3 | 0.7 | −0.7 | −1.8 | 2.3 | −1.4 | 0.3 |
| | (T/C)% | 102 | 99 | 101 | 98 | 96 | 105 | 98 | 101 |
| | p-value | 0.75 | 0.86 | 0.66 | 0.73 | 0.45 | 0.13 | 0.47 | 0.83 |
| | Event 1D (T) | 39.4 | 47.9 | 60.5 | 30 | 38.6 | 52.5 | 66.4 | 26.4 |
| | T − C | −1.2 | −0.8 | −0.3 | 1 | −3.9 | 2.6 | 0.2 | −0.1 |
| | (T/C)% | 97 | 98 | 99 | 103 | 91 | 105 | 100 | 100 |
| | p-value | 0.5 | 0.66 | 0.79 | 0.61 | 0.07 | 0.03 | 0.89 | 0.97 |
| | Event 1A (T) | 38.6 | 49.1 | 64.4 | 27.8 | 41.4 | 52 | 65.4 | 28 |
| | T − C | −2 | 0.5 | 3.5 | −1.2 | −1.1 | 2.1 | −0.7 | 1.5 |
| | (T/C)% | 95 | 101 | 106 | 96 | 97 | 104 | 99 | 106 |
| | p-value | 0.3 | 0.78 | 0.02 | 0.53 | 0.64 | 0.15 | 0.66 | 0.24 |
| | Event 1G (T) | 39.5 | 47.6 | 62.7 | 27.5 | 41.3 | 51.8 | 66.5 | 26.4 |
| | T − C | −1 | −1.1 | 1.9 | −1.6 | −1.2 | 1.9 | 0.4 | −0.1 |
| | (T/C)% | 97 | 98 | 103 | 95 | 97 | 104 | 101 | 100 |
| | p-value | 0.63 | 0.59 | 0.17 | 0.47 | 0.58 | 0.12 | 0.84 | 0.96 |
| | Event 1E (T) | 38.2 | 49.1 | 63.9 | 28.8 | 42.7 | 51.2 | 61.5 | 27.5 |
| | T − C | −2.4 | 0.5 | 3.1 | −0.3 | 0.1 | 1.3 | −4.7 | 1 |
| | (T/C)% | 94 | 101 | 105 | 99 | 100 | 103 | 93 | 104 |
| | p-value | 0.22 | 0.79 | 0.06 | 0.9 | 0.95 | 0.37 | 0.01 | 0.45 |
| | Event 1H (T) | 41.5 | 44.9 | 53.7 | 32.4 | 42.6 | 48.8 | 57.3 | 30.9 |
| | T − C | 0.9 | −3.8 | −7.1 | 3.3 | 0.1 | −1.2 | −8.8 | 4.5 |
| | (T/C)% | 102 | 92 | 88 | 112 | 100 | 98 | 87 | 117 |
| | p-value | 0.63 | 0.01 | 0 | 0.1 | 0.96 | 0.35 | 0 | 0 |
| | Event 1B (T) | 38.6 | 49.1 | 59.1 | 29 | 42.8 | 50.2 | 64.8 | 27.3 |
| | T − C | −2 | 0.4 | −1.7 | 0 | 0.3 | 0.2 | −1.4 | 0.9 |
| | (T/C)% | 95 | 101 | 97 | 100 | 101 | 100 | 98 | 103 |
| | p-value | 0.38 | 0.78 | 0.31 | 1 | 0.91 | 0.85 | 0.4 | 0.48 |
| 2 | Control (C) | 43.1 | 50.5 | 63.1 | 28.7 | 39.5 | 48.3 | 62.1 | 27.2 |
| | Event 2K (T) | 40.1 | 50.5 | 61.6 | 29.5 | 41.2 | 46.3 | 62.1 | 26.1 |
| | T − C | −3 | 0 | −1.5 | 0.8 | 1.8 | −2 | 0.1 | −1.1 |
| | (T/C)% | 93 | 100 | 98 | 103 | 104 | 96 | 100 | 96 |
| | p-value | 0.13 | 0.98 | 0.22 | 0.52 | 0.28 | 0.25 | 0.97 | 0.52 |
| | Event 2I (T) | 41.2 | 49.6 | 61.2 | 29.5 | 39.9 | 46.7 | 64.1 | 25.4 |
| | T − C | −1.9 | −0.9 | −2 | 0.8 | 0.5 | −1.6 | 2 | −1.8 |
| | (T/C)% | 96 | 98 | 97 | 103 | 101 | 97 | 103 | 93 |
| | p-value | 0.42 | 0.56 | 0.13 | 0.56 | 0.76 | 0.23 | 0.27 | 0.35 |
| 3 | Control (C) | 40.3 | 49.9 | 61.1 | 28.8 | 39.7 | 47.6 | 64.6 | 25.9 |
| | Event 3O (T) | 42.5 | 45.5 | 60.4 | 29.6 | 39.4 | 48.7 | 63.2 | 28.6 |
| | T − C | 2.2 | −4.4 | −0.7 | 0.8 | −0.3 | 1.1 | −1.4 | 2.7 |
| | (T/C)% | 105 | 91 | 99 | 103 | 99 | 102 | 98 | 110 |
| | p-value | 0.28 | 0.01 | 0.68 | 0.69 | 0.83 | 0.35 | 0.44 | 0.04 |
| | Event 3N (T) | 39 | 50.9 | 63.2 | 30 | 40.4 | 49.4 | 64.9 | 28.8 |
| | T − C | −1.2 | 1 | 2.1 | 1.1 | 0.7 | 1.7 | 0.3 | 2.9 |
| | (T/C)% | 97 | 102 | 103 | 104 | 102 | 104 | 100 | 111 |
| | p-value | 0.44 | 0.46 | 0.07 | 0.43 | 0.68 | 0.13 | 0.86 | 0.02 |
| | Event 3L (T) | 38.2 | 51.7 | 61.1 | 31.8 | 39.6 | 50.5 | 64.1 | 28.8 |
| | T − C | −2.1 | 1.8 | −0.1 | 2.9 | −0.1 | 2.8 | −0.5 | 2.9 |
| | (T/C)% | 95 | 104 | 100 | 110 | 100 | 106 | 99 | 111 |
| | p-value | 0.21 | 0.19 | 0.96 | 0.03 | 0.93 | 0.03 | 0.77 | 0.02 |
| | Event 3Q (T) | 37.6 | 50.7 | 62.6 | 30.8 | 38.1 | 49.7 | 61.6 | 29.9 |
| | T − C | −2.7 | 0.8 | 1.5 | 2 | −1.6 | 2.1 | −3 | 4 |
| | (T/C)% | 93 | 102 | 102 | 107 | 96 | 104 | 95 | 115 |
| | p-value | 0.13 | 0.59 | 0.26 | 0.22 | 0.24 | 0.09 | 0.07 | 0 |
| | Event 3P (T) | 38.8 | 50.4 | 61.5 | 31.4 | 38.8 | 49.7 | 62.3 | 31.2 |
| | T − C | −1.5 | 0.5 | 0.4 | 2.6 | −0.9 | 2.1 | −2.3 | 5.3 |
| | (T/C)% | 96 | 101 | 101 | 109 | 98 | 104 | 96 | 120 |
| | p-value | 0.39 | 0.72 | 0.74 | 0.07 | 0.54 | 0.07 | 0.13 | 0 |
| | Event 3S (T) | 39.1 | 49 | 62.7 | 29.3 | 37.7 | 49.4 | 63.7 | 28.3 |
| | T − C | −1.2 | −0.9 | 1.6 | 0.5 | −2 | 1.8 | −0.9 | 2.4 |
| | (T/C)% | 97 | 98 | 103 | 102 | 95 | 104 | 99 | 109 |
| | p-value | 0.48 | 0.51 | 0.21 | 0.74 | 0.13 | 0.1 | 0.6 | 0.05 |
| | Event 3M (T) | 38.9 | 49.6 | 60.7 | 30.5 | 39 | 51.2 | 61.2 | 31 |
| | T − C | −1.4 | −0.3 | −0.4 | 1.7 | −0.7 | 3.6 | −3.4 | 5.1 |
| | (T/C)% | 97 | 99 | 99 | 106 | 98 | 108 | 95 | 120 |
| | p-value | 0.42 | 0.83 | 0.72 | 0.23 | 0.61 | 0 | 0.04 | 0 |
| | Event 3R (T) | 37.7 | 51.9 | 62.2 | 29.9 | 37.1 | 50.4 | 62.8 | 30.5 |
| | T − C | −2.6 | 2 | 1.1 | 1.1 | −2.6 | 2.8 | −1.8 | 4.6 |
| | (T/C)% | 94 | 104 | 102 | 104 | 93 | 106 | 97 | 118 |
| | p-value | 0.12 | 0.16 | 0.3 | 0.42 | 0.04 | 0.01 | 0.25 | 0 |

DM = dry matter, NDF = Neutral detergent fiber, NDFD30 = 30-h digestibility of NDF.

F1 hybrids from crosses of each transgenic event with one or two testers were grown in two field locations with six replications per location in a randomized complete block design. Six plants with ears removed were collected for analysis. Forage quality measurements were measured by standard NIRS (near-infrared spectroscopy). The test results are summarized in Tables 6B, 6C, 6D and 6E. Multiple Events of constructs 3, 4, 5 and 8 clearly showed significant increases in NDF digestibility (NDFD).

Several events showed increased NDFD30 and IVTDMD30 with decreased NDF and ADF. These results are consistent with expectations of corn silage as NDF and ADF decrease, digestibility is improved. Improved digestibility typically results in increased DM intake and fat-corrected milk production in cattle.

Crude Protein was also increased in several events, in contrast to traditional BMR hybrids which do not provide higher CP values (Oba and Allen, J. Dairy Sci., 1999, 82:135-142; Oba and Allen, J. Dairy Sci. 1999, 82:589-596). CP is also an important component of silage quality due to the requirement of the rumen microbes and the animal, and the contribution of CP to energy in the summative equation used by dairy nutritionists in evaluating energy of forages.

TABLE 6B
In vitro digestibility tests of plant samples without ears, measured by NIRS (Unit = % DM).

| | | Tester 2 (yellow dent) | | | | | | |
|---|---|---|---|---|---|---|---|---|
| | | DMYLD | MST | Prot | NDF | ADF | NDFD30 | IVTDMD30 |
| Construct | Description | (ton/a) | (% DM) | (% DM) | (% DM) | (% DM) | (% DM) | (% DM) |
| 1 | Event 1E (T) | 9.5 | 53.7 | 5.1 | 69.8 | 42.9 | 43.4 | 60.4 |
| | Control (C) | 9.9 | 54.6 | 4.7 | 66.5 | 41.5 | 44.7 | 63.2 |
| | T − C | −0.4 | −0.9 | 0.4 | 3.3 | 1.4 | −1.4 | −2.8 |
| | (T/C)% | 96 | 98 | 108 | 105 | 103 | 97 | 96 |
| | p-value | 0.25 | 0.61 | 0.58 | 0.03 | 0.16 | 0.17 | 0.02 |
| | Event 1H (T) | 8.8 | 54.3 | 6.0 | 60.7 | 36.6 | 45.7 | 67.0 |
| | Control (C) | 9.9 | 54.6 | 4.7 | 66.5 | 41.5 | 44.7 | 63.2 |
| | T − C | −1.1 | −0.3 | 1.3 | −5.8 | −4.9 | 1.0 | 3.8 |
| | (T/C)% | 89 | 99 | 128 | 91 | 88 | 102 | 106 |
| | p-value | 0.02 | 0.79 | 0.02 | 0.00 | 0.00 | 0.32 | 0.01 |
| 3 | Event 3N (T) | 8.8 | 56.5 | 5.1 | 66.8 | 41.1 | 49.0 | 65.9 |
| | Control (C) | 9.4 | 55.9 | 5.0 | 68.0 | 42.0 | 45.4 | 62.8 |
| | T − C | −0.5 | 0.6 | 0.1 | −1.2 | −0.9 | 3.6 | 3.1 |
| | (T/C)% | 94 | 101 | 103 | 98 | 98 | 108 | 105 |
| | p-value | 0.45 | 0.60 | 0.69 | 0.22 | 0.24 | 0.00 | 0.01 |
| | Event 3L (T) | 9.0 | 55.8 | 4.6 | 66.4 | 41.6 | 49.0 | 66.1 |
| | Control (C) | 9.4 | 55.9 | 5.0 | 68.0 | 42.0 | 45.4 | 62.8 |
| | T − C | −0.4 | 0.0 | −0.4 | −1.6 | −0.4 | 3.6 | 3.2 |
| | (T/C)% | 96 | 100 | 93 | 98 | 99 | 108 | 105 |
| | p-value | 0.91 | 0.62 | 0.24 | 0.11 | 0.56 | 0.01 | 0.01 |
| | Event 3Q (T) | 8.9 | 56.0 | 5.0 | 66.3 | 41.3 | 48.2 | 65.6 |
| | Control (C) | 9.4 | 55.9 | 5.0 | 68.0 | 42.0 | 45.4 | 62.8 |
| | T − C | −0.5 | 0.1 | 0.0 | −1.6 | −0.7 | 2.8 | 2.8 |
| | (T/C)% | 95 | 100 | 100 | 98 | 98 | 106 | 104 |
| | p-value | 0.73 | 0.91 | 0.97 | 0.11 | 0.33 | 0.01 | 0.01 |
| | Event 3P (T) | 9.6 | 56.7 | 5.3 | 65.9 | 40.7 | 50.8 | 67.6 |
| | Control (C) | 9.4 | 55.9 | 5.0 | 68.0 | 42.0 | 45.4 | 62.8 |
| | T − C | 0.2 | 0.8 | 0.3 | −2.0 | −1.3 | 5.4 | 4.8 |
| | (T/C)% | 102 | 101 | 106 | 97 | 97 | 112 | 108 |
| | p-value | 0.52 | 0.35 | 0.42 | 0.08 | 0.10 | 0.00 | 0.00 |

DM = dry matter, DMYLD = dry matter yield, Prot = protein, NDF = Neutral detergent fiber, ADF = acid detergent fiber, NDFD30 = 30-h digestibility of NDF, IVTDMD30 = 30-h digestibility of total dry matter in vitro.

TABLE 6C
In vitro digestibility tests of plant samples without ears, measured by NIRS (Unit = % DM).

| | | Tester 75 (yellow dent) | | | | | | |
|---|---|---|---|---|---|---|---|---|
| | | DMYLD | MST | Prot | NDF | ADF | NDFD30 | IVTDMD30 |
| Construct | Description | (ton/a) | (% DM) | (% DM) | (% DM) | (% DM) | (% DM) | (% DM) |
| 1 | Event 1E (T) | 9.8 | 56.5 | 5.5 | 69.4 | 43.2 | 39.4 | 57.9 |
| | Control (C) | 10.4 | 57.9 | 6.1 | 67.1 | 41.3 | 40.8 | 60.1 |
| | T − C | −0.6 | −1.4 | −0.6 | 2.3 | 1.9 | −1.3 | −2.2 |
| | (T/C)% | 94 | 98 | 90 | 103 | 105 | 97 | 96 |
| | p-value | 0.36 | 0.54 | 0.22 | 0.20 | 0.15 | 0.47 | 0.25 |
| | Event 1H (T) | 9.8 | 57.8 | 7.2 | 59.6 | 35.5 | 46.5 | 68.1 |
| | Control (C) | 10.4 | 57.9 | 6.1 | 67.1 | 41.3 | 40.8 | 60.1 |
| | T − C | −0.6 | 0.0 | 1.1 | −7.5 | −5.9 | 5.7 | 8.0 |
| | (T/C)% | 94 | 100 | 117 | 89 | 86 | 114 | 113 |
| | p-value | 0.40 | 0.97 | 0.05 | 0.00 | 0.00 | 0.00 | 0.00 |
| 3 | Event 3N (T) | 9.8 | 58.2 | 6.5 | 67.9 | 40.6 | 45.0 | 62.5 |
| | Control (C) | 10.5 | 56.2 | 6.1 | 66.8 | 40.5 | 42.3 | 61.3 |
| | T − C | −0.7 | 2.0 | 0.4 | 1.1 | 0.1 | 2.7 | 1.2 |
| | (T/C)% | 93 | 104 | 107 | 102 | 100 | 106 | 102 |
| | p-value | 0.29 | 0.11 | 0.45 | 0.48 | 0.95 | 0.02 | 0.38 |
| | Event 3L (T) | 9.9 | 58.1 | 6.2 | 70.3 | 42.9 | 42.0 | 59.1 |
| | Control (C) | 10.5 | 56.2 | 6.1 | 66.8 | 40.5 | 42.3 | 61.3 |
| | T − C | −0.7 | 1.9 | 0.1 | 3.5 | 2.4 | −0.3 | −2.2 |
| | (T/C)% | 94 | 103 | 102 | 105 | 106 | 99 | 96 |
| | p-value | 0.38 | 0.24 | 0.75 | 0.02 | 0.03 | 0.79 | 0.14 |
| | Event 3Q (T) | 10.3 | 56.8 | 6.6 | 67.9 | 40.9 | 43.6 | 61.6 |
| | Control (C) | 10.5 | 56.2 | 6.1 | 66.8 | 40.5 | 42.3 | 61.3 |
| | T − C | −0.2 | 0.6 | 0.5 | 1.1 | 0.4 | 1.3 | 0.3 |
| | (T/C)% | 98 | 101 | 109 | 102 | 101 | 103 | 101 |
| | p-value | 0.78 | 0.66 | 0.37 | 0.50 | 0.71 | 0.36 | 0.82 |
| | Event 3P (T) | 10.4 | 55.0 | 6.1 | 64.7 | 39.1 | 45.0 | 64.4 |
| | Control (C) | 10.5 | 56.2 | 6.1 | 66.8 | 40.5 | 42.3 | 61.3 |
| | T − C | −0.2 | −1.2 | 0.1 | −2.2 | −1.4 | 2.7 | 3.1 |
| | (T/C)% | 99 | 98 | 101 | 97 | 96 | 106 | 105 |
| | p-value | 0.75 | 0.37 | 0.84 | 0.13 | 0.14 | 0.01 | 0.02 |

DM = dry matter, DMYLD = dry matter yield, Prot = protein, NDF = Neutral detergent fiber, ADF = acid detergent fiber, NDFD30 = 30-h digestibility of NDF, IVTDMD30 = 30-h digestibility of total dry matter in vitro.

TABLE 6D
In vitro digestibility tests of plant samples without ears, measured by NIRS (Unit = % DM).

| | | Tester 1 (nutritionally enhanced) | | | | | | |
|---|---|---|---|---|---|---|---|---|
| | | DMYLD | MST | Prot | NDF | ADF | NDFD30 | IVTDMD30 |
| Construct | Description | (ton/a) | (% DM) | (% DM) | (% DM) | (% DM) | (% DM) | (% DM) |
| 4 | Event 4T (T) | 8.8 | 54.5 | 5.7 | 66.6 | 40.1 | 51.2 | 67.5 |
| | Control (C) | 9.1 | 56.2 | 6.5 | 69.3 | 41.6 | 45.0 | 61.8 |
| | T − C | −0.31 | −1.66 | −0.79 | −2.69 | −1.50 | 6.22 | 5.70 |
| | (T/C)% | 97 | 97 | 88 | 96 | 96 | 114 | 109 |
| | p-value | 0.92 | 0.30 | 0.64 | 0.41 | 0.41 | 0.00 | 0.00 |
| | Event 4Y (T) | 8.5 | 55.7 | 6.8 | 66.6 | 39.5 | 53.1 | 68.7 |
| | Control (C) | 9.1 | 56.2 | 6.5 | 69.3 | 41.6 | 45.0 | 61.8 |
| | T − C | −0.61 | −0.46 | 0.35 | −2.69 | −2.10 | 8.12 | 6.90 |
| | (T/C)% | 93 | 99 | 105 | 96 | 95 | 118 | 111 |
| | p-value | 0.63 | 0.92 | 0.50 | 0.16 | 0.08 | 0.00 | 0.00 |
| | Event 4V (T) | 8.1 | 54.5 | 7.2 | 67.6 | 39.7 | 53.6 | 68.7 |
| | Control (C) | 9.1 | 56.2 | 6.5 | 69.3 | 41.6 | 45.0 | 61.8 |
| | T − C | −1.01 | −1.66 | 0.75 | −1.69 | −1.90 | 8.62 | 6.90 |
| | (T/C)% | 89 | 97 | 112 | 98 | 95 | 119 | 111 |
| | p-value | 0.44 | 0.37 | 0.10 | 0.54 | 0.18 | 0.00 | 0.00 |
| | Event 4W (T) | 8.7 | 57.1 | 6.0 | 69.5 | 41.8 | 45.3 | 62.0 |
| | Control (C) | 9.1 | 56.2 | 6.5 | 69.3 | 41.6 | 45.0 | 61.8 |
| | T − C | −0.41 | 0.94 | −0.45 | 0.21 | 0.20 | 0.32 | 0.20 |
| | (T/C)% | 95 | 102 | 93 | 100 | 100 | 101 | 100 |
| | p-value | 0.92 | 0.97 | 0.88 | 0.72 | 0.76 | 0.58 | 0.96 |
| | Event 4X (T) | 8.2 | 57.9 | 5.4 | 67.9 | 41.2 | 47.1 | 64.1 |
| | Control (C) | 9.1 | 56.2 | 6.5 | 69.3 | 41.6 | 45.0 | 61.8 |
| | T − C | −0.91 | 1.74 | −1.05 | −1.39 | −0.40 | 2.12 | 2.30 |
| | (T/C)% | 90 | 103 | 84 | 98 | 99 | 105 | 104 |
| | p-value | 0.65 | 0.84 | 0.39 | 0.83 | 0.99 | 0.06 | 0.27 |
| 5 | Event 5Z (T) | 8.4 | 57.0 | 7.0 | 68.4 | 40.7 | 46.9 | 63.6 |
| | Control (C) | 9.2 | 57.9 | 5.7 | 69.6 | 42.0 | 45.3 | 61.9 |
| | T − C | −0.80 | −0.90 | 1.30 | −1.20 | −1.30 | 1.60 | 1.70 |
| | (T/C)% | 91 | 98 | 123 | 98 | 97 | 104 | 103 |
| | p-value | 0.23 | 0.71 | 0.02 | 0.39 | 0.08 | 0.13 | 0.14 |
| | Event 5B1 (T) | 9.4 | 57.0 | 6.4 | 69.1 | 41.3 | 48.6 | 64.5 |
| | Control (C) | 9.2 | 57.9 | 5.7 | 69.6 | 42.0 | 45.3 | 61.9 |
| | T − C | 0.20 | −0.90 | 0.70 | −0.50 | −0.70 | 3.30 | 2.60 |
| | (T/C)% | 102 | 98 | 112 | 99 | 98 | 107 | 104 |
| | p-value | 0.66 | 0.70 | 0.12 | 0.72 | 0.38 | 0.00 | 0.02 |
| | Event 5F1 (T) | 9.3 | 57.0 | 6.6 | 68.4 | 40.6 | 48.8 | 64.9 |
| | Control (C) | 9.2 | 57.9 | 5.7 | 69.6 | 42.0 | 45.3 | 61.9 |
| | T − C | 0.10 | −0.90 | 0.90 | −1.20 | −1.40 | 3.50 | 3.00 |
| | (T/C)% | 101 | 98 | 116 | 98 | 97 | 108 | 105 |
| | p-value | 0.63 | 0.62 | 0.04 | 0.34 | 0.07 | 0.00 | 0.02 |
| 6 | Event 6G1 (T) | 9.3 | 55.2 | 6.5 | 69.8 | 41.6 | 45.9 | 62.2 |
| | Control (C) | 10.1 | 56.2 | 6.1 | 70.0 | 42.0 | 45.1 | 61.5 |
| | T − C | −0.80 | −1.00 | 0.40 | −0.20 | −0.40 | 0.80 | 0.70 |
| | (T/C)% | 92 | 98 | 107 | 100 | 99 | 102 | 101 |
| | p-value | 0.13 | 0.64 | 0.37 | 0.85 | 0.64 | 0.43 | 0.56 |
| | Event 6H1 (T) | 9.7 | 55.3 | 6.6 | 68.9 | 41.0 | 46.3 | 62.9 |
| | Control (C) | 10.1 | 56.2 | 6.1 | 70.0 | 42.0 | 45.1 | 61.5 |
| | T − C | −0.40 | −0.90 | 0.50 | −1.10 | −1.00 | 1.20 | 1.40 |
| | (T/C)% | 96 | 98 | 108 | 98 | 98 | 103 | 102 |
| | p-value | 0.52 | 0.51 | 0.20 | 0.22 | 0.12 | 0.18 | 0.13 |
| | Event 6K1 (T) | 9.5 | 57.2 | 6.6 | 70.7 | 42.3 | 44.7 | 60.9 |
| | Control (C) | 10.1 | 56.2 | 6.1 | 70.0 | 42.0 | 45.1 | 61.5 |
| | T − C | −0.60 | 1.00 | 0.50 | 0.70 | 0.30 | −0.40 | −0.60 |
| | (T/C)% | 94 | 102 | 108 | 101 | 101 | 99 | 99 |
| | p-value | 0.25 | 0.36 | 0.29 | 0.53 | 0.72 | 0.65 | 0.53 |
| | Event 6L1 (T) | 9.7 | 55.5 | 6.0 | 71.7 | 42.9 | 44.4 | 60.0 |
| | Control (C) | 10.1 | 56.2 | 6.1 | 70.0 | 42.0 | 45.1 | 61.5 |
| | T − C | −0.40 | −0.70 | −0.10 | 1.70 | 0.90 | −0.70 | −1.50 |
| | (T/C)% | 96 | 99 | 98 | 102 | 102 | 98 | 98 |
| | p-value | 0.52 | 0.56 | 0.76 | 0.06 | 0.18 | 0.37 | 0.11 |
| | Event 6N1 (T) | 10.0 | 55.1 | 6.4 | 69.4 | 41.4 | 45.8 | 62.3 |
| | Control (C) | 10.1 | 56.2 | 6.1 | 70.0 | 42.0 | 45.1 | 61.5 |
| | T − C | −0.1 | −1.1 | 0.3 | −0.6 | −0.6 | 0.7 | 0.8 |
| | (T/C)% | 99 | 98 | 105 | 99 | 99 | 102 | 101 |
| | p-value | 0.38 | 0.28 | 0.36 | 0.42 | 0.32 | 0.43 | 0.35 |
| | Event 6O1 (T) | 9.9 | 57.7 | 6.5 | 72.2 | 43.0 | 45.2 | 60.4 |
| | Control (C) | 10.1 | 56.2 | 6.1 | 70.0 | 42.0 | 45.1 | 61.5 |
| | T − C | −0.2 | 1.5 | 0.4 | 2.2 | 1.0 | 0.1 | −1.1 |
| | (T/C)% | 98 | 103 | 107 | 103 | 102 | 100 | 98 |
| | p-value | 0.78 | 0.18 | 0.28 | 0.02 | 0.10 | 0.95 | 0.21 |
| | Event 6Q1 (T) | 9.7 | 55.6 | 6.6 | 70.5 | 42.3 | 45.4 | 61.4 |
| | Control (C) | 10.1 | 56.2 | 6.1 | 70.0 | 42.0 | 45.1 | 61.5 |
| | T − C | −0.4 | −0.6 | 0.5 | 0.5 | 0.3 | 0.3 | −0.1 |
| | (T/C)% | 96 | 99 | 108 | 101 | 101 | 101 | 100 |
| | p-value | 0.51 | 0.72 | 0.27 | 0.58 | 0.69 | 0.80 | 0.93 |
| 7 | Event 7T1 (T) | 9.5 | 59.3 | 6.2 | 70.1 | 42.0 | 45.3 | 61.6 |
| | Control (C) | 9.8 | 57.4 | 6.1 | 69.6 | 41.6 | 45.7 | 62.1 |
| | T − C | −0.3 | 1.9 | 0.1 | 0.5 | 0.4 | −0.4 | −0.5 |
| | (T/C)% | 97 | 103 | 102 | 101 | 101 | 99 | 99 |
| | p-value | 0.60 | 0.30 | 0.73 | 0.59 | 0.62 | 0.57 | 0.57 |
| | Event 7W1 (T) | 9.8 | 58.7 | 6.2 | 69.3 | 41.6 | 45.1 | 61.9 |
| | Control (C) | 9.8 | 57.4 | 6.1 | 69.6 | 41.6 | 45.7 | 62.1 |
| | T − C | 0.0 | 1.3 | 0.1 | −0.3 | 0.0 | −0.6 | −0.2 |
| | (T/C)% | 100 | 102 | 102 | 100 | 100 | 99 | 100 |
| | p-value | 0.96 | 0.43 | 0.81 | 0.83 | 0.91 | 0.47 | 0.73 |
| | Event 7Y1 (T) | 9.0 | 55.6 | 6.2 | 68.5 | 40.7 | 50.6 | 66.1 |
| | Control (C) | 9.8 | 57.4 | 6.1 | 69.6 | 41.6 | 45.7 | 62.1 |
| | T − C | −0.8 | −1.8 | 0.1 | −1.1 | −0.9 | 4.9 | 4.0 |
| | (T/C)% | 92 | 97 | 102 | 98 | 98 | 111 | 106 |
| | p-value | 0.49 | 0.19 | 0.54 | 0.42 | 0.28 | 0.00 | 0.00 |
| | Event 7Z1 (T) | 8.7 | 57.3 | 6.2 | 69.7 | 41.5 | 48.9 | 64.3 |
| | Control (C) | 9.8 | 57.4 | 6.1 | 69.6 | 41.6 | 45.7 | 62.1 |
| | T − C | −1.1 | −0.1 | 0.1 | 0.1 | −0.1 | 3.2 | 2.2 |
| | (T/C)% | 89 | 100 | 102 | 100 | 100 | 107 | 104 |
| | p-value | 0.20 | 0.89 | 0.62 | 0.99 | 0.78 | 0.01 | 0.07 |
| 8 | Event 8C2 (T) | 9.5 | 53.1 | 8.6 | 66.3 | 38.3 | 54.5 | 69.8 |
| | Control (C) | 9.4 | 55.6 | 6.7 | 68.6 | 41.1 | 46.0 | 62.9 |
| | T − C | 0.1 | −2.5 | 1.9 | −2.3 | −2.8 | 8.5 | 6.9 |
| | (T/C)% | 101 | 96 | 128 | 97 | 93 | 118 | 111 |
| | p-value | 0.98 | 0.29 | 0.00 | 0.13 | 0.01 | 0.00 | 0.00 |
| | Event 8D2 (T) | 10.3 | 52.2 | 7.4 | 66.5 | 39.4 | 54.9 | 70.0 |
| | Control (C) | 9.4 | 55.6 | 6.7 | 68.6 | 41.1 | 46.0 | 62.9 |
| | T − C | 0.9 | −3.4 | 0.7 | −2.1 | −1.7 | 8.9 | 7.1 |
| | (T/C)% | 110 | 94 | 110 | 97 | 96 | 119 | 111 |
| | p-value | 0.04 | 0.07 | 0.06 | 0.07 | 0.03 | 0.00 | 0.00 |
| | Event 8E2 (T) | 9.4 | 54.3 | 7.3 | 66.8 | 39.4 | 52.9 | 68.5 |
| | Control (C) | 9.4 | 55.6 | 6.7 | 68.6 | 41.1 | 46.0 | 62.9 |
| | T − C | 0.0 | −1.3 | 0.6 | −1.8 | −1.7 | 6.9 | 5.6 |
| | (T/C)% | 100 | 98 | 109 | 97 | 96 | 115 | 109 |
| | p-value | 0.94 | 0.61 | 0.08 | 0.18 | 0.07 | 0.00 | 0.00 |
| | Event 8F2 (T) | 9.5 | 56.1 | 7.6 | 66.7 | 39.3 | 53.6 | 69.1 |
| | Control (C) | 9.4 | 55.6 | 6.7 | 68.6 | 41.1 | 46.0 | 62.9 |
| | T − C | 0.1 | 0.5 | 0.9 | −1.9 | −1.8 | 7.6 | 6.2 |
| | (T/C)% | 101 | 101 | 113 | 97 | 96 | 117 | 110 |
| | p-value | 0.74 | 0.72 | 0.01 | 0.10 | 0.02 | 0.00 | 0.00 |
| | Event 8G2 (T) | 9.4 | 53.7 | 7.5 | 67.7 | 40.0 | 54.5 | 69.2 |
| | Control (C) | 9.4 | 55.6 | 6.7 | 68.6 | 41.1 | 46.0 | 62.9 |
| | T − C | 0.0 | −1.9 | 0.8 | −0.9 | −1.1 | 8.5 | 6.3 |
| | (T/C)% | 100 | 97 | 112 | 99 | 97 | 118 | 110 |
| | p-value | 0.93 | 0.35 | 0.09 | 0.47 | 0.20 | 0.00 | 0.00 |
| | Event 8H2 (T) | 8.9 | 56.0 | 7.1 | 67.9 | 40.1 | 53.8 | 68.6 |
| | Control (C) | 9.4 | 55.6 | 6.7 | 68.6 | 41.1 | 46.0 | 62.9 |
| | T − C | −0.5 | 0.4 | 0.4 | −0.7 | −1.0 | 7.8 | 5.7 |
| | (T/C)% | 95 | 101 | 106 | 99 | 98 | 117 | 109 |
| | p-value | 0.30 | 0.78 | 0.29 | 0.52 | 0.20 | 0.00 | 0.00 |
| | Event 8J2 (T) | 10.4 | 53.8 | 7.0 | 70.4 | 41.7 | 46.5 | 62.4 |
| | Control (C) | 9.4 | 55.6 | 6.7 | 68.6 | 41.1 | 46.0 | 62.9 |
| | T − C | 1.0 | −1.8 | 0.3 | 1.8 | 0.6 | 0.5 | −0.5 |
| | (T/C)% | 111 | 97 | 104 | 103 | 101 | 101 | 99 |
| | p-value | 0.03 | 0.38 | 0.39 | 0.14 | 0.45 | 0.62 | 0.64 |
| 10 | Event 10O2 (T) | 8.6 | 57.9 | 6.5 | 67.1 | 40.9 | 42.3 | 61.7 |
| | Control (C) | 9.5 | 55.6 | 6.5 | 66.8 | 40.3 | 44.4 | 62.9 |
| | T − C | −0.9 | 2.3 | 0.0 | 0.3 | 0.6 | −2.1 | −1.2 |
| | (T/C)% | 91 | 104 | 100 | 100 | 101 | 95 | 98 |
| | p-value | 0.25 | 0.27 | 0.78 | 0.67 | 0.52 | 0.16 | 0.27 |
| | Event 10P2 (T) | 9.2 | 57.3 | 6.4 | 63.3 | 38.2 | 45.3 | 65.3 |
| | Control (C) | 9.5 | 55.6 | 6.5 | 66.8 | 40.3 | 44.4 | 62.9 |
| | T − C | −0.3 | 1.7 | −0.1 | −3.5 | −2.1 | 0.9 | 2.4 |
| | (T/C)% | 97 | 103 | 98 | 95 | 95 | 102 | 104 |
| | p-value | 0.83 | 0.47 | 0.96 | 0.02 | 0.05 | 0.37 | 0.05 |
| | Event 10T2 (T) | 10.5 | 53.7 | 7.0 | 66.0 | 39.6 | 44.1 | 63.1 |
| | Control (C) | 9.5 | 55.6 | 6.5 | 66.8 | 40.3 | 44.4 | 62.9 |
| | T − C | 1.0 | −1.9 | 0.5 | −0.8 | −0.7 | −0.3 | 0.2 |
| | (T/C)% | 111 | 97 | 108 | 99 | 98 | 99 | 100 |
| | p-value | 0.22 | 0.30 | 0.26 | 0.56 | 0.45 | 0.72 | 0.87 |
| | Event 10U2 (T) | 7.6 | 55.8 | 7.9 | 64.2 | 38.0 | 46.5 | 65.7 |
| | Control (C) | 9.5 | 55.6 | 6.5 | 66.8 | 40.3 | 44.4 | 62.9 |
| | T − C | −1.9 | 0.2 | 1.4 | −2.6 | −2.3 | 2.1 | 2.8 |
| | (T/C)% | 80 | 100 | 122 | 96 | 94 | 105 | 104 |
| | p-value | 0.02 | 0.93 | 0.00 | 0.06 | 0.02 | 0.04 | 0.02 |

DM = dry matter, DMYLD = dry matter yield, Prot = protein, NDF = Neutral detergent fiber, ADF = acid detergent fiber, NDFD30 = 30-h digestibility of NDF, IVTDMD30 = 30-h digestibility of total dry matter in vitro.

TABLE 6E
In vitro digestibility tests of plant samples without ears, measured by NIRS (Unit = % DM).

| | | Tester 2 (yellow dent) | | | | | | |
|---|---|---|---|---|---|---|---|---|
| | | DMYLD | MST | Prot | NDF | ADF | NDFD30 | IVTDMD30 |
| Construct | Description | (ton/a) | (% DM) | (% DM) | (% DM) | (% DM) | (% DM) | (% DM) |
| 4 | Event 4W (T) | 8.8 | 58.7 | 5.3 | 58.1 | 35.5 | 58.2 | 75.5 |
| | Control (C) | 8.5 | 59.4 | 4.5 | 65.6 | 40.9 | 48.3 | 66.0 |
| | T − C | 0.30 | −0.70 | 0.80 | −7.50 | −5.40 | 9.90 | 9.50 |
| | (T/C)% | 104 | 99 | 118 | 89 | 87 | 120 | 114 |
| | p-value | 0.60 | 0.64 | 0.05 | 0.00 | 0.00 | 0.00 | 0.00 |
| 5 | Event 5B1 (T) | 8.6 | 58.6 | 5.8 | 55.6 | 33.9 | 56.8 | 76.0 |
| | Control (C) | 7.5 | 58.6 | 4.1 | 64.5 | 40.6 | 45.7 | 64.9 |
| | T − C | 1.10 | 0.00 | 1.70 | −8.90 | −6.70 | 11.10 | 11.10 |
| | (T/C)% | 115 | 100 | 141 | 86 | 83 | 124 | 117 |
| | p-value | 0.29 | 0.51 | 0.00 | 0.00 | 0.01 | 0.00 | 0.00 |
| | Event 5E1 (T) | 7.6 | 61.3 | 4.5 | 62.9 | 39.2 | 50.0 | 68.5 |
| | Control (C) | 7.5 | 58.6 | 4.1 | 64.5 | 40.6 | 45.7 | 64.9 |
| | T − C | 0.10 | 2.70 | 0.40 | −1.60 | −1.40 | 4.30 | 3.60 |
| | (T/C)% | 101 | 105 | 110 | 98 | 97 | 109 | 106 |
| | p-value | 0.76 | 0.08 | 0.64 | 0.19 | 0.15 | 0.01 | 0.01 |
| | Event 5A4 (T) | 8.2 | 57.9 | 5.4 | 62.4 | 38.6 | 50.9 | 69.3 |
| | Control (C) | 7.5 | 58.6 | 4.1 | 64.5 | 40.6 | 45.7 | 64.9 |
| | T − C | 0.70 | −0.70 | 1.30 | −2.10 | −2.00 | 5.20 | 4.40 |
| | (T/C)% | 109 | 99 | 132 | 97 | 95 | 111 | 107 |
| | p-value | 0.37 | 0.87 | 0.01 | 0.12 | 0.05 | 0.00 | 0.00 |
| 6 | Event 6H1 (T) | 9.8 | 52.3 | 4.9 | 66.6 | 41.0 | 45.4 | 63.5 |
| | Control (C) | 9.7 | 55.5 | 4.5 | 66.1 | 41.2 | 43.5 | 62.5 |
| | T − C | 0.10 | −3.20 | 0.40 | 0.50 | −0.20 | 1.90 | 1.00 |
| | (T/C)% | 101 | 94 | 109 | 101 | 100 | 104 | 102 |
| | p-value | 0.99 | 0.13 | 0.43 | 0.55 | 0.91 | 0.13 | 0.50 |
| | Event 6K1 (T) | 9.5 | 54.0 | 4.9 | 63.7 | 38.8 | 45.7 | 64.8 |
| | Control (C) | 9.7 | 55.5 | 4.5 | 66.1 | 41.2 | 43.5 | 62.5 |
| | T − C | −0.20 | −1.50 | 0.40 | −2.40 | −2.40 | 2.20 | 2.30 |
| | (T/C)% | 98 | 97 | 109 | 96 | 94 | 105 | 104 |
| | p-value | 0.73 | 0.44 | 0.41 | 0.27 | 0.18 | 0.12 | 0.20 |
| | Event 6N1 (T) | 8.6 | 56.9 | 4.7 | 66.1 | 40.7 | 45.4 | 63.7 |
| | Control (C) | 9.7 | 55.5 | 4.5 | 66.1 | 41.2 | 43.5 | 62.5 |
| | T − C | −1.1 | 1.4 | 0.2 | 0.0 | −0.5 | 1.9 | 1.2 |
| | (T/C)% | 89 | 103 | 104 | 100 | 99 | 104 | 102 |
| | p-value | 0.24 | 0.53 | 0.36 | 0.54 | 0.23 | 0.04 | 0.12 |
| | Event 6O1 (T) | 9.1 | 54.0 | 4.8 | 63.7 | 39.0 | 45.5 | 64.9 |
| | Control (C) | 9.7 | 55.5 | 4.5 | 66.1 | 41.2 | 43.5 | 62.5 |
| | T − C | −0.6 | −1.5 | 0.3 | −2.4 | −2.2 | 2.0 | 2.4 |
| | (T/C)% | 94 | 97 | 107 | 96 | 95 | 105 | 104 |
| | p-value | 0.42 | 0.48 | 0.49 | 0.11 | 0.09 | 0.14 | 0.10 |
| | Event 6Q1 (T) | 5.8 | 47.1 | 5.0 | 61.9 | 38.3 | 45.6 | 66.1 |
| | Control (C) | 9.7 | 55.5 | 4.5 | 66.1 | 41.2 | 43.5 | 62.5 |
| | T − C | −3.9 | −8.4 | 0.5 | −4.2 | −2.9 | 2.1 | 3.6 |
| | (T/C)% | 60 | 85 | 111 | 94 | 93 | 105 | 106 |
| | p-value | 0.00 | 0.22 | 0.39 | 0.00 | 0.01 | 0.19 | 0.02 |
| 7 | Event 7S1 (T) | 8.6 | 55.7 | 5.1 | 64.6 | 39.7 | 44.8 | 64.2 |
| | Control (C) | 8.3 | 55.1 | 5.4 | 65.8 | 40.4 | 43.6 | 62.7 |
| | T − C | 0.3 | 0.6 | −0.3 | −1.2 | −0.7 | 1.2 | 1.5 |
| | (T/C)% | 104 | 101 | 94 | 98 | 98 | 103 | 102 |
| | p-value | 0.55 | 0.64 | 0.51 | 0.14 | 0.26 | 0.18 | 0.11 |
| | Event 7T1 (T) | 8.5 | 53.4 | 5.0 | 67.2 | 41.1 | 45.2 | 63.0 |
| | Control (C) | 8.3 | 55.1 | 5.4 | 65.8 | 40.4 | 43.6 | 62.7 |
| | T − C | 0.2 | −1.7 | −0.4 | 1.4 | 0.7 | 1.6 | 0.3 |
| | (T/C)% | 102 | 97 | 93 | 102 | 102 | 104 | 100 |
| | p-value | 0.68 | 0.25 | 0.31 | 0.17 | 0.31 | 0.09 | 0.73 |
| | Event 7W1 (T) | 9.1 | 55.6 | 4.8 | 66.6 | 41.4 | 43.0 | 61.9 |
| | Control (C) | 8.3 | 55.1 | 5.4 | 65.8 | 40.4 | 43.6 | 62.7 |
| | T − C | 0.8 | 0.5 | −0.6 | 0.8 | 1.0 | −0.6 | −0.8 |
| | (T/C)% | 110 | 101 | 89 | 101 | 102 | 99 | 99 |
| | p-value | 0.13 | 0.79 | 0.24 | 0.59 | 0.27 | 0.79 | 0.65 |
| | Event 7Y1 (T) | 8.3 | 55.5 | 5.6 | 68.8 | 41.5 | 44.9 | 62.1 |
| | Control (C) | 8.3 | 55.1 | 5.4 | 65.8 | 40.4 | 43.6 | 62.7 |
| | T − C | 0.0 | 0.4 | 0.2 | 3.0 | 1.1 | 1.3 | −0.6 |
| | (T/C)% | 100 | 101 | 104 | 105 | 103 | 103 | 99 |
| | p-value | 0.90 | 0.81 | 0.66 | 0.02 | 0.14 | 0.14 | 0.48 |
| | Event 7Z1 (T) | 9.3 | 52.4 | 4.9 | 66.6 | 41.0 | 45.1 | 63.4 |
| | Control (C) | 8.3 | 55.1 | 5.4 | 65.8 | 40.4 | 43.6 | 62.7 |
| | T − C | 1.0 | −2.7 | −0.5 | 0.8 | 0.6 | 1.5 | 0.7 |
| | (T/C)% | 112 | 95 | 91 | 101 | 101 | 103 | 101 |
| | p-value | 0.08 | 0.05 | 0.25 | 0.47 | 0.35 | 0.10 | 0.48 |
| 10 | Event 10O2 (T) | 9.7 | 54.7 | 5.9 | 69.0 | 41.3 | 45.2 | 62.1 |
| | Control (C) | 8.6 | 55.7 | 5.1 | 67.6 | 40.9 | 44.2 | 62.1 |
| | T − C | 1.1 | −1.0 | 0.8 | 1.4 | 0.4 | 1.0 | 0.0 |
| | (T/C)% | 113 | 98 | 116 | 102 | 101 | 102 | 100 |
| | p-value | 0.11 | 0.67 | 0.18 | 0.48 | 0.72 | 0.59 | 0.91 |
| | Event 10P2 (T) | 9.3 | 55.3 | 4.9 | 68.7 | 41.8 | 45.9 | 62.7 |
| | Control (C) | 8.6 | 55.7 | 5.1 | 67.6 | 40.9 | 44.2 | 62.1 |
| | T − C | 0.7 | −0.4 | −0.2 | 1.1 | 0.9 | 1.7 | 0.6 |
| | (T/C)% | 108 | 99 | 96 | 102 | 102 | 104 | 101 |
| | p-value | 0.19 | 0.84 | 0.67 | 0.56 | 0.51 | 0.34 | 0.77 |
| | Event 10Q2 (T) | 9.6 | 53.5 | 5.1 | 67.5 | 41.5 | 44.7 | 62.6 |
| | Control (C) | 8.6 | 55.7 | 5.1 | 67.6 | 40.9 | 44.2 | 62.1 |
| | T − C | 1.0 | −2.2 | 0.0 | −0.1 | 0.6 | 0.5 | 0.5 |
| | (T/C)% | 112 | 96 | 100 | 100 | 101 | 101 | 101 |
| | p-value | 0.15 | 0.06 | 0.52 | 0.95 | 0.59 | 0.99 | 0.96 |
| | Event 10U2 (T) | 8.7 | 54.3 | 5.6 | 65.6 | 39.4 | 46.0 | 64.3 |
| | Control (C) | 8.6 | 55.7 | 5.1 | 67.6 | 40.9 | 44.2 | 62.1 |
| | T − C | 0.1 | −1.4 | 0.5 | −2.0 | −1.5 | 1.8 | 2.2 |
| | (T/C)% | 101 | 97 | 110 | 97 | 96 | 104 | 104 |
| | p-value | 0.89 | 0.25 | 0.37 | 0.39 | 0.43 | 0.26 | 0.23 |

DM = dry matter, DMYLD = dry matter yield, Prot = protein, NDF = Neutral detergent fiber, ADF = acid detergent fiber, NDFD30 = 30-h digestibility of NDF, IVTDMD30 = 30-h digestibility of total dry matter in vitro.

### Example 9

### Cloning of a Promoter Region of Maize Caffeic Acid O-methyltransferase

To identify additional promoters for transgene expression, a promoter region of ZmCOMT was amplified by PCR from genomic DNA of maize line W64A. A DNA sequence including the coding region (CDS) and a partial 5′ untranslated region (5′UTR) of the COMT gene was obtained from the NCBI Gen Bank database (Accession No. M73235) based on published literature (Capellades et al., Plant Mol. Biol., 1996, 31:307-322). The sequence of the reverse PCR primer for ZmCOMT promoter cloning was based on the sequence of Accession No. M73235.

Although Capellades et al. described the cloning of the COMT promoter and characterization of its expression pattern, the authors did not disclose the promoter DNA sequence. Similarly, Accession No. M73235 contains only the coding sequence plus 130 nucleotides of 5′UTR. Therefore, to obtain additional sequence for design of the forward primer for ZmCOMT promoter cloning, a BLAST search was performed using the M73235 sequence as query against a maize genomic database. A sequence with homology to M73235 was identified and a forward PCR primer was designed based on this sequence. The forward and reverse primer sequences for PCR cloning of the ZmCOMT promoter are shown below:

(SEQ ID NO: 41)
Forward primer: GCCTTAATTAAggagatgatctcaggctatc
(SEQ ID NO: 42)
Reverse primer: TAAGGCGCGCCAtcgaggagcgcgctagctg

PCR was performed at the following thermocycle conditions using KOD DNA polymerase: 94° C. for 2 min, then 30 cycles of 94° C. for 20 s, 58° C. for 10 s, and 68° C. for 1 min.

PCR products were purified and cloned into a TOPO-TA entry vector, PCR4—TOPOTA cloning vector (Invitrogen). The resulting vector was designated RHF180 and was sequenced for confirmation of the ZmCOMT promoter.

The ZmCOMT promoter sequence aligned with a promoter sequence of W64A COMT genomic DNA (Genbank Accession No. AY323283) including the 5′UTR is shown in FIG. 2A-C. The alignment shows that a gap and other polymorphisms exist between the two sequences.

### Example 10

### Construction of a Transgenic Expression Cassette and Super-Binary Vector Containing the ZmCOMT Promoter

To clone the ZmCOMT promoter fragment into a binary vector for plant transformation such as corn, another pair of primers with a PacI restriction enzyme site in the forward primer and an Ascl site in the reverse primer was synthesized. Their sequences are shown below:

(SEQ ID NO: 43)
Forward primer: GCCTTAATTAAggagatgatctcaggctatc
(SEQ ID NO: 44)
Reverse primer: TAAGGCGCGCCAtcgaggagcgcgctagctg

PCR was performed using these primers and RHF 180 DNA as template and the same thermocycle conditions as described above. PCR products were digested with PacI and AscI. The digested products were purified and ligated into PacI and AscI digested vector RTP1206 to generate a binary vector that contains a pZm-COMT::GUS::t-NOS cassette. The resulting vector was designated construct 11. Construct 11 was transformed into maize as described in Example 3 above.

### Example 11

### Characterization of ZmCOMT Promoter by GUS Expression Assay

ZmCOMT promoter expression patterns were measured using GUS histochemical analysis following published protocols (Jefferson, Plant Mol. Bio. Rep., 1987, 5:387-405; Jefferson et al., EMBO J., 1987, 6:3901-3907). Ten TO plants and five T1 plants with a single copy of the transgene were chosen for promoter analysis. GUS expression was measured in different tissues at various developmental stages:

TABLE 7
Tissues for measurement of ZmCOMT: GUS expression.

| | |
|---|---|
| Dev. | |
| Stage | Tissue |

| | | | |
|---|---|---|---|
| V5 | Stem (I3) | Leaf and midrib | Root |
| | | (3^{rd} leaf) | |
| V10 | Stem (I5, I10) | Leaf and midrib | Root |
| | | (5^{th} and 10^{th}) | |
| V15 | Stem (I5, I8, I13) | Leaf and midrib | Root |
| | | (5^{th}, 8^{th} and 13^{th}) | |

No GUS gene expression was detected in plants containing the transgene. This lack of detectable GUS expression indicates that the strength of the cloned ZmCOMT promoter is not sufficient to drive detectable levels of GUS expression in root, stem and leaf tissues of T0 and T1 transgenic plants.

### Example 12

### Characterization of the ZmCOMT Promoter by Transgene Expression and a Transgene Target Assay

The ZmCOMT promoter was tested with a trait gene of interest by making construct 1, that contains the transgene cassette of p-ZmCOMT::pr-Zm_miR166m_miR_bm3d::t-NOS. As described above, the artificial miRNA precursor pr-Zm_miR166m_miR_bm3d is designed to downregulate the maize COMT transcript. The quantitative analysis of the expression of ZmCOMT in T1 transgenic plants containing pr-Zm_miR166m_miR_bm3d showed a decrease in ZmCOMT transcripts (Table 2). The trait effect expected to result from down-regulation of the COMT transcript was also demonstrated in cell wall digestibility test results (Table 4).

We further tested the ZmCOMT promoter by adding an enhancer by making construct 7, which contains the transgene cassette of p-ZmCOMT:1-Met1-1::pr-Zm_miR166m_miR_bm3d::t-NOS. Expression analysis of ZmCOMT in TO transgenic plants showed a greater decrease in ZmCOMT transcripts (Table 2) in plants with the enhancer, than in TO plants carrying the cassette of construct 1 without the enhancer (data not shown).

The results from analysis of the ZmCOMT promoter with the transgene indicate that it functions strongly enough in cells or tissues where lignification occurs to drive expression of the miRNA precursor and reduce ZmCOMT transcript levels. This result indicates that the failure to detect GUS expression driven by the ZmCOMT promoter (Example 11) is likely due to a very weak expression of the promoter in tissues at the time of sampling.

Altogether, the data from tests with GUS expression and transgene efficacy indicate that the ZmCOMT promoter described above has different functional characteristics from the previously characterized ZmCOMT promoter (Capellades et al., Plant Mol. Biol., 1996, 31:307-322) and that the present promoter is useful for expressing a gene of interest having a unique and novel transcription activity. Due to its specificity, this promoter can be useful in regulating lignin biosynthesis and improving cell wall digestibility while reducing unfavorable agronomic performance.

### Example 13

### Cloning and Construction of a Transgenic Expression Cassette and Super-Binary Vector Comprising the ZmMYB42 Transcription Factor

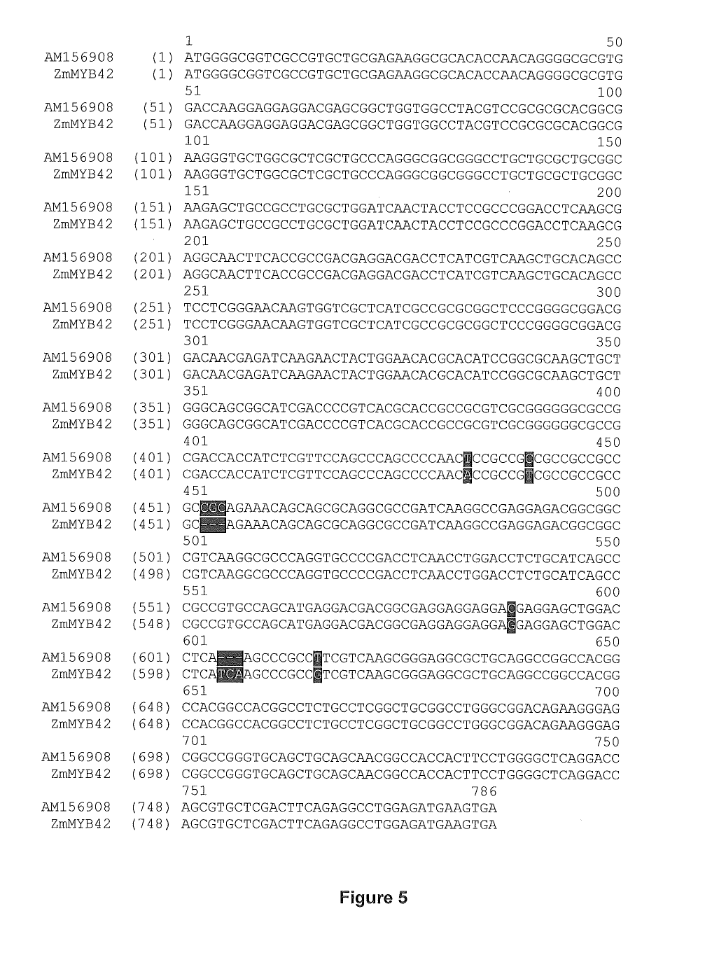

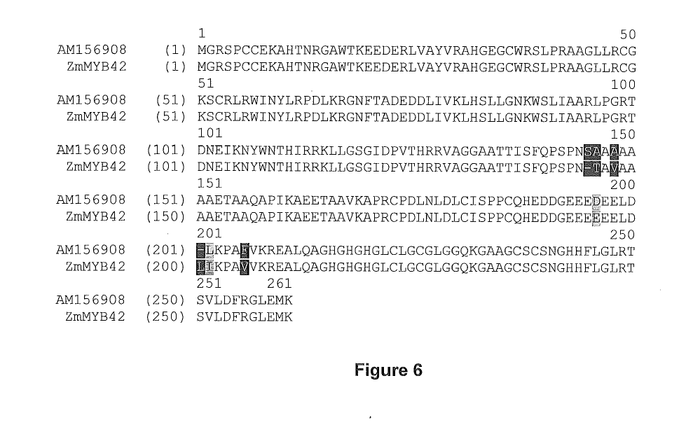

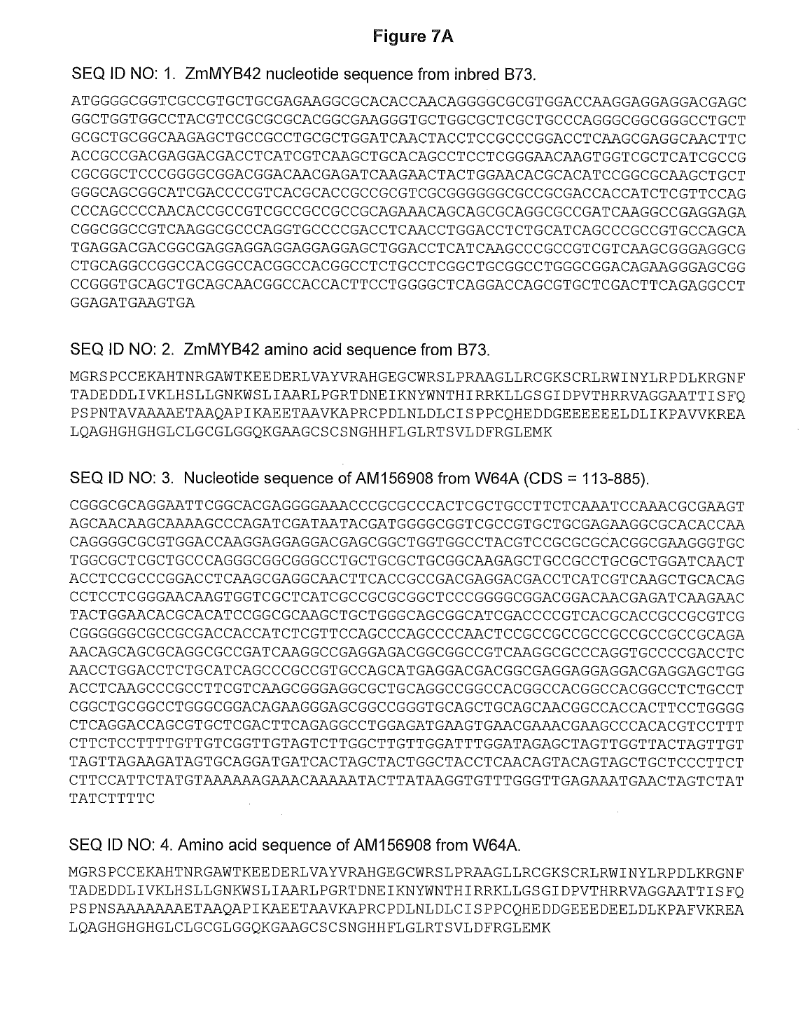

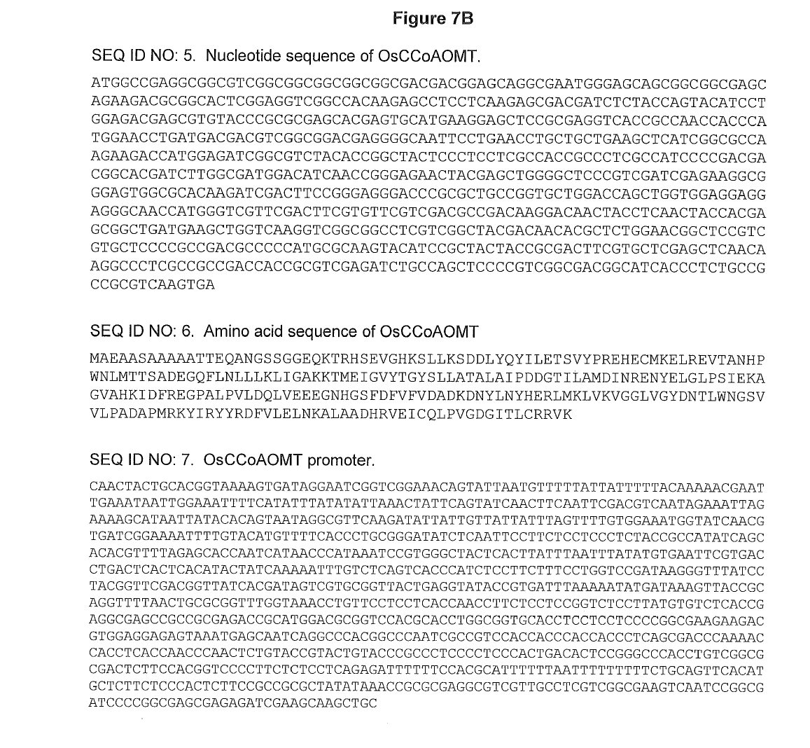

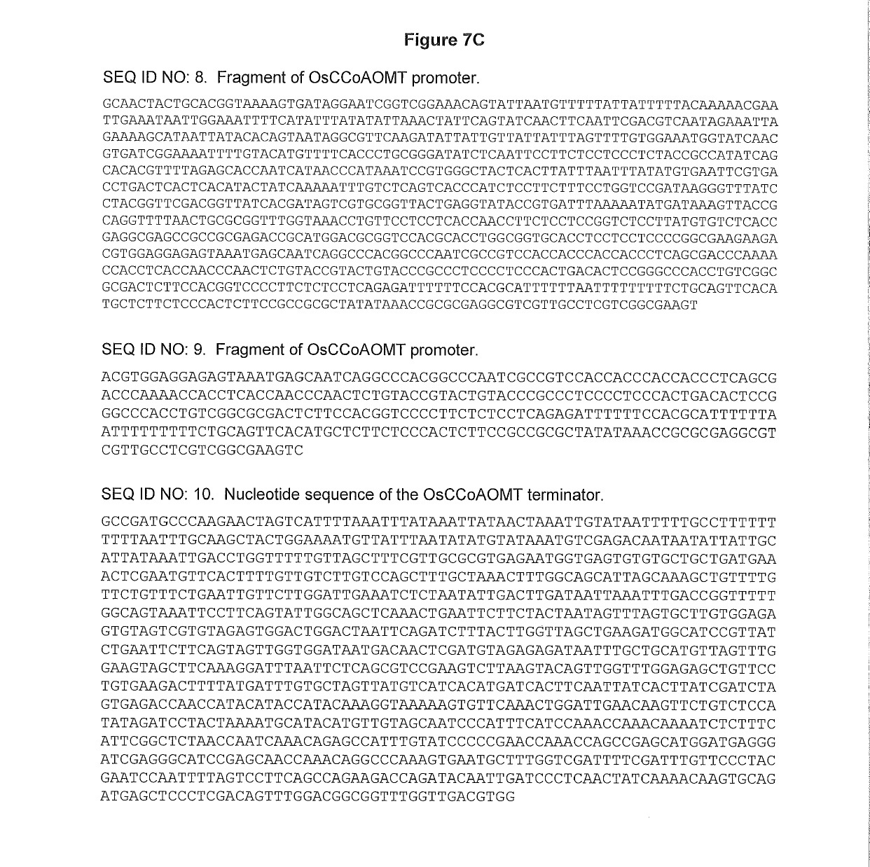

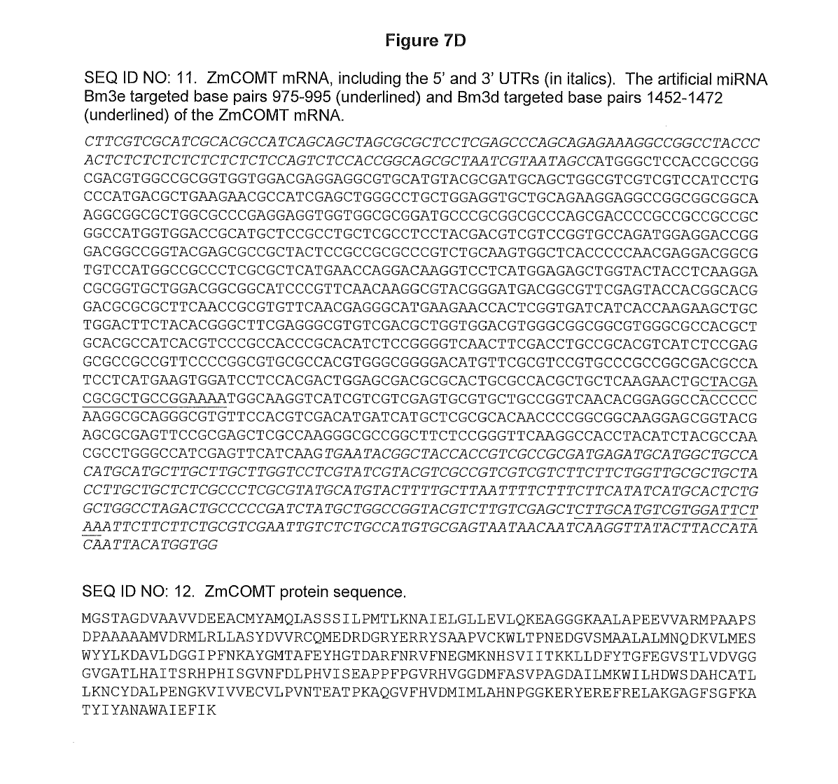

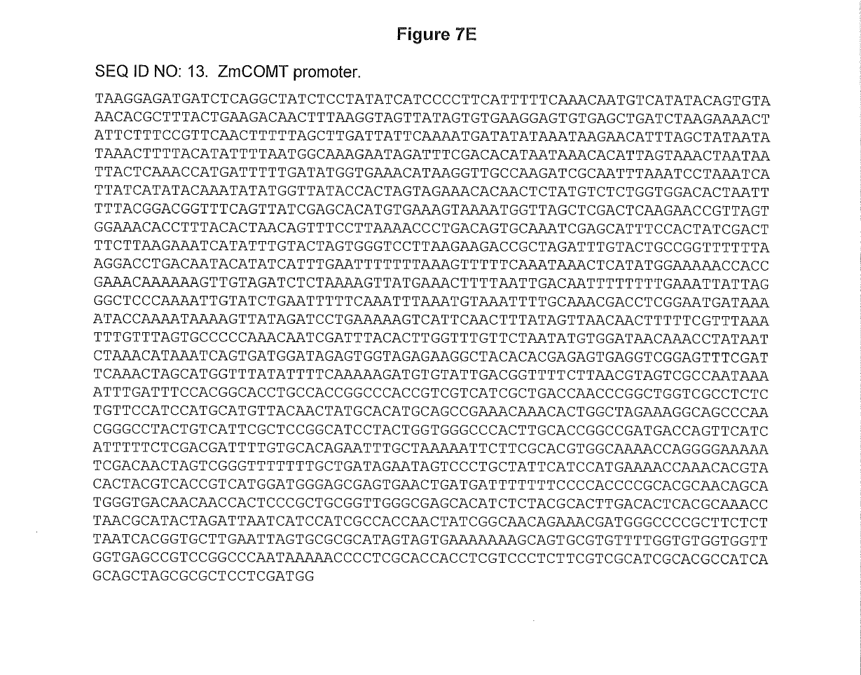

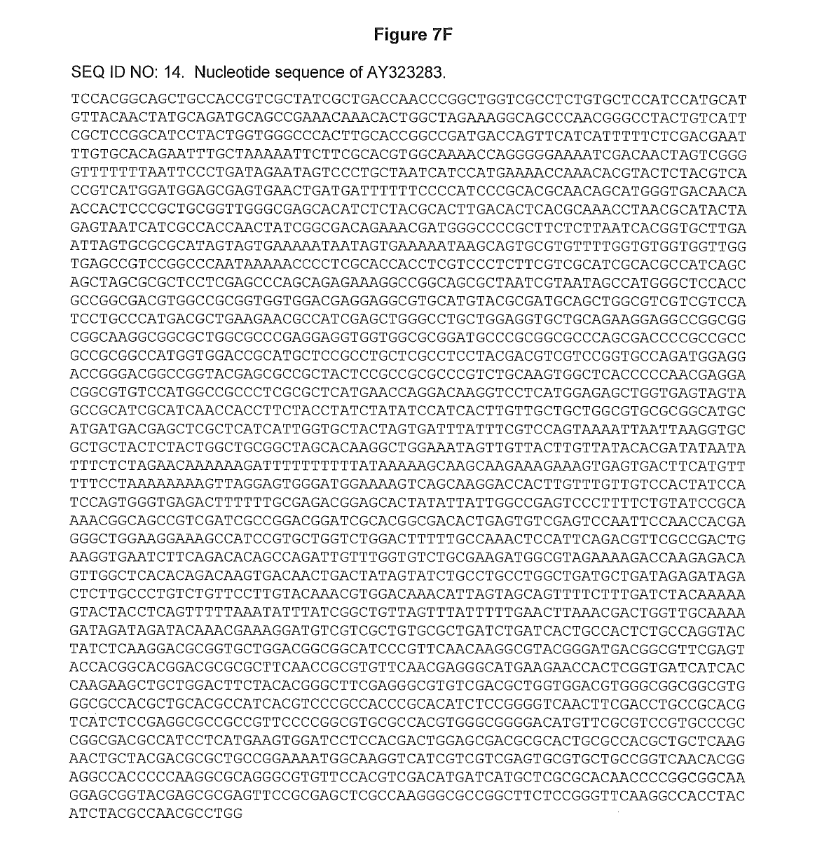

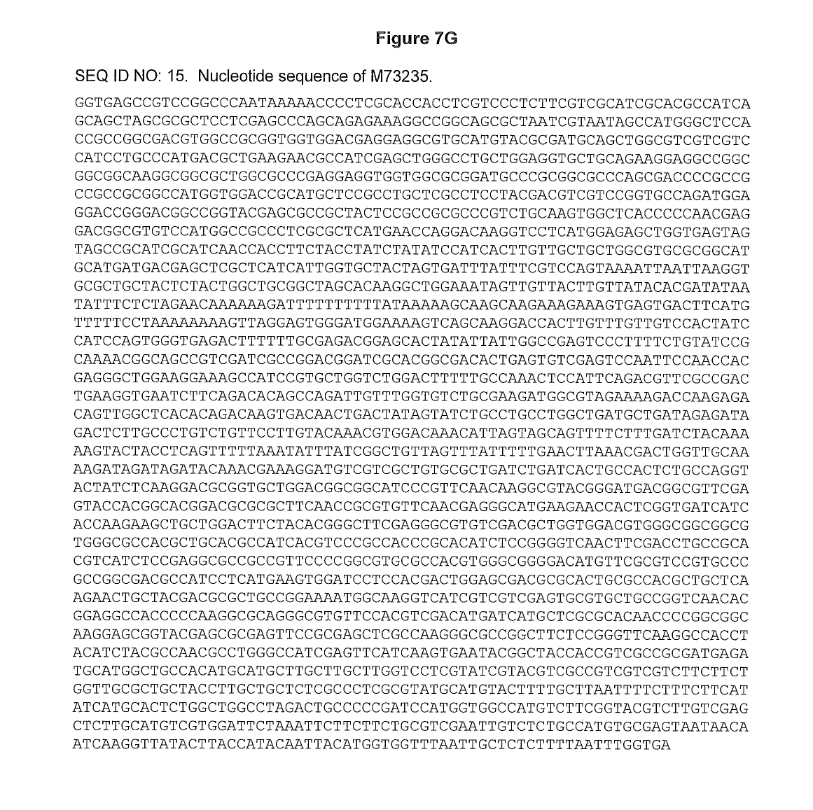

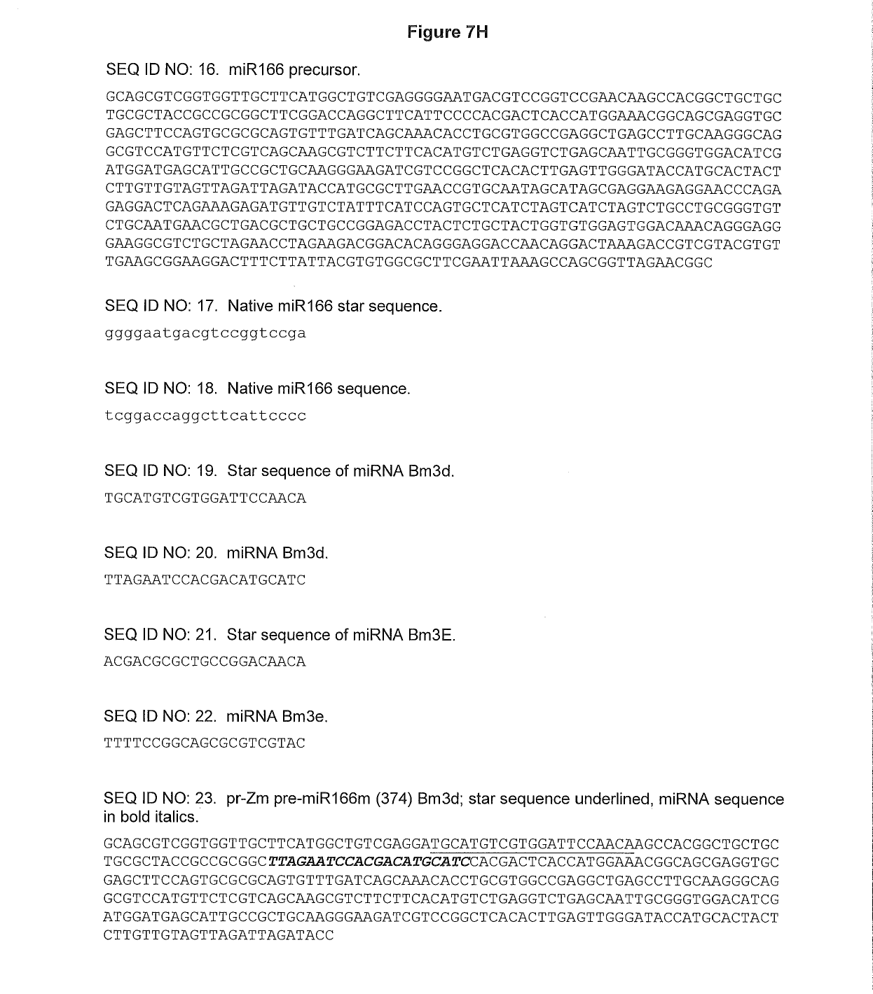

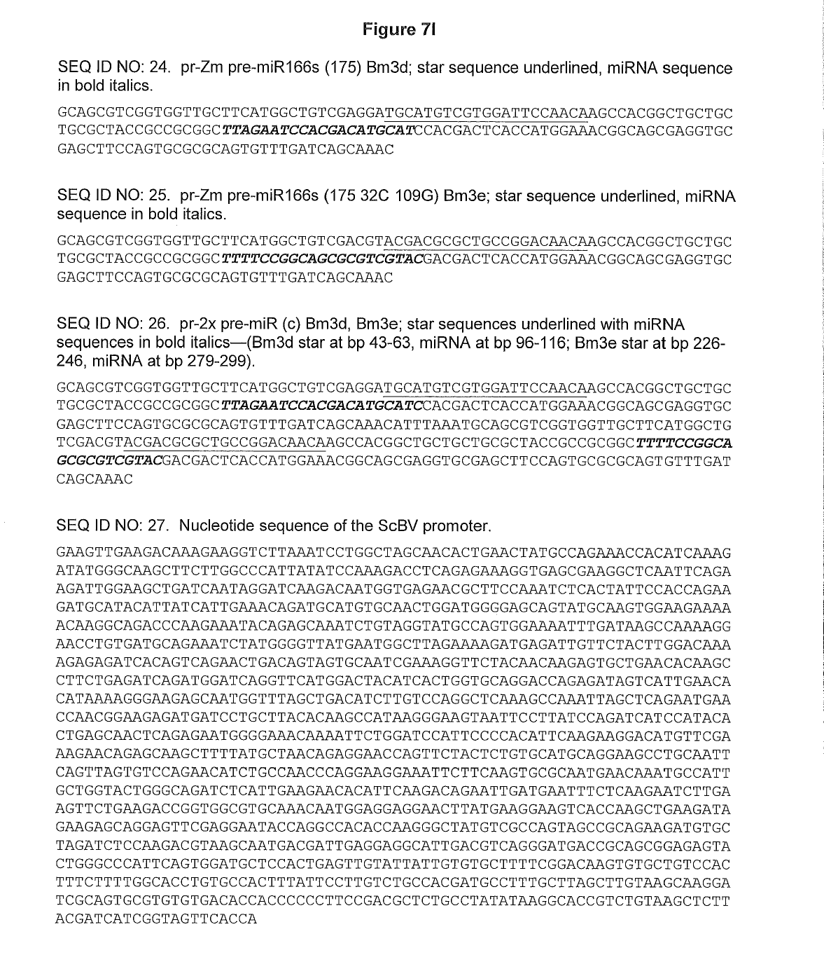

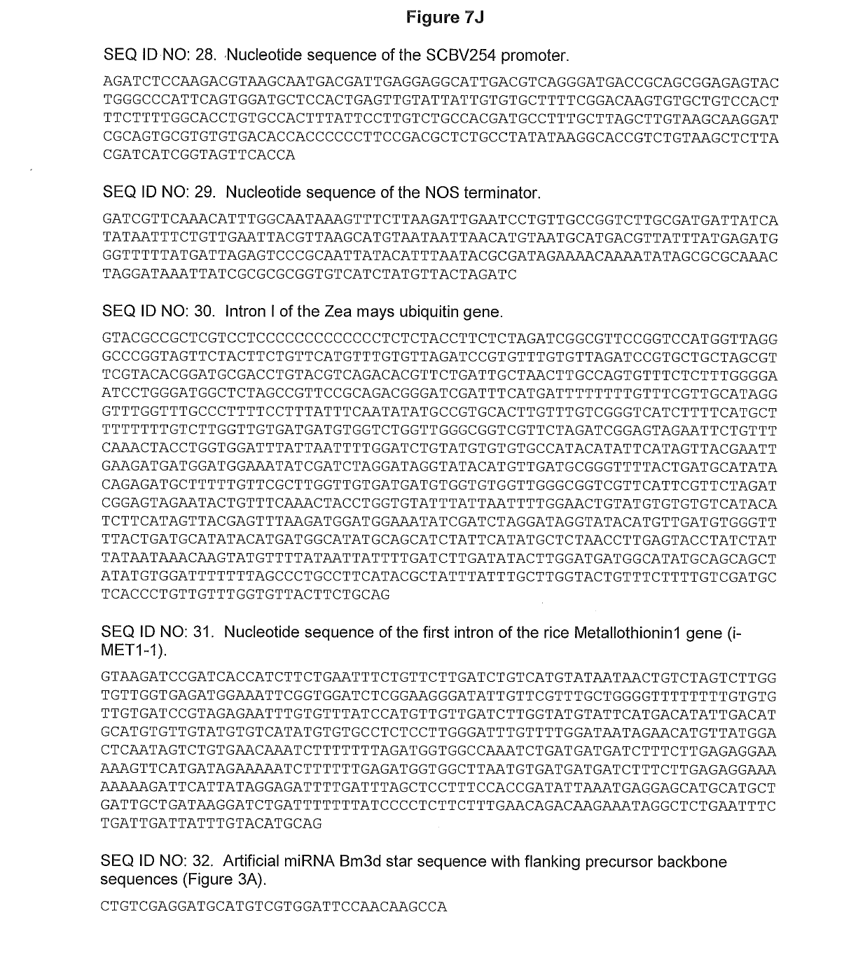

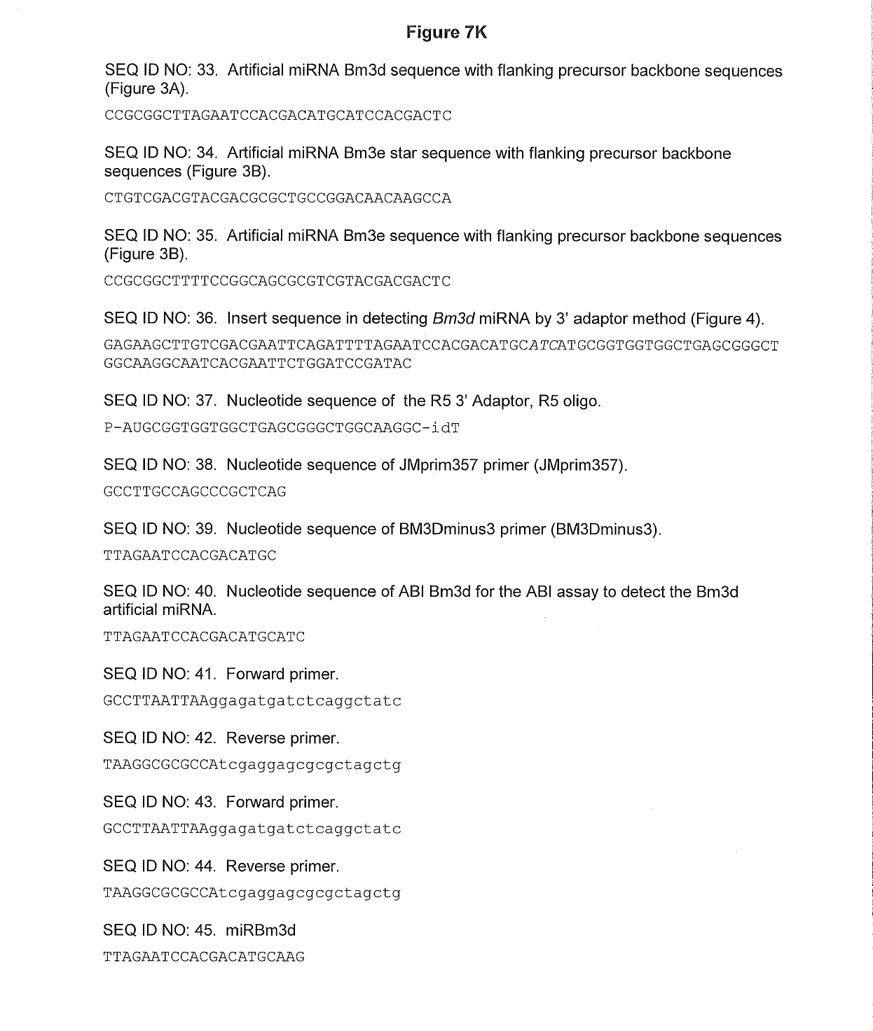

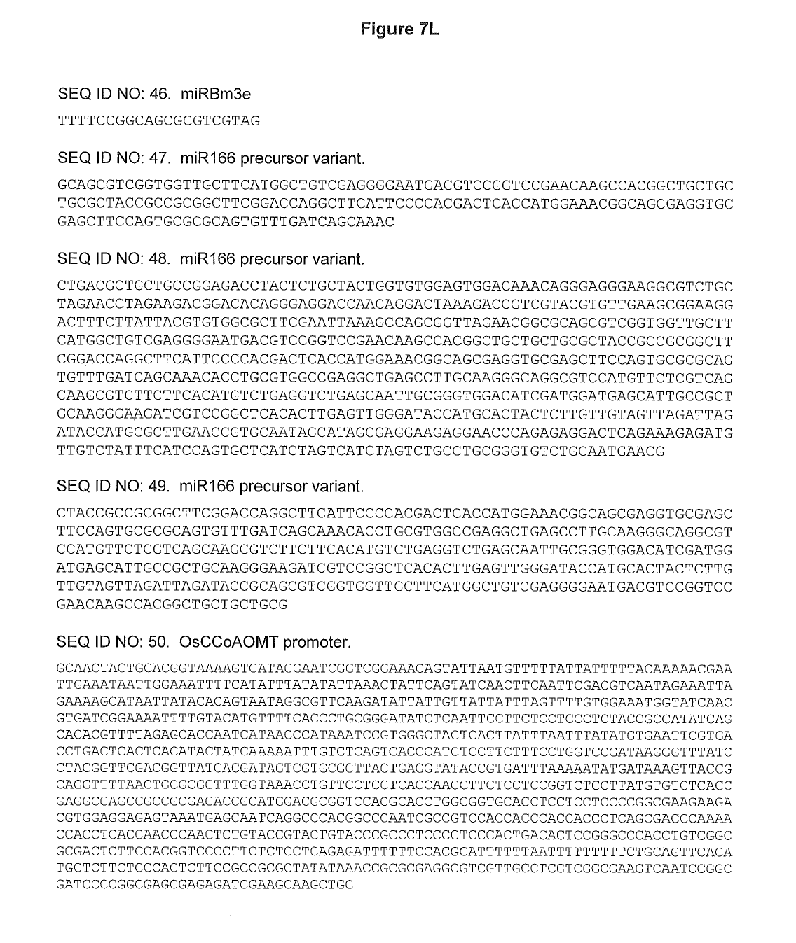

The ZmMYB42 transcription factor sequence was cloned from maize inbred line B73 (SEQ ID NO: 1). Alignment of the polynucleotide (FIG. 5) and protein (FIG. 6) sequences of the cloned B73 ZmMYB42 with AM156908 from W64A (Formale et al., Plant Mol. Biol., 2006, 62:809-823) identified polymorphisms between the two sequences. Expression cassettes comprising the ZmMYB42 transcription factor were constructed and designated construct 12 and construct 13. Construct 12 contains ZmMYB42 under transcriptional control of the rice Caffeoyl CoA-O-methyltransferase (OsCCoAMOT) promoter fused to the first intron of the rice Metallothionin1 gene (iMet1-1). Construct 12 also contains the OsCCoAOMT terminator sequence. Construct 13 contains ZmMYB42 under transcriptional control of p-ZmCOMT and contains the NOS terminator. The constructs were transformed into maize as described in Example 3 above.

TABLE 8
Constructs with ZmMYB42 transcription factor

| Construct | Cassette component | SEQ ID NOs |
|---|---|---|
| 12 | p-OsCCoAOMT::i-Met1-1:: ZmMYB42:: | 7, 31, 1, 10 |
| | t-CoA3′UTRterm | |
| 13 | p-ZmCOMT:: ZmMYB42::t-NOS | 13, 1, 29 |

### Example 14

### COMT Expression Analysis in Transgenic Maize Transformed with Construct 12

Transgenic maize plants transformed with construct 12 were analyzed for ZmCOMT expression as described in Example 4. As shown in Table 9 below, several transgenic lines exhibited reduced ZmCOMT expression.

TABLE 9
ZmCOMT expression measured by qRT-PCR in T0 generation
transgenic plants relative to Wild Type (WT) control.

| | | | COMT Exp. |
|---|---|---|---|
| Construct | Generation | Event Id | (% WT control) |
| 12 | T0 | 12W2 | 44.68 |
| | | 12X2 | 48.31 |
| | | 12Y2 | 54.64 |
| | | 12Z2 | 62.16 |
| | | 12A3 | 62.28 |
| | | 12B3 | 79.16 |
| | | 12C3 | 91.18 |
| | | 12D3 | 96.63 |

### Example 15

### Digestibility Test

The cell wall neutral detergent fibre (NDF) digestibility of transgenic plants transformed with construct 12 was determined as described in Example 6. The test results are summarized in Table 10. Two transgenic lines showed increased NDF digestibility in leaves relative to non-transgenic controls.

TABLE 10
In situ digestibility tests of stem and leaf tissues (unit = % DM).

| | | Stem | | Leaf | |
|---|---|---|---|---|---|
| | | NDF | NDFD | NDF | NDFD |
| Construct | Event | (% DM) | (% DM) | (% DM) | (% DM) |
| 12 | 12X2 | 44.02 | 46.07 | 45.58 | 52.49 |
| | 12B3 | 41.84 | 52.99 | 47.04 | 58.58 |

| | | | | |
|---|---|---|---|---|
| Control (non-transgenic) | NA | NA | 46.20 | 38.23 |

NDF = Neutral detergent fiber, NDFD = 20-h digestibility of NDF, DM = Dry matter.

### Example 16

### In Vitro Digestibility of F1 Hybrid Plants

Two F1 hybrids from crosses of each transgenic event with two testers (nutritionally enhanced and yellow dent) were grown in two field locations with six replications per location in a randomized complete block design. Six whole plants were collected for whole plant analysis. Stem sections from six additional plants were collected for stalk segment analysis. These stem sections consisted of the four internodes above and three internodes below the ear node. No major negative influences on the crop due to weeds, disease or insects were noted. Harvest was conducted at one half to two thirds milk line. In vitro NDFD at 30 hr (NDFD30) was measured by standard NIRS (near-infrared spectroscopy). The test results are summarized in Table 11.

TABLE 11
In vitro digestibility of whole plant and stalk segments by NIRS (Unit = % DM).

| | | Tester 1 (nutritionally enhanced) | | | | Tester 2 (yellow dent) | | | |
|---|---|---|---|---|---|---|---|---|---|
| | | Whole Plant | | Stalk Segment | | Whole Plant | | Stalk Segment | |
| | | NDF | NDFD30 | NDF | NDFD30 | NDF | NDFD30 | NDF | NDFD30 |
| Construct | Description | (% DM) | (% DM) | (% DM) | (% DM) | (% DM) | (% DM) | (% DM) | (% DM) |
| 12 | Control (C) | 47.9 | 28.5 | 45.7 | 24.3 | 47.9 | 28.5 | 45.7 | 24.3 |
| | Event 12X2 (T) | 49 | 29 | 47 | 25.9 | 49 | 29 | 47 | 25.9 |
| | (T/C)% | 106 | 102 | 101 | 102 | 92 | 103 | 98 | 106 |
| | (T/C)% | 102 | 102 | 103 | 106 | 102 | 102 | 103 | 106 |
| | p-value | 0.39 | 0.7 | 0.52 | 0.21 | 0.39 | 0.7 | 0.52 | 0.21 |
| | Event 12B3 (T) | 49.8 | 29 | 48.1 | 25.7 | 49.8 | 29 | 48.1 | 25.7 |
| | T − C | −1.1 | 1.9 | 3.1 | 0.5 | −0.7 | 2.4 | −0.8 | 1.3 |
| | (T/C)% | 104 | 102 | 105 | 105 | 104 | 102 | 105 | 105 |
| | p-value | 0.21 | 0.72 | 0.19 | 0.27 | 0.21 | 0.72 | 0.19 | 0.27 |
| 13 | Control (C) | 40.5 | 48.6 | 60.4 | 32 | 42 | 48.4 | 64.1 | 27.2 |
| | Event 13E3 (T) | 39.7 | 49.6 | 63 | 30 | 41.2 | 50.4 | 67.5 | 28.3 |
| | T − C | −0.9 | 0.9 | 2.6 | −2 | −0.8 | 2 | 3.3 | 1.1 |
| | (T/C)% | 98 | 102 | 104 | 94 | 98 | 104 | 105 | 104 |
| | p-value | 0.7 | 0.72 | 0.18 | 0.5 | 0.66 | 0.61 | 0.17 | 0.59 |
| | Event 13F3 (T) | 40.5 | 50 | 64.5 | 29.5 | 41.7 | 50.2 | 66.3 | 27.3 |
| | T − C | −0.1 | 1.4 | 4.1 | −2.5 | −0.3 | 1.8 | 2.2 | 0.1 |
| | (T/C)% | 100 | 103 | 107 | 92 | 99 | 104 | 103 | 100 |
| | p-value | 0.97 | 0.43 | 0 | 0.42 | 0.89 | 0.6 | 0.44 | 0.95 |

DM = dry matter, NDF = Neutral detergent fiber, NDFD30 = 30-h digestibility of NDF.

### Example 17

### Downregulation Efficiency of Artificial miRNA Targeting COMT Across Diverse Germplasms

Downregulation efficiency of miRNA can greatly depend on the homology of a miRNA sequence to the target sequence. Polymorphisms such as SNPs and small INDELs (insertions and deletions) exist for a gene in different genetic backgrounds or germplasms and influence the level of downregulation of a gene by a miRNA sequence. To test the efficiency of a Bm3d miRNA in downregulation of COMT transcripts across diverse germplasms, event 3P of construct 3 and its non-transgenic isoline control inbred were crossed with 72 different corn inbreds. F1 hybrid plant seedlings were grown in small pots in a greenhouse. Two of the oldest leaves were sampled 21-22 days after planting (at about the V3 or V4 stage). Real time qRT-PCR was run in 3 replicates per hybrid line. The data are shown in Table 12. The expression of COMT was shown as a ratio (COMT/TUA4), which is a relative expression value compared to that of internal reference gene TUA4 (tubulin alpha-4 chain). The data showed that the COMT gene expressed at levels ranging from 5.4% to 24.65% compared to the expression of their respective wild type (WT) control non-transgenic isoline hybrid. This demonstrates that Bm3d miRNA is very effective in downregulating the expression of COMT in diverse germplasm lines.

TABLE 12
Measurement of COMT downregulation by
artificial miRNA in diverse germplasms.

| | | | | | COMT |
|---|---|---|---|---|---|
| | | | | | expres- |
| | Non- | | | | sion |
| | transgenic | | Transgenic | | (% WT |
| Testers | COMT/TUA4 | STDEV | COMT/TUA4 | STDEV | control) |
| Tester 3 | 3.8498 | 0.60250 | 0.4532 | 0.07145 | 11.77 |
| Tester 4 | 2.7923 | 0.12191 | 0.2289 | 0.03585 | 8.20 |
| Tester 5 | 3.1873 | 0.74860 | 0.3027 | 0.02826 | 9.50 |
| Tester 6 | 1.9100 | 0.06900 | 0.3664 | 0.02090 | 19.18 |
| Tester 7 | 2.9040 | 0.33744 | 0.2430 | 0.04721 | 8.37 |
| Tester 8 | 1.6784 | 0.54108 | 0.3135 | 0.09406 | 18.68 |
| Tester 9 | 2.0263 | 0.12550 | 0.2512 | 0.17462 | 12.40 |
| Tester 10 | 6.7972 | 1.41955 | 0.3672 | 0.04460 | 5.40 |
| Tester 11 | 4.2507 | 1.28162 | 0.4910 | 0.11593 | 11.55 |
| Tester 12 | 3.2695 | 0.26276 | 0.4006 | 0.09536 | 12.25 |
| Tester 13 | 3.2058 | 0.30522 | 0.5898 | 0.21169 | 18.40 |
| Tester 14 | 3.1262 | 0.81288 | 0.3320 | 0.07226 | 10.62 |
| Tester 15 | 2.4790 | 0.40193 | 0.2496 | 0.03165 | 10.07 |
| Tester 16 | 1.9670 | 0.18389 | 0.3183 | 0.04593 | 16.18 |
| Tester 17 | 2.4818 | 0.70248 | 0.3340 | 0.12835 | 13.46 |
| Tester 18 | 2.1685 | 0.28252 | 0.2660 | 0.13097 | 12.27 |
| Tester 19 | 2.4746 | 0.77410 | 0.1557 | 0.00018 | 6.29 |
| Tester 20 | 2.0624 | 0.48747 | 0.4229 | 0.15340 | 20.50 |
| Tester 21 | 2.6287 | 0.28939 | 0.1874 | 0.05846 | 7.13 |
| Tester 22 | 1.3885 | 0.15820 | 0.1695 | 0.00000 | 12.21 |
| Tester 23 | 3.6823 | 0.99312 | 0.4241 | 0.18115 | 11.52 |
| Tester 24 | 4.3000 | 0.19000 | 0.4669 | 0.12474 | 10.83 |
| Tester 25 | 0.9664 | 0.71738 | 0.2045 | 0.01801 | 21.16 |
| Tester 26 | 1.5871 | 0.08259 | 0.3913 | 0.09526 | 24.65 |
| Tester 27 | 3.5149 | 0.86533 | 0.3085 | 0.09154 | 8.78 |
| Tester 28 | 1.8302 | 0.08508 | 0.2810 | 0.03151 | 15.35 |
| Tester 29 | 1.9048 | 0.18633 | 0.1721 | 0.01839 | 9.03 |
| Tester 30 | 1.9045 | 0.24255 | 0.3324 | 0.06658 | 17.45 |
| Tester 31 | 1.4897 | 0.13777 | 0.3075 | 0.02190 | 20.64 |
| Tester 32 | 1.9826 | 0.20568 | 0.1415 | 0.02002 | 7.14 |
| Tester 33 | 1.7397 | 0.14103 | 0.2285 | 0.01312 | 13.14 |
| Tester 34 | 1.6580 | 0.20353 | 0.2818 | 0.02239 | 17.00 |
| Tester 35 | 1.0496 | 0.37097 | 0.1628 | 0.03884 | 15.51 |
| Tester 36 | 2.6042 | 0.37600 | 0.3646 | 0.03229 | 14.00 |
| Tester 37 | 1.5491 | 0.17795 | 0.1901 | 0.03591 | 12.27 |
| Tester 38 | 2.7110 | 0.25456 | 0.3400 | 0.02666 | 12.54 |
| Tester 39 | 1.2556 | 0.13521 | 0.2270 | 0.01358 | 18.08 |
| Tester 40 | 1.2788 | 0.19512 | 0.1741 | 0.03007 | 13.61 |
| Tester 41 | 1.6627 | 0.11362 | 0.2164 | 0.01907 | 13.02 |
| Tester 42 | 2.2072 | 0.33591 | 0.3299 | 0.02288 | 14.95 |
| Tester 43 | 3.1068 | 0.47777 | 0.2739 | 0.01783 | 8.82 |
| Tester 44 | 2.3508 | 0.45874 | 0.3007 | 0.02073 | 12.79 |
| Tester 45 | 3.0500 | 0.46380 | 0.2798 | 0.03815 | 9.17 |
| Tester 46 | 2.8400 | 0.29000 | 0.2105 | 0.05864 | 7.41 |
| Tester 47 | 1.7259 | 0.38462 | 0.1512 | 0.01433 | 8.76 |
| Tester 48 | 3.1760 | 0.59226 | 0.3036 | 0.02672 | 9.56 |
| Tester 49 | 2.2953 | 0.60957 | 0.2200 | 0.04700 | 9.58 |
| Tester 50 | 2.1909 | 0.30536 | 0.2543 | 0.03944 | 11.61 |
| Tester 51 | 2.4037 | 0.58074 | 0.4688 | 0.05507 | 19.50 |
| Tester 52 | 1.2212 | 0.27481 | 0.1893 | 0.01960 | 15.50 |
| Tester 53 | 2.6556 | 0.57957 | 0.3654 | 0.00585 | 13.76 |
| Tester 54 | 1.4531 | 0.27353 | 0.2412 | 0.06121 | 16.60 |
| Tester 55 | 1.3624 | 0.29269 | 0.1501 | 0.03182 | 11.02 |
| Tester 56 | 2.8544 | 0.21494 | 0.2773 | 0.02998 | 9.72 |
| Tester 57 | 1.1811 | 0.71992 | 0.2270 | 0.02930 | 19.22 |
| Tester 58 | 4.6386 | 0.19486 | 0.6167 | 0.08122 | 13.30 |
| Tester 59 | 1.7705 | 0.00000 | 0.2527 | 0.03591 | 14.27 |
| Tester 60 | 1.1712 | 0.31427 | 0.1652 | 0.02395 | 14.11 |
| Tester 61 | 1.5443 | 0.20048 | 0.2193 | 0.00954 | 14.20 |
| Tester 62 | 1.8719 | 0.09263 | 0.2311 | 0.02063 | 12.35 |
| Tester 63 | 2.0597 | 0.52383 | 0.3454 | 0.01887 | 16.77 |
| Tester 64 | 1.6344 | 0.16424 | 0.1902 | 0.01820 | 11.64 |
| Tester 65 | 1.4563 | 0.32496 | 0.1767 | 0.02774 | 12.13 |
| Tester 66 | 1.9133 | 0.44405 | 0.2000 | 0.03232 | 10.45 |
| Tester 67 | 2.4238 | 0.16630 | 0.2544 | 0.08552 | 10.49 |
| Tester 68 | 2.3723 | 0.13700 | 0.1837 | 0.05213 | 7.74 |
| Tester 69 | 1.9140 | 0.12611 | 0.2364 | 0.04725 | 12.35 |
| Tester 70 | 1.5457 | 0.08835 | 0.1796 | 0.02355 | 11.62 |
| Tester 71 | 1.0011 | 0.11956 | 0.1136 | 0.02624 | 11.35 |
| Tester 72 | 1.4565 | 0.38426 | 0.3186 | 0.04280 | 21.87 |
| Tester 73 | 1.2402 | 0.09488 | 0.1726 | 0.00784 | 13.92 |
| Tester 74 | 1.8931 | 0.00000 | 0.2183 | 0.00618 | 11.53 |

1. An expression cassette for down-regulating lignin biosynthesis in a plant comprising:
(a) a transcription regulating nucleotide sequence of a Caffeoyl-CoA O-methyltransferase (CCoAOMT) gene, and
(b) a nucleic acid sequence encoding the maize transcription factor MYB42 (ZmMYB42) operably linked to said transcription regulating nucleotide sequence.

2. The expression cassette of claim 1, wherein the transcription regulating nucleotide sequence is from a rice Caffeoyl-CoA O-methyltransferase (OsCCoAOMT) gene.

3. The expression cassette of claim 1, wherein the transcription regulating nucleotide sequence comprises the polynucleotide sequence of SEQ ID NO: 7.

4. The expression cassette of claim 1, wherein the gene encoding the maize transcription factor MYB42 (ZmMYB42) comprises a polynucleotide sequence selected from the group consisting of:
(a) the polynucleotide sequence of SEQ ID NO: 1,
(b) a polynucleotide sequence encoding a polypeptide having the amino acid sequence of SEQ ID NO: 2, and
(c) a polynucleotide sequence encoding a polypeptide having at least 95% identity to SEQ ID NO: 2 and having activity of a MYB transcription factor.

5. An expression cassette comprising:
(a) a transcription regulating nucleotide sequence, and operably linked thereto
(b) a miR166 precursor sequence that is engineered to produce a miRNA sequence which reduces expression of a gene in the lignin biosynthesis pathway.

6. The expression cassette of claim 5, wherein, prior to engineering, the miR166 precursor sequence comprises a polynucleotide sequence selected from the group consisting of:
(a) the polynucleotide sequence of SEQ ID NO: 16;
(b) a polynucleotide sequence having at least 70% sequence identity with the sequence of SEQ ID NO: 16 that is capable of producing a miRNA sequence; and
(c) a functional fragment of the polynucleotide sequence of SEQ ID NO: 16 that is capable of producing a miRNA sequence.

7. The expression cassette of claim 5, wherein the miR166 precursor sequence is engineered by replacing a first segment of about 19-24 contiguous nucleotides located between positions corresponding to about nucleotide 32 and nucleotide 55 of SEQ ID NO: 16 with a first nucleotide sequence of about 19-24 nucleotides, and a second segment of about 19-24 contiguous nucleotides located between positions corresponding to about nucleotide 86 and nucleotide 109 of SEQ ID NO: 16 with a second nucleotide sequence of about 19-24 nucleotides, wherein the first nucleotide sequence and the second nucleotide sequence are substantially complementary to each other, and the second nucleotide sequence is substantially complementary to a portion of a mRNA transcribed from the gene in the lignin biosynthesis pathway.

8. The expression cassette of claim 7, wherein the first segment comprises the polynucleotide sequence of SEQ ID NO: 17 and the second segment comprises the polynucleotide sequence of SEQ ID NO: 18.

9. The expression cassette of claim 7, wherein the second nucleotide sequence is substantially complementary to 5′-UTR, 3′-UTR, or coding region of a mRNA transcribed from the gene in the lignin biosynthesis pathway.

10. The expression cassette of claim 5, wherein the gene in the lignin biosynthesis pathway encodes a polypeptide selected from the group consisting of phenylalanine ammonia lyase (PAL), cinnamate 4-hydrolase (C4H), 4-coumarate:CoA ligase (4CL), hydroxycinnamoyl-CoA:shikimate and quinate hydroxyl-cinnamoyl transferase (HCT), p-coumarate 3-hydroxylase (C3H), caffeoyl-CoA 3-O-methyltransferase (CCoAOMT), cinnamoyl-CoA reductase (CCR), cinnamyl alcohol dehydrogenase (CAD), sinapyl alcohol dehydrogenase (SAD), ferulate 5-hydroxylase (F5H), caffeic acid O-methyltransferase (COMT), hydroxycinnamaldehyde dehydrogenase (HCALDH), peroxidase, and laccase.

11. The expression cassette of claim 10, wherein the gene in the lignin biosynthesis pathway is a caffeic acid O-methyltransferase (COMT) gene.

12. The expression cassette of claim 11, wherein the gene in the lignin biosynthesis pathway is a maize caffeic acid O-methyltransferase (ZmCOMT) gene.

13. The expression cassette of claim 5, wherein the transcription regulating nucleotide sequence is a constitutive promoter, a tissue-specific or tissue-preferential promoter, an inducible promoter, or a developmentally regulated promoter.

14. The expression cassette of claim 5, wherein the transcription regulating nucleotide sequence is from a caffeic acid O-methyltransferase (COMT) gene.

15. The expression cassette of claim 5, wherein the transcription regulating nucleotide sequence is from a maize caffeic acid O-methyltransferase (ZmCOMT) gene.

16. The expression cassette of claim 5, wherein the transcription regulating nucleotide sequence comprises the polynucleotide sequence of SEQ ID NO: 13.

17. The expression cassette of claim 7, wherein the first nucleotide sequence that replaces the first segment comprises
(a) the polynucleotide sequence of SEQ ID NO: 19 or SEQ ID NO: 21, or
(b) a polynucleotide sequence having at least 70% sequence identity to the polynucleotide sequence of SEQ ID NO: 19 or SEQ ID NO: 21,
and the second nucleotide sequence that replaces the second segment comprises
(i) the polynucleotide sequence of SEQ ID NO: 20 or SEQ ID NO: 22, or
(ii) a polynucleotide sequence having at least 70% sequence identity to the polynucleotide sequence of SEQ ID NO: 20 or SEQ ID NO: 22,
wherein the first nucleotide sequence and the second nucleotide sequence remain substantially complementary to each other, and the second nucleotide sequence is capable of reducing expression of a maize COMT gene.

18. The expression cassette of claim 5, wherein the miR166 precursor sequence is engineered to comprise the polynucleotide sequence of SEQ ID NO: 23, SEQ ID NO: 24, or SEQ ID NO: 25.

19. The expression cassette of claim 5, further comprises a second miR166 precursor sequence.

20. The expression cassette of claim 19, wherein the second miR166 precursor sequence is engineered to produce a second miRNA sequence which reduces expression of a gene in the lignin biosynthesis pathway.

21. The expression cassette of claim 19, comprising the polynucleotide sequence of SEQ ID NO: 26.

22. A transcription regulating nucleotide sequence comprises:
(a) the polynucleotide sequence of SEQ ID NO: 13,
(b) a polynucleotide sequence comprising at least 750 nucleotides and having at least 90% identity to the nucleotide sequence of SEQ ID NO: 13, wherein said nucleotide sequence is capable of driving expression of a heterologous nucleic acid sequence that is operably linked thereto, or
(c) a fragment of SEQ ID NO: 13, wherein the fragment comprises at least 750 nucleotides and is capable of driving expression of a heterologous nucleic acid sequence that is operably linked thereto.

23. An expression cassette comprising:
(a) the transcription regulating nucleotide sequence of claim 22, and
(b) a nucleic acid sequence operably linked to and heterologous in relation to said transcription regulating nucleotide sequence.

24. The expression cassette of claim 23, wherein expression of the nucleic acid sequence results in expression of a protein, or expression of an antisense RNA, a sense RNA, a double-stranded RNA, or a miRNA.

25. The expression cassette of claim 23, wherein expression of the nucleic acid sequence confers to the plant an agronomically valuable trait.

26. The expression cassette of claim 25, wherein the agronomically valuable trait is reduced lignin production, increased crude protein, and/or increased cell wall digestibility.

27. The expression cassette of claim 23, wherein the nucleic acid comprises a nucleotide sequence encoding the maize transcription factor MYB42 (ZmMYB42).

28. The expression cassette of claim 27, wherein the nucleotide sequence comprises a polynucleotide sequence selected from the group consisting of:
(a) the polynucleotide sequence of SEQ ID NO: 1,
(b) a polynucleotide sequence encoding a polypeptide having the amino acid sequence of SEQ ID NO: 2, and
(c) a polynucleotide sequence encoding a polypeptide having at least 95% identity to SEQ ID NO: 2 and having activity of a MYB transcription factor.

29. The expression cassette of claim 1, further comprising a terminator, an enhancer, or a terminator and an enhancer.

30. The expression cassette of claim 29, wherein the terminator is from a Caffeoyl-CoA O-methyltransferase (CCoAOMT) gene.

31. The expression cassette of claim 29, wherein the terminator is from a rice Caffeoyl-CoA O-methyltransferase (OsCCoAOMT) gene.

32. The expression cassette of claim 29, wherein the terminator comprises the polynucleotide sequence of SEQ ID NO: 10.

33. The expression cassette of claim 29, wherein the enhancer is an intron.

34. The expression cassette of claim 33, wherein the intron comprises the polynucleotide sequence of SEQ ID NO: 31.

35. A recombinant construct comprising the expression cassette of claim 1.

36. The recombinant construct of claim 35, comprising at least one further expression cassette.

37. A vector comprising the recombinant construct of claim 35.

38. A plant cell, plant or part thereof, or microorganism comprising the expression cassette of claim 1, a recombinant construct comprising at least one said expression cassette, or a vector comprising said expression cassette or said recombinant construct.

39. A method for producing a transgenic plant or plant cell, comprising
(a) transforming a plant or plant cell with the expression cassette of claim 1, a recombinant construct comprising at least one said expression cassette, or a vector comprising said expression cassette or said recombinant construct, and
(b) optionally regenerating from the plant cell a transgenic plant.

40. A method for increasing digestibility and/or crude protein of a plant, comprising:
(a) transforming a plant or a plant cell with the expression cassette of claim 1, a recombinant construct comprising at least one said expression cassette, or a vector comprising said expression cassette or said recombinant construct,
(b) growing said transformed plant or plant cell,
(c) optionally, regenerating from the plant cell a transgenic plant,
wherein expression of the nucleic acid sequence and/or the miR166 precursor sequence results in down-regulation of lignin biosynthesis in the plant or plant cell and confers an increase in digestibility and/or crude protein of the transgenic plant or part thereof as compared to a corresponding wild-type plant.

41. The method of claim 40, wherein the plant is a monocotyledonous plant or the plant cell or plant part is from a monocotyledonous plant.

42. The method of claim 40, wherein the plant is a maize plant or the plant cell or plant part is from a maize plant.

43. The expression cassette of claim 5, further comprising a terminator, an enhancer, or a terminator and an enhancer.

44. A recombinant construct comprising the expression cassette of claim 5.

45. The recombinant construct of claim 44, comprising at least one further expression cassette.

46. A vector comprising the recombinant construct of claim 44.

47. A plant cell, plant or part thereof, or microorganism comprising the expression cassette of claim 5, a recombinant construct comprising at least one said expression cassette, or a vector comprising said expression cassette or said recombinant construct.

48. A method for producing a transgenic plant or plant cell, comprising
(a) transforming a plant or plant cell with the expression cassette of claim 5, a recombinant construct comprising at least one said expression cassette, or a vector comprising said expression cassette or said recombinant construct, and
(b) optionally regenerating from the plant cell a transgenic plant.

49. A method for increasing digestibility and/or crude protein of a plant, comprising:
(a) transforming a plant or a plant cell with the expression cassette of claim 5, a recombinant construct comprising at least one said expression cassette, or a vector comprising said expression cassette or said recombinant construct,
(b) growing said transformed plant or plant cell,
(c) optionally, regenerating from the plant cell a transgenic plant,
wherein expression of the nucleic acid sequence and/or the miR166 precursor sequence results in down-regulation of lignin biosynthesis in the plant or plant cell and confers an increase in digestibility and/or crude protein of the transgenic plant or part thereof as compared to a corresponding wild-type plant.
